(19) United States
(12) Patent Application Publication
(10) Pub. No.: US 2023/0322691 A1
(43) Pub. Date: Oct. 12, 2023

(54) ORGANIC COMPOUND AND ORGANIC LIGHT-EMITTING ELEMENT

(71) Applicant: CANON KABUSHIKI KAISHA, Tokyo (JP)

(72) Inventors: YOSUKE NISHIDE, Kanagawa (JP); JUN KAMATANI, Tokyo (JP); NAOKI YAMADA, Tokyo (JP); HIROKI OHRUI, Tokyo (JP); HIRONOBU IWAWAKI, Kanagawa (JP); HIROKAZU MIYASHITA, Miyagi (JP)

(21) Appl. No.: 18/297,243

(22) Filed: Apr. 7, 2023

(30) Foreign Application Priority Data
Apr. 12, 2022 (JP) 2022-065793

(51) Int. Cl. C07D 263/57; H10K 50/11; H10K 85/30; H10K 85/60; C07D 413/10; C07D 491/048; C07D 519/00; C07D 293/12; C07D 498/04; C07D 517/04; C07D 491/052; C07D 495/04; C07D 497/04; C07D 513/04; C07D 417/10

(52) U.S. Cl. CPC C07D 263/57; H10K 2101/10; H10K 85/342; H10K 85/622; H10K 85/657; C07D 413/10; C07D 491/048; H10K 85/6574; C07D 519/00; C07D 293/12; C07D 498/04; C07D 517/04; C07D 491/052; C07D 495/04; C07D 497/04; C07D 513/04; C07D 417/10; H10K 50/11

(57) ABSTRACT

An organic compound represented by [1], satisfying (i) and (ii), and represented by [2-1] or [2-2].

A-(B)ₘ [1]
(i) T₁ of the unit A<T₁ of the unit B, and
(ii) HOMO-LUMO orbitals responsible for T₁ transition are present in the unit A,

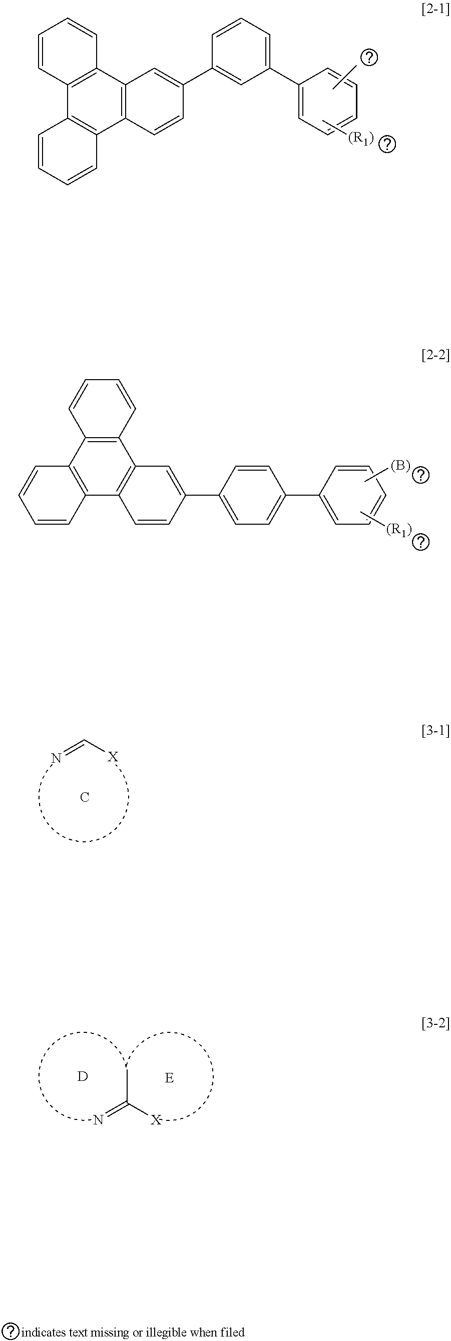

wherein R₁ is selected from alkyl groups and the like, m denotes an integer of 1 or more and 5 or less, and n denotes an integer of 0 or more and 4 or less, the unit B is a fused ring with a partial structure represented by the general formula [3-1] or [3-2] or the like and is bonded to the unit A via a carbon atom, wherein X is selected from an oxygen atom, a sulfur atom, and the like, and the rings C to E independently denote a single ring and may have a deuterium atom or an alkyl group.

### BACKGROUND OF THE INVENTION

### Field of the Invention

The present disclosure relates to an organic compound and an organic light-emitting element including the organic compound.

### Description of the Related Art

An organic light-emitting element (hereinafter sometimes referred to as an “organic electroluminescent element” or an “organic EL element”) is an electronic element that includes a pair of electrodes and an organic compound layer between the electrodes. Electrons and holes are injected from the pair of electrodes to generate an exciton of a light-emitting organic compound in the organic compound layer. When the exciton returns to its ground state, the organic light-emitting element emits light. With recent significant advances in organic light-emitting elements, it is possible to realize low drive voltage, various emission wavelengths, high-speed responsivity, and thin and light light-emitting devices.

Compounds suitable for organic light-emitting elements have been actively developed. This is because a compound that provides an element with good lifetime characteristics is important for high-performance organic light-emitting elements. As compounds developed so far, an organic compound 1-a containing triphenylene and a heterocycle is described in U.S. Patent Application Publication No. 2015/0318487 (PTL 1), and an organic compound 2-a is described in International Publication No. WO 2019/24526 (PTL 2).

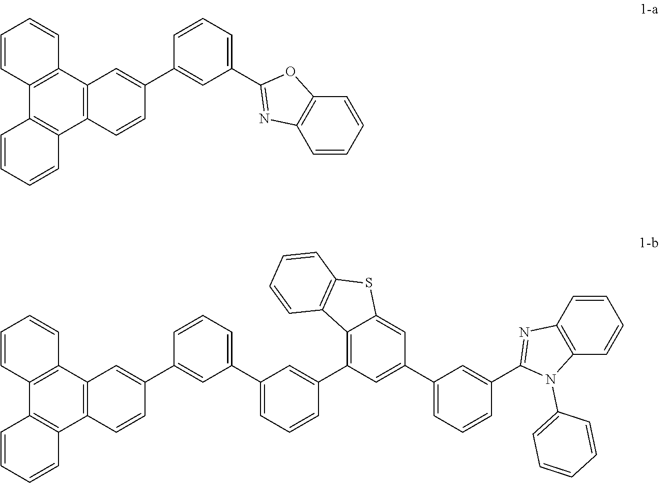 [SMILES: c1cc(-c2ccc3c4ccccc4c4ccccc4c3c2)cc(-c2nc3ccccc3o2)c1 ; c1ccc(-n2c(-c3cccc(-c4cc(-c5cccc(-c6cccc(-c7ccc8c9ccccc9c9ccccc9c8c7)c6)c5)c5c(c4)sc4ccccc45)c3)nc3ccccc32)cc1]

Although PTL 1 and PTL 2 disclose examples of green-light-emitting elements containing the compounds 1-a and 2-a, further improvement of light emission efficiency and durability is desired.

### SUMMARY OF THE INVENTION

In view of the above disadvantages, an embodiment of the present disclosure provides an organic compound and an organic light-emitting element with high light emission efficiency and durability.

An organic compound according to an embodiment of the present disclosure is an organic compound represented by the following formula [1]:

A-(B)ₘ [1]
wherein the organic compound satisfies (i) and (ii),
(i) T₁ of the unit A<T₁ of the unit B, and
(ii) highest occupied molecular orbital (HOMO)-lowest unoccupied molecular orbital (LUMO) orbitals responsible for T₁ transition are present in the unit A, and
the organic compound is represented by the following formula [2-1] or [2-2]:

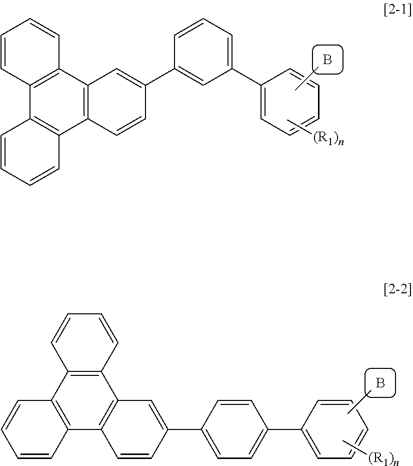

wherein R₁ is independently selected from a deuterium atom, substituted or unsubstituted alkyl groups, substituted or unsubstituted aryl groups, and substituted or unsubstituted heterocyclic groups, provided that R₁ is bonded via a carbon atom when R₁ denotes a substituted or unsubstituted heterocyclic group,
m denotes an integer of 1 or more and 5 or less, and n denotes an integer of 0 or more and 4 or less, and
the unit B is a fused ring with a partial structure represented by the following formula [3-1] or [3-2] or is a combination of the fused ring and a substituted or unsubstituted aromatic ring, and is bonded to the unit A via a carbon atom,

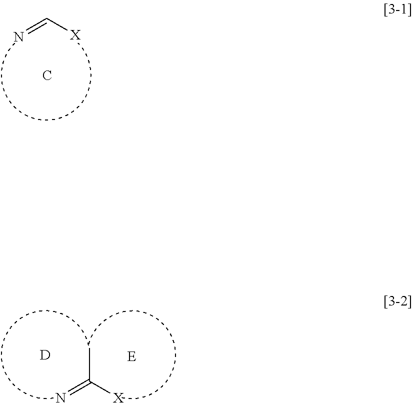

wherein X is selected from an oxygen atom, a sulfur atom, a selenium atom, and a tellurium atom, and
rings C to E independently denote a single ring and may have a deuterium atom or a substituted or unsubstituted alkyl group.

Further features of the present disclosure will become apparent from the following description of exemplary embodiments with reference to the attached drawings.

### BRIEF DESCRIPTION OF THE DRAWINGS

FIG. 1 is a table showing HOMO-LUMO distributions of exemplary compounds and a comparative compound 1-a.

FIG. 2A is a schematic cross-sectional view of an example of a pixel of a display apparatus according to an embodiment of the present disclosure.

FIG. 2B is a schematic cross-sectional view of an example of a display apparatus including an organic light-emitting element according to an embodiment of the present disclosure.

FIG. 3 is a schematic view of an example of a display apparatus according to an embodiment of the present disclosure.

FIG. 4A is a schematic view of an example of an imaging apparatus according to an embodiment of the present disclosure.

FIG. 4B is a schematic view of an example of electronic equipment according to an embodiment of the present disclosure.

FIG. 5A is a schematic view of an example of a display apparatus according to an embodiment of the present disclosure.

FIG. 5B is a schematic view of an example of a foldable display apparatus.

FIG. 6A is a schematic view of an example of a lighting apparatus according to an embodiment of the present disclosure.

FIG. 6B is a schematic view of an example of a moving body with a vehicle lamp according to an embodiment of the present disclosure.

FIG. 7A is a schematic view of an example of a wearable device according to an embodiment of the present disclosure.

FIG. 7B is a schematic view of another example of a wearable device according to an embodiment of the present disclosure.

FIG. 8A is a schematic view of an example of an image-forming apparatus according to an embodiment of the present disclosure.

FIG. 8B is a schematic view of an example of an exposure light source for an image-forming apparatus according to an embodiment of the present disclosure.

FIG. 8C is a schematic view of an example of an exposure light source for an image-forming apparatus according to an embodiment of the present disclosure.

### DESCRIPTION OF THE EMBODIMENTS

### <<Organic Compound>>

An organic compound according to the present embodiment is represented by the following general formula [1]:

A-(B)ₘ [1]

An organic compound according to the present embodiment is a compound represented by the following general formula [2-1] or [2-2]. In the general formulae [2-1] and [2-2], the unit A is a portion excluding the unit B. The unit B and R₁ are bonded to a benzene ring (hereinafter sometimes referred to as a “second benzene ring”) bonded to a benzene ring (hereinafter sometimes referred to as a “first benzene ring”) bonded to the triphenylene ring of the unit A.

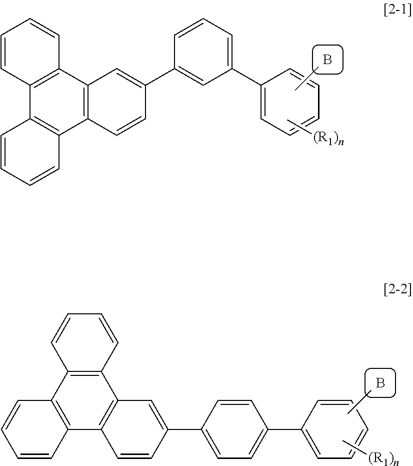

An organic compound according to the present embodiment is an organic compound containing a triphenylene skeleton and a nitrogen-containing chalcogen heterocyclic skeleton with a high T₁ (the lowest excited triplet state). The organic compound represented by the general formula [2-1] advantageously has a higher T₁ than the organic compound represented by the general formula [2-2].

### <R₁>

In the general formulae [2-1] and [2-2], R₁ is independently selected from a deuterium atom, substituted or unsubstituted alkyl groups, substituted or unsubstituted aryl groups, and substituted or unsubstituted heterocyclic groups. When R₁ denotes a substituted or unsubstituted heterocyclic group, R₁ is bonded via a carbon atom. R₁ can be bonded at a position meta to the position at which the first benzene ring and the second benzene ring are bonded.

Examples of the alkyl groups include, but are not limited to, a methyl group, an ethyl group, a n-propyl group, an isopropyl group, a n-butyl group, a tert-butyl group, a sec-butyl group, an octyl group, a cyclohexyl group, a 1-adamantyl group, and a 2-adamantyl group.

Examples of the aryl groups include, but are not limited to, a phenyl group, a biphenyl group, a terphenyl group, a fluorenyl group, and a triphenylenyl group.

The heterocyclic groups can be heteroaryl groups, for example, but are not limited to, a pyridyl group, a pyrimidyl group, a pyrazyl group, a triazolyl group, an oxazolyl group, an oxadiazolyl group, a thiazolyl group, a thiadiazolyl group, a carbazolyl group, a benzofuranyl group, a benzothiophenyl group, a dibenzofuranyl group, and a dibenzothiophenyl group.

The aryl groups and the heterocyclic groups may have a substituent, for example, but not limited to, an alkyl group, such as a methyl group, an ethyl group, a n-propyl group, an isopropyl group, a n-butyl group, a tert-butyl group, a sec-butyl group, an octyl group, a cyclohexyl group, a 1-adamantyl group, or a 2-adamantyl group; an aryl group, such as a phenyl group, a biphenyl group, a terphenyl group, a fluorenyl group, or a triphenylenyl group; or a heterocyclic group, such as a pyridyl group, a pyrimidyl group, a pyrazyl group, a triazolyl group, an oxazolyl group, an oxadiazolyl group, a thiazolyl group, a thiadiazolyl group, a carbazolyl group, a benzofuranyl group, a benzothiophenyl group, a dibenzofuranyl group, or a dibenzothiophenyl group.

<m, n>

m denotes an integer of 1 or more and 5 or less, preferably an integer of 1 or more and 2 or less, more preferably 1. n denotes an integer of 0 or more and 4 or less, preferably an integer of 0 or more and 2 or less, more preferably 0 or more and 1 or less. When m is 2 or more, the units B may be the same or different. When n is 2 or more, R₁s may be the same or different.

### <Unit B>

The unit (B) is a fused ring with a partial structure represented by the following general formula [3-1] or [3-2] or is a combination of a fused ring with a partial structure represented by the general formula [3-1] or [3-2] and a substituted or unsubstituted aromatic ring. The unit B may have one or a plurality of fused rings with a partial structure represented by the general formula [3-1] or [3-2] (hereinafter sometimes referred to as a “fused ring with a partial structure”). The unit B may also have one or a plurality of aromatic rings that form a combination with a fused ring with a partial structure.

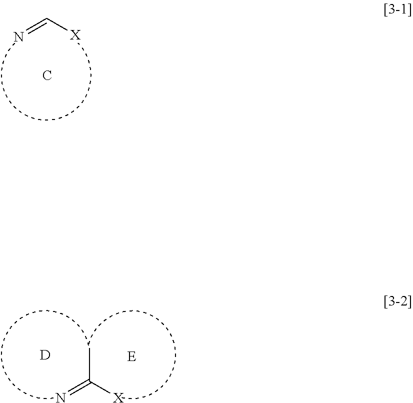

The unit B is bonded to the unit A via a carbon atom. A carbon atom bonded to the unit A may be a carbon atom constituting a fused ring with a partial structure or a carbon atom constituting a substituted or unsubstituted aromatic ring bonded to a fused ring with a partial structure. The unit B can be bonded at a position meta to the position at which the first benzene ring and the second benzene ring are bonded.

[Fused Ring with Partial Structure Represented by General Formula [3-1] or [3-2]]

### {X}

In the general formulae [3-1] and [3-2], X is selected from an oxygen atom, a sulfur atom, a selenium atom, and a tellurium atom. X can be selected from an oxygen atom and a sulfur atom.

### {Rings C to E}

In the general formulae [3-1] and [3-2], rings C to E independently denote a single ring and may have a deuterium atom or a substituted or unsubstituted alkyl group.

The fused ring with the partial structure represented by the general formula [3-1] can be a bicyclic fused ring. The ring C can be a five-membered ring or a heteroaromatic ring. A ring fused with the ring C can be a six-membered ring and can be an aromatic hydrocarbon ring or a heteroaromatic ring.

The fused ring with the partial structure represented by the general formula [3-2] can be a bicyclic or tricyclic fused ring. The ring D can be a six-membered ring or a heteroaromatic ring. The ring E can be a five- or six-membered ring or a non-aromatic heterocyclic or a heteroaromatic ring. When the ring E is a six-membered ring, in addition to X, a chalcogen atom can constitute the ring E. A ring fused with the ring D or E can be a six-membered ring and can be an aromatic hydrocarbon ring or a heteroaromatic ring.

Examples of the alkyl group optionally contained in the rings C to E include, but are not limited to, a methyl group, an ethyl group, a n-propyl group, an isopropyl group, a n-butyl group, a tert-butyl group, a sec-butyl group, an octyl group, a cyclohexyl group, a 1-adamantyl group, and a 2-adamantyl group. The alkyl group may further have a substituent that the aryl group and the heterocyclic group of R₁ may further have.

### SPECIFIC EXAMPLES

Specific examples of the fused ring with a partial structure are described below. As a matter of course, the present disclosure is not limited thereto. These specific examples can satisfy (i) described later.

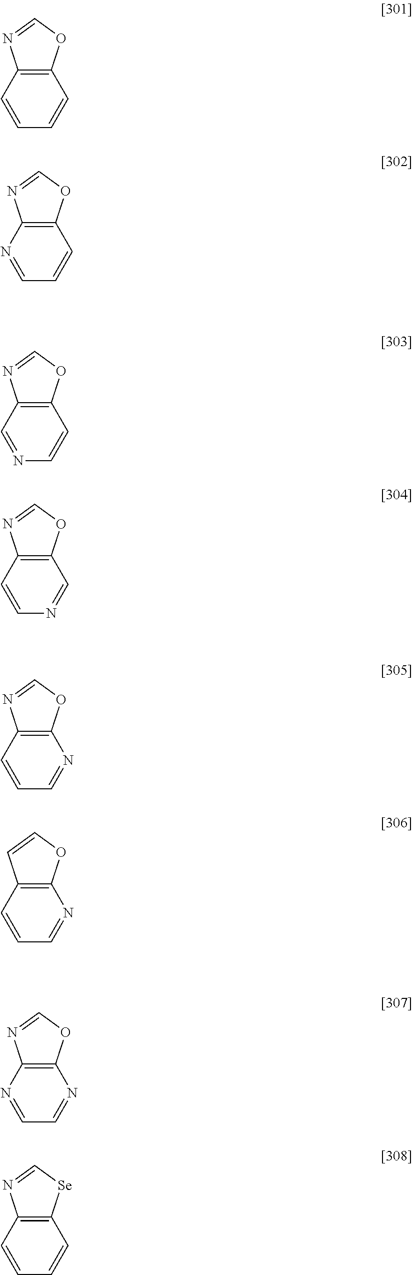

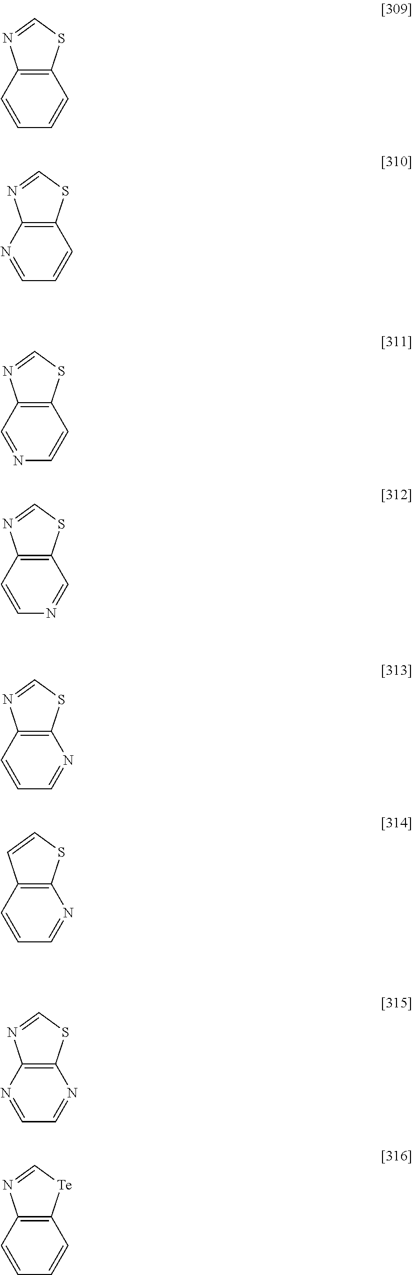

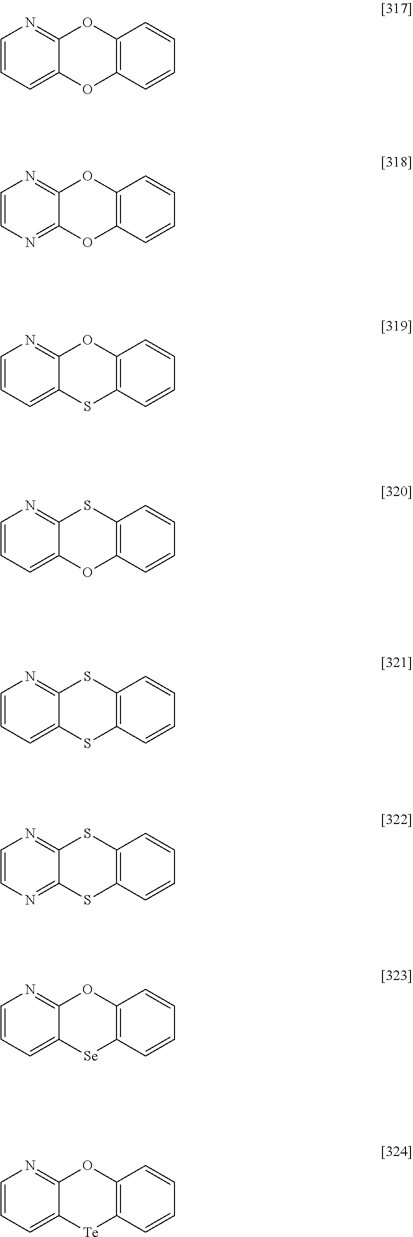

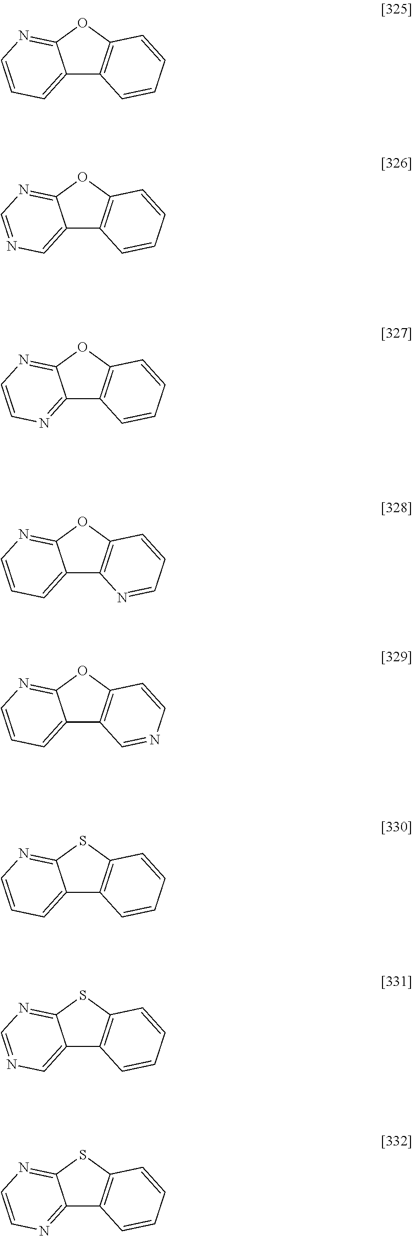

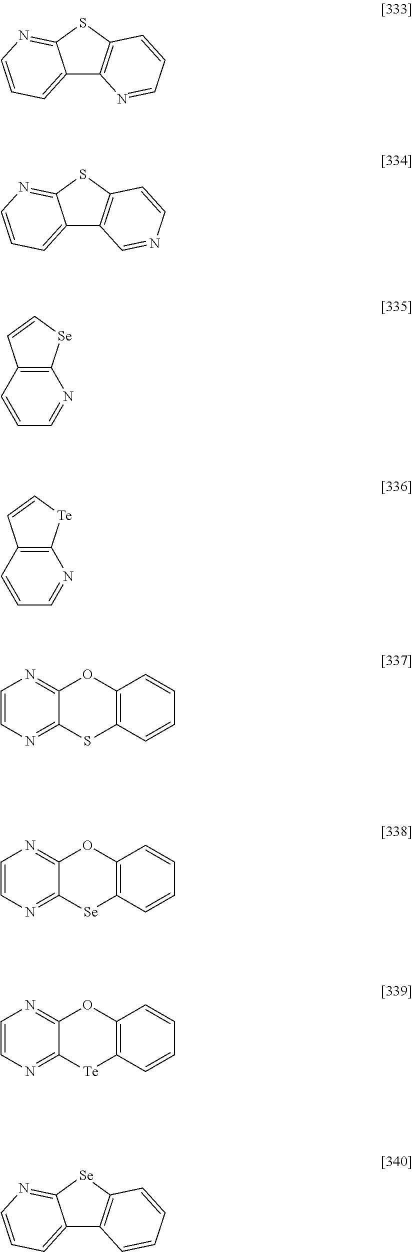

### [Substituted or Unsubstituted Aromatic Ring]

The aromatic ring constituting a combination with the fused ring with a partial structure is an aromatic hydrocarbon ring or a heteroaromatic ring. The aromatic ring can be a single ring or a fused ring with a smaller number of rings to be fused than the fused ring with a partial structure.

Examples of the aromatic hydrocarbon ring include, but are not limited to, a benzene ring, a biphenyl ring, a terphenyl ring, a fluorene ring, and a triphenylene ring.

Examples of the heteroaromatic ring include, but are not limited to, a pyridine ring, a pyrimidine ring, a pyrazine ring, a triazole ring, an oxazole ring, an oxadiazole ring, a thiazole ring, a thiadiazole ring, a carbazole ring, a benzofuran ring, a benzothiophene ring, a dibenzofuran ring, and a dibenzothiophene ring.

The aromatic ring may further have a substituent that the aryl group and the heterocyclic group of R₁ may further have.

<(i) and (ii)>

An organic compound according to the present embodiment satisfies (i) and (ii):
(i) T₁ of the unit A<T₁ of the unit B, and
(ii) highest occupied molecular orbital (HOMO)-lowest unoccupied molecular orbital (LUMO) orbitals responsible for T₁ transition are present in the unit A.

The details are described below.
(i) T₁ of Unit A<T₁ of Unit B

In a light-emitting layer of an organic light-emitting element, particularly when light emission from a triplet excited state is used, the T₁ (the lowest excited triplet state) of a host molecule can be higher than the T₁ of a guest molecule. When the host molecule has a lower T₁, energy is transferred to a lower level, and the existence probability of an excited state in the host molecule increases. This promotes thermal deactivation from T₁ and bond dissociation in the host molecule and reduces light emission efficiency and durability. Thus, to provide an organic light-emitting element with high efficiency and durability, the T₁ of the host molecule can be higher than the T₁ of the guest molecule, and the host molecule can have a much higher T₁.

In organic phosphorescent elements, particularly in green phosphorescent elements, a triphenylene skeleton is widely used. This is due to S₁ (the lowest excited singlet state), T₁, and thermal stability suitable for a green phosphorescent element. The present inventors have found that the T₁ of the unit B higher than the T₁ of the unit A results in a higher T₁ of the entire molecule.

Table 1 shows calculated T₁s (peak wavelength: nm) of fused rings. In Table 1, “Basic skeleton (present disclosure)” shows a triphenylene ring and fused rings satisfying (i), and “Basic skeleton (comparison)” shows fused rings not satisfying (i). Table 1 shows that the fused rings [301], [309], [318], [322], and [330] with a partial structure have a higher T₁ than the triphenylene skeleton. The T is of the units A and B tend to depend on the T₁ of the fused ring with the largest number of rings to be fused. Thus, an organic compound according to the present embodiment has a high T₁ and is suitable for an organic light-emitting element with high efficiency and durability.

TABLE 1

| Basic skeleton (present | T1 | Basic skeleton (for | T1 |
|---|---|---|---|
| disclosure) | (calc.) | comparison) | (calc.) |
| 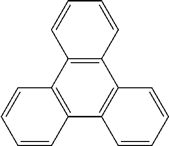 [SMILES: c1ccc2c(c1)c1ccccc1c1ccccc21] | 434 | 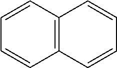 | 454 |
| 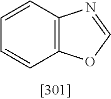 | 351 | 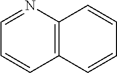 | 438 |
| 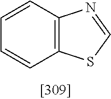 | 370 | 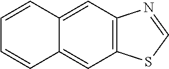 [SMILES: c1ccc2cc3scnc3cc2c1] | 522 |
| 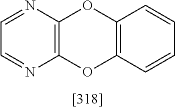 [SMILES: c1ccc2c(c1)Oc1nccnc1O2] | 405 | 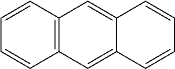 [SMILES: c1ccc2cc3ccccc3cc2c1] | 688 |
| 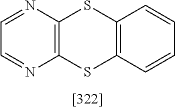 [SMILES: c1ccc2c(c1)Sc1nccnc1S2] | 398 | 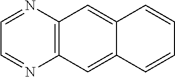 [SMILES: c1ccc2cc3nccnc3cc2c1] | 687 |
| 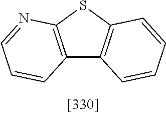 [SMILES: c1ccc2c(c1)sc1ncccc12] | 394 | 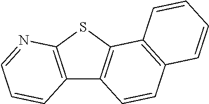 [SMILES: c1ccc2c(c1)ccc1c3cccnc3sc21] | 472 |

(ii) HOMO-LUMO orbitals responsible for T₁ transition are present in unit A

A compound in an organic layer, particularly a light-emitting layer, of an organic light-emitting element transits repeatedly between the ground state and the excited state in the process of light emission of the organic light-emitting element. In particular, in an organic phosphorescent element, it is important to control the lowest excited triplet state (T₁), which occupies 75% of the excited state. For example, energy can be efficiently transferred from the T₁ of a host molecule to a guest molecule to efficiently emit light from the guest molecule. In this process, when the host molecule has a reactive T₁, the excitation energy is used for a reaction with an adjacent molecule, which reduces energy transfer efficiency and durability due to the generation of a quencher molecule.

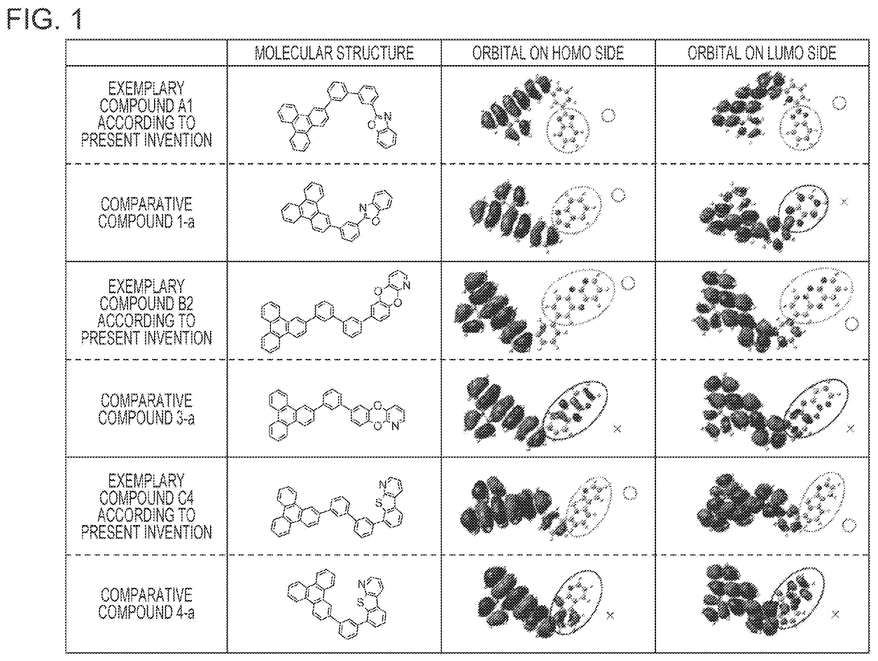

A heterocycle has a heteroatom in the skeleton and tends to have high polarization. Thus, when a heterocycle has excitation energy, the excitation energy is polarized and localized and thereby increases the reactivity with an adjacent molecule. Thus, the present inventors paid attention to the fact that the unit B has no HOMO-LUMO orbitals responsible for T₁ transition of a host molecule. In other words, the unit A has all the HOMO-LUMO orbitals responsible for the T₁ transition of the host molecule. FIG. 1 shows distribution diagrams of calculated HOMO-LUMO orbitals responsible for T₁ transition of each of an exemplary compound A1 and a comparative compound 1-a, an exemplary compound B2 and a comparative compound 3-a, and an exemplary compound C4 and a comparative compound 4-a of the present embodiment.

The present inventors have found that the unit A has all the HOMO-LUMO orbitals responsible for T₁ transition of a host molecule when the unit B is connected to the triphenylene skeleton via two phenylene spacers. Thus, an organic compound according to the present embodiment has T₁ transition on the stable triphenylene skeleton, has high energy transfer efficiency to a guest molecule, has fewer quencher molecules, and has high durability.

### <Other Characteristics>

An organic compound according to the present embodiment has the following characteristics in addition to (i) and (ii) described above.
(iii) The unit B has a nitrogen-carbon-chalcogen atom skeleton (N═C—X).
(iv) There is no bond with low bond energy.
(v) The unit B has at least one fused bicyclic or polycyclic heterocycle.

The details are described below.
(iii) The unit B has a nitrogen-carbon-chalcogen atom skeleton (N═C—X).

In (ii), it has been described that in an organic phosphorescent element energy can be efficiently transferred from the T₁ of a host molecule to a guest molecule to efficiently emit light from the guest molecule. An energy transfer process of triplet energy is known to occur by Dexter energy transfer. To improve the efficiency of Dexter energy transfer, it is important to minimize the distance between a host molecule and a guest molecule. As a result of intensive studies, the present inventors have found that, to reduce the distance between a host molecule and a guest molecule, it is effective to have at least one partial structure represented by the general formula [3-1] or [3-2], particularly a nitrogen-carbon-chalcogen atom skeleton (N═C—X). It is known that, when the guest molecule is a metal complex, a nitrogen-carbon-chalcogen atom skeleton (N═C—X) is suitably coordinated to a metal atom (for example, an iridium atom) of the guest molecule. The use of a compound with a nitrogen-carbon-chalcogen atom skeleton (N═C—X) arranged as described below as a host molecule increases the intermolecular interaction with the guest molecule and reduces the intermolecular distance from the guest molecule.

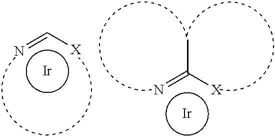

Table 2 shows the external quantum efficiency (E.Q.E) ratio and the durability ratio (luminance decay rate ratio) of an exemplary compound A13 of the present embodiment and a comparative compound 5-a without a partial structure represented by the general formula [3-1] or [3-2]. The details are described in Exemplary Embodiment 27 and Comparative Example 5. The E.Q.E (efficiency) ratio and durability ratio in Exemplary Embodiment 27 are set to 1.0 as reference. It can be seen that the exemplary compound A13 has higher efficiency and durability than the comparative compound 5-a.

TABLE 2

| | | | E.Q.E. (efficiency) | Durability ratio |
|---|---|---|---|---|
| | | | ratio described in | described in |
| | Host | Guest | exemplary | exemplary |
| | molecule | molecule | embodiment | embodiment |
| Exemplary compound A13 according to present disclosure | 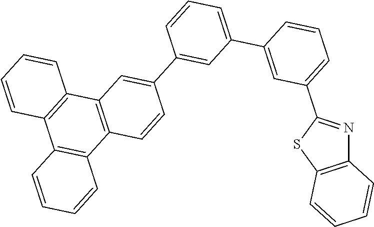 [SMILES: c1cc(-c2cccc(-c3nc4ccccc4s3)c2)cc(-c2ccc3c4ccccc4c4ccccc4c3c2)c1] | Ir (ppy) 3 | 1.0 (Exemplary embodiment 27) | 1.0 (Exemplary embodiment 27) |
| Comparative compound 5-a | 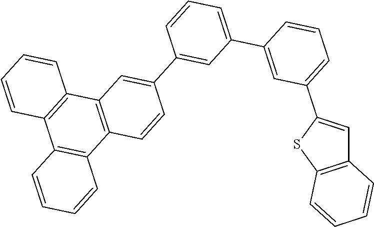 [SMILES: c1cc(-c2cccc(-c3cc4ccccc4s3)c2)cc(-c2ccc3c4ccccc4c4ccccc4c3c2)c1] | Ir (ppy) 3 | 0.9 (Comparative example 5) | 0.9 (Comparative example 5) |

(iv) There is no bond with low bond energy.

A compound in an organic layer, particularly a light-emitting layer, of an organic light-emitting element transits repeatedly between the ground state and the excited state in the process of light emission of the organic light-emitting element. In this process, intense motions, such as expansion and contraction and rotation, of molecules occur. At this time, an easily dissociable bond, if present, may be cleaved, and the compound may be partially separated. When the compound is partially separated, the compound has a changed structure. Thus, an easily separable compound has low durability. When such a compound is used for an organic light-emitting element, the separated portion acts as a quencher and reduces the durability of the element. Thus, a molecule with a structure without easily dissociable bonds and resistant to separation has high durability.

Table 3 shows the bond dissociation energy, the E.Q.E (efficiency) ratio described in the exemplary embodiments, and the durability ratio (luminance decay rate ratio) described in the exemplary embodiments for the exemplary compound A13 and a comparative compound 2-b, which is related to the description of Patent Literature 2. The E.Q.E (efficiency) ratio and durability ratio in Exemplary Embodiment 27 are set to 1.0 as reference. The bond dissociation energy is the lowest bond energy of single bonds in each molecule. The comparative compound 2-b has a freely rotatable C—N bond and therefore has a low bond energy of 64 kcal/mol. In contrast, the exemplary compound A13 has only freely rotatable bonds composed of a C—C bond and has a high bond energy of 84 kcal/mol.

Thus, an organic compound according to the present embodiment is less likely to be separated by bond cleavage and has higher durability than the comparative compound 2-b. Thus, when a compound according to the present embodiment is used for an organic layer of an organic light-emitting element, the compound is rarely separated by bond cleavage during the operation of the element. Thus, the organic light-emitting element is less likely to be deteriorated even during a long-term operation and can have high durability.

TABLE 3

| | | | E.Q.E. | |
|---|---|---|---|---|
| | | | (efficiency) ratio | Durability ratio |
| | | Bond | described in | described in |
| | | dissociation | exemplary | exemplary |
| | Host molecule | energy | embodiment | embodiment |
| Exemplary compound A13 according to present disclosure | 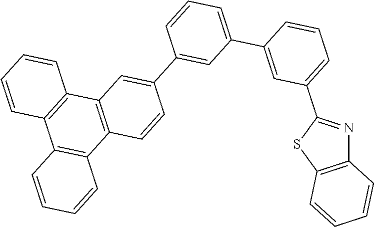 [SMILES: c1cc(-c2cccc(-c3nc4ccccc4s3)c2)cc(-c2ccc3c4ccccc4c4ccccc4c3c2)c1] | C-C 84 kcal/mol | 1.0 (Exemplary embodiment 27) | 1.0 (Exemplary embodiment 27) |
| Comparative compound 2-b | 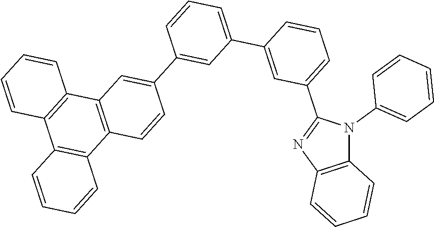 [SMILES: c1ccc(-n2c(-c3cccc(-c4cccc(-c5ccc6c7ccccc7c7ccccc7c6c5)c4)c3)nc3ccccc32)cc1] | C-N 64 kcal/mol | 0.9 (Comparative example 2) | 0.7 (Comparative example 2) |

(v) The unit B has at least one fused bicyclic or polycyclic heterocycle.

High thermal stability, for example, a high glass transition temperature (Tg), is suitable for an organic light-emitting element. This is because a high Tg reduces the generation of crystal grain boundaries, trap levels, and quenchers associated with fine crystallization even during the operation of the element, and high carrier transport ability and efficient emission properties can be maintained. Consequently, an organic light-emitting element with high durability and efficiency can be provided.

Table 4 shows the evaluation results of Tg of the exemplary compound A13 and a comparative compound 6-a by differential scanning calorimetry (DSC) measurement. It can be said that a higher glass transition temperature results in improved amorphous properties and thermal stability. The Tg is preferably 100° C. or more, more preferably 120° C. or more, still more preferably 130° C. or more. In Table 4, Tg of 100° C. or more is good “O”, and Tg of less than 100° C. is poor “x”. In the DSC measurement, the glass transition temperature was measured by rapidly cooling approximately 2 mg of a sample sealed in an aluminum pan from a high temperature exceeding the melting point to bring the sample into an amorphous state and then heating the sample at a heating rate of 20° C./min. DSC 204 F1 manufactured by NETZSCH was used as a measuring apparatus.

Table 4 also shows the E.Q.E (efficiency) ratio and the durability ratio (luminance decay rate ratio) described in the exemplary embodiments. The E.Q.E (efficiency) ratio and durability ratio in Exemplary Embodiment 27 are set to 1.0 as reference.

The comparative compound 6-a had a low Tg of less than 100° C. and a durability ratio of 0.6. In contrast, the exemplary compound A13 had a Tg of 100° C. or more and high durability. Thus, an organic compound according to the present embodiment has good amorphous properties and higher thermal stability. Thus, a stable amorphous film can be maintained even during the operation of the element, and a long-life organic light-emitting element can be provided.

TABLE 4

| | | | E.Q.E. (efficiency) | Durability ratio |
|---|---|---|---|---|
| | | | ratio described in | described in |
| | Host | Thermal | exemplary | exemplary |
| | molecule | stability | embodiment | embodiment |
| Exemplary compound A13 according to present disclosure | 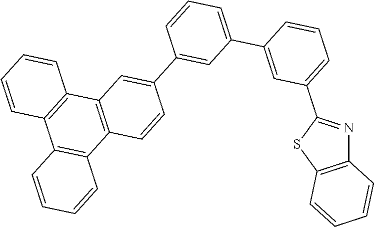 [SMILES: c1cc(-c2cccc(-c3nc4ccccc4s3)c2)cc(-c2ccc3c4ccccc4c4ccccc4c3c2)c1] | ○ | 1.0 (Exemplary embodiment 27) | 1.0 (Exemplary embodiment 27) |
| Comparative compound 6-a | 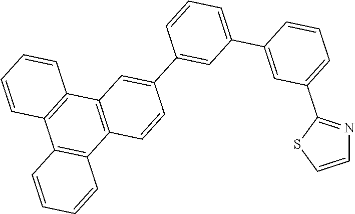 [SMILES: c1cc(-c2cccc(-c3nccs3)c2)cc(-c2ccc3c4ccccc4c4ccccc4c3c2)c1] | x | 0.8 (Comparative example 6) | 0.6 (Comparative example 6) |

An organic compound according to the present embodiment can have the following characteristics.
(vi) In the general formula [2-1], R₁ and the unit B are bonded only at a position meta to the position at which the first benzene ring and the second benzene ring are bonded.

The details are described below.
(vi) In the general formula [2-1], R₁ and the unit B are bonded only at a position meta to the position at which the first benzene ring and the second benzene ring are bonded.

In the general formula [2-1], R₁ and the unit B can be bonded only at a position meta to the position at which the first benzene ring and the second benzene ring are bonded. Thus, a linking group (the first benzene ring and the second benzene ring) between the triphenylene in the unit A and the unit B is composed only of a phenylene group bonded at the meta position. A phenylene group bonded at the meta position has little influence on the conjugation length, and a high T₁ can be maintained. A phenylene group bonded at the meta position can improve the film properties of an organic film formed by vapor deposition or the like. More specifically, it improves the glass transition temperature and heat resistance. This improves the life of the element.

### SPECIFIC EXAMPLES

Specific structural formulae of an organic compound according to the present embodiment are exemplified below, but, as a matter of course, the present disclosure is not limited thereto.

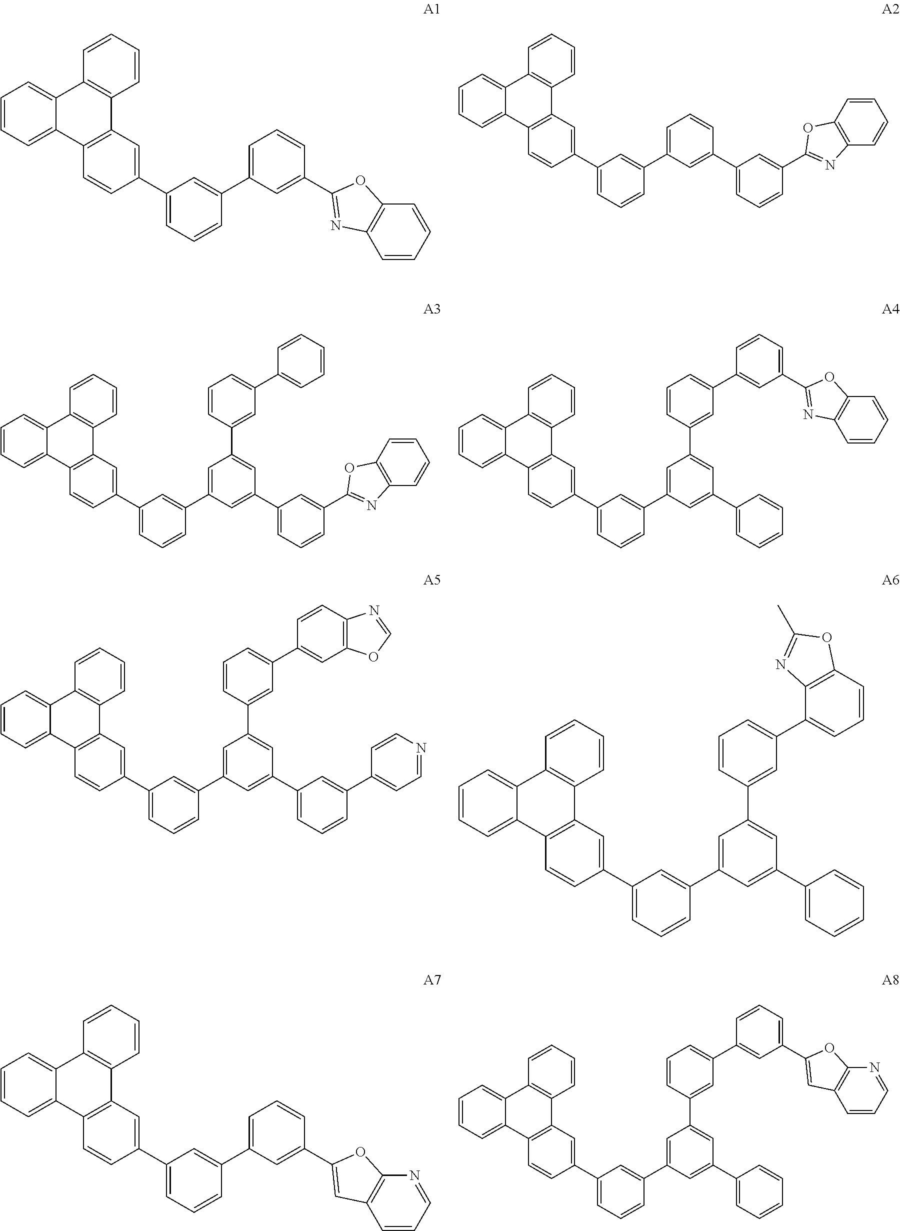

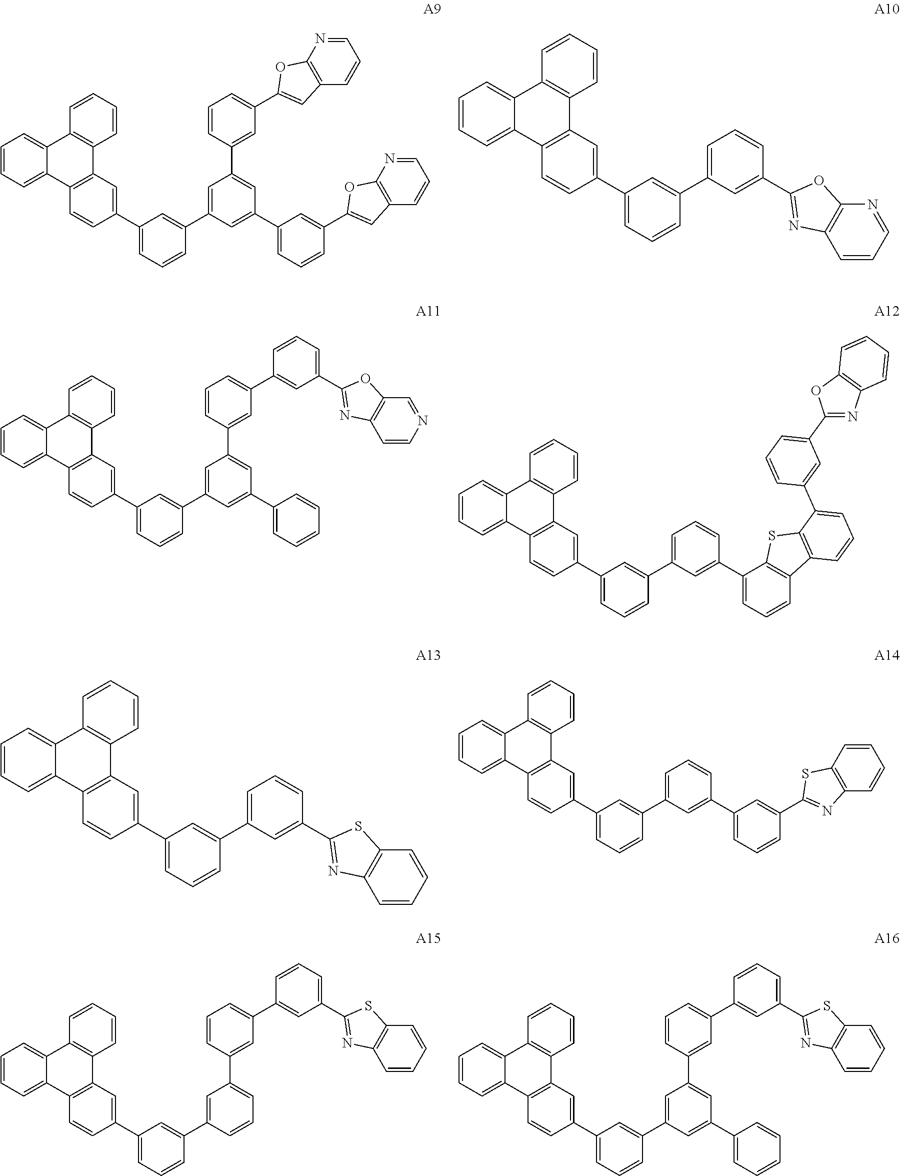

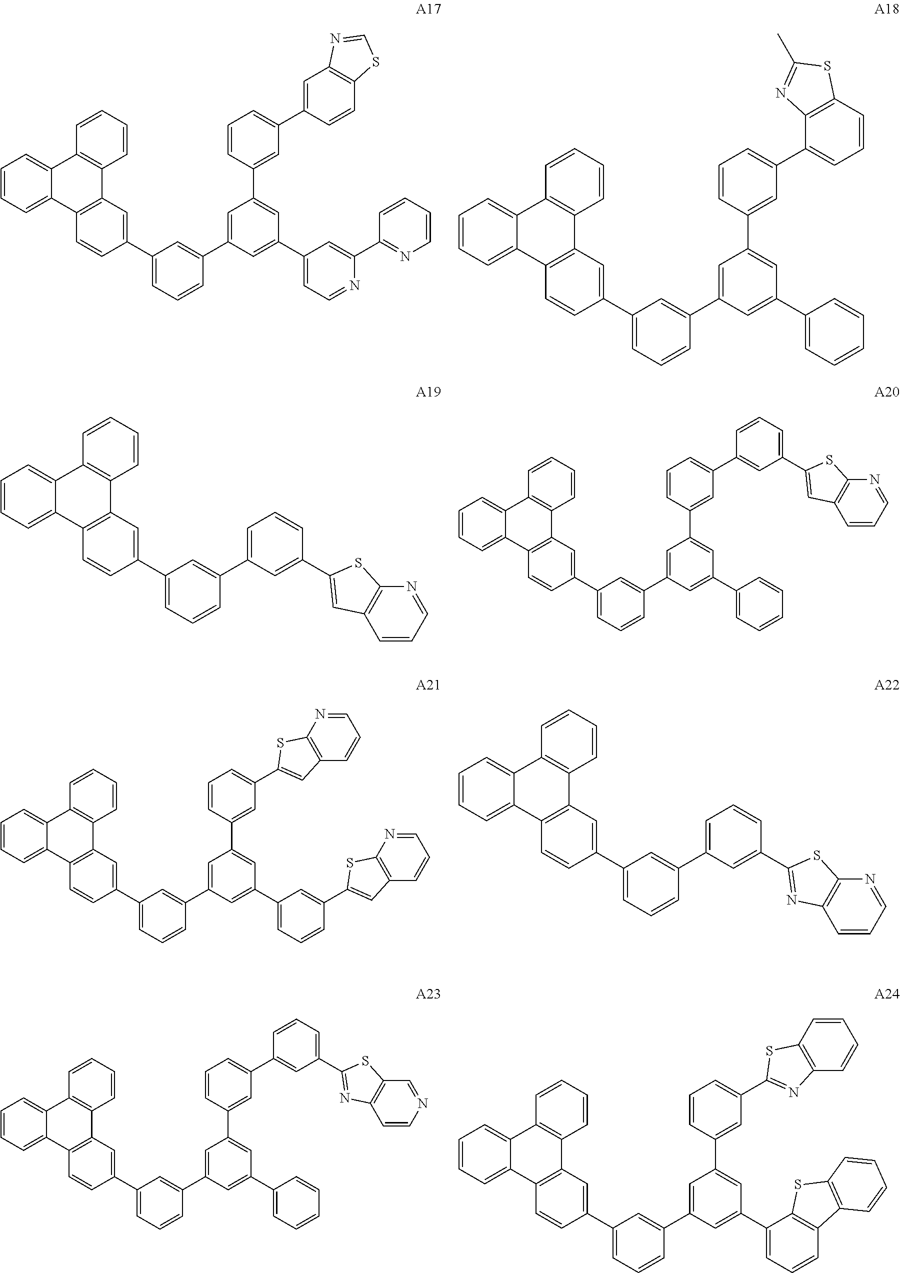

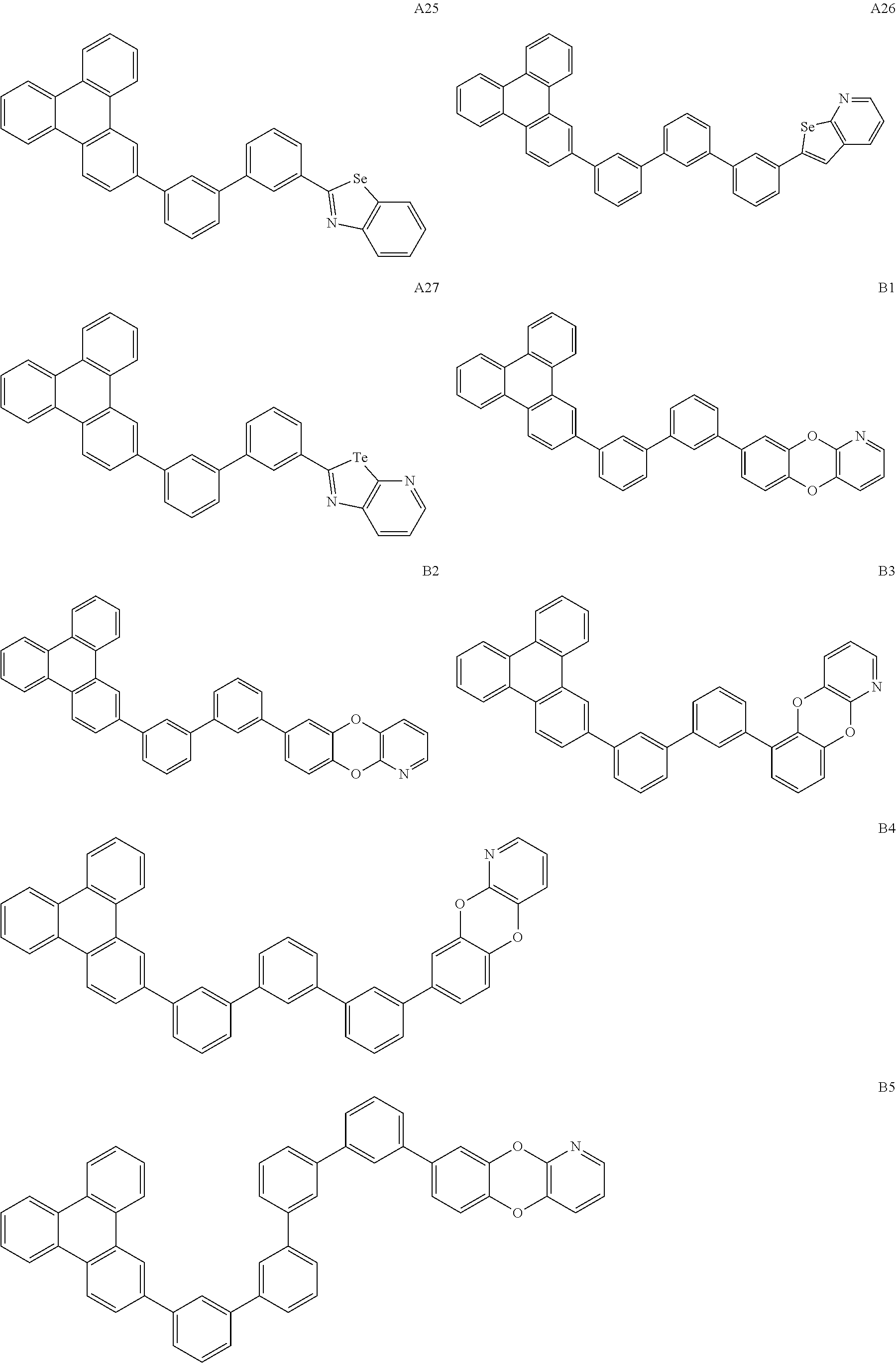

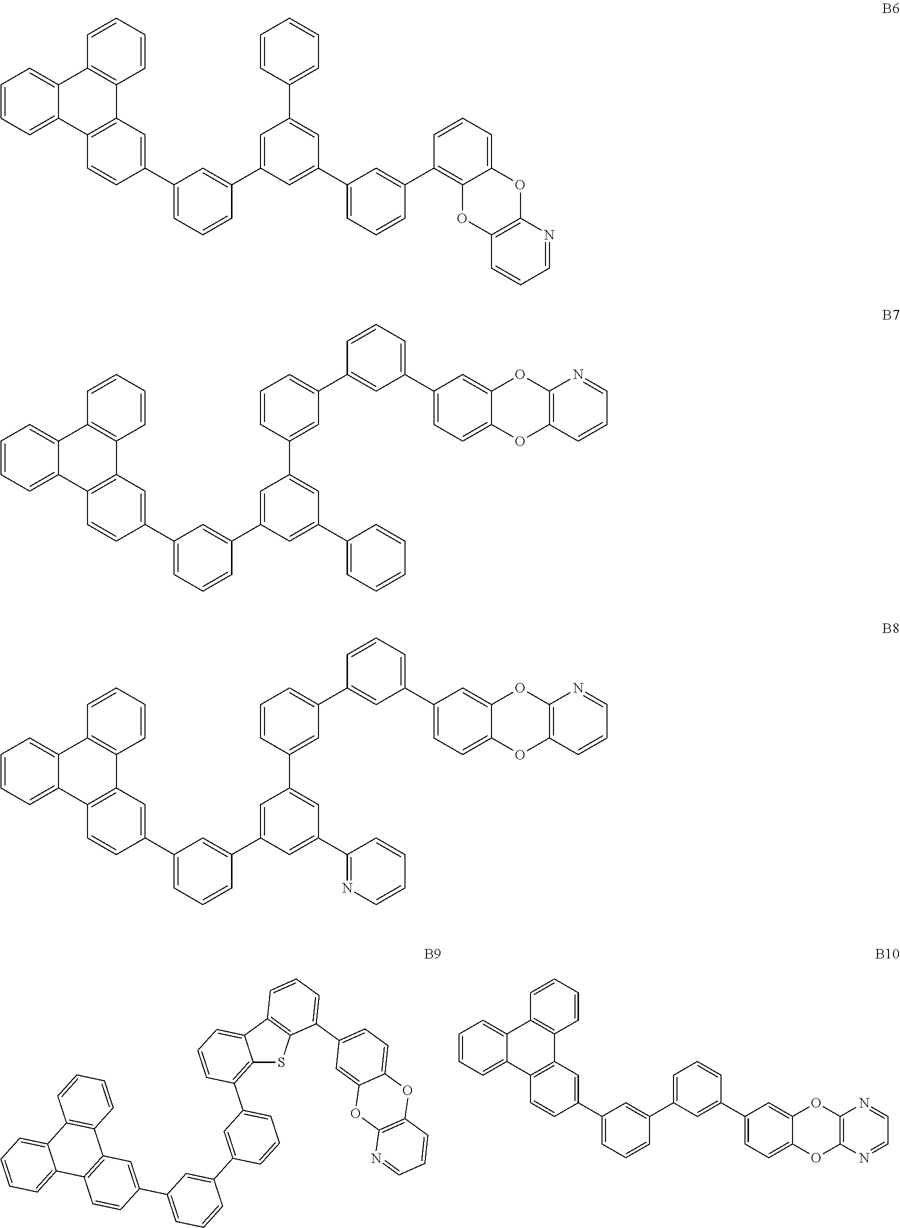 [SMILES: c1ccc(-c2cc(-c3cccc(-c4ccc5c6ccccc6c6ccccc6c5c4)c3)cc(-c3cccc(-c4cccc5c4Oc4cccnc4O5)c3)c2)cc1 ; c1ccc(-c2cc(-c3cccc(-c4cccc(-c5ccc6c(c5)Oc5ncccc5O6)c4)c3)cc(-c3cccc(-c4ccc5c6ccccc6c6ccccc6c5c4)c3)c2)cc1 ; c1ccc(-c2cc(-c3cccc(-c4cccc(-c5ccc6c(c5)Oc5ncccc5O6)c4)c3)cc(-c3cccc(-c4ccc5c6ccccc6c6ccccc6c5c4)c3)c2)nc1 ; c1cc(-c2cccc(-c3cccc4c3sc3c(-c5ccc6c(c5)Oc5ncccc5O6)cccc34)c2)cc(-c2ccc3c4ccccc4c4ccccc4c3c2)c1 ; c1cc(-c2cccc(-c3ccc4c5ccccc5c5ccccc5c4c3)c2)cc(-c2ccc3c(c2)Oc2nccnc2O3)c1]

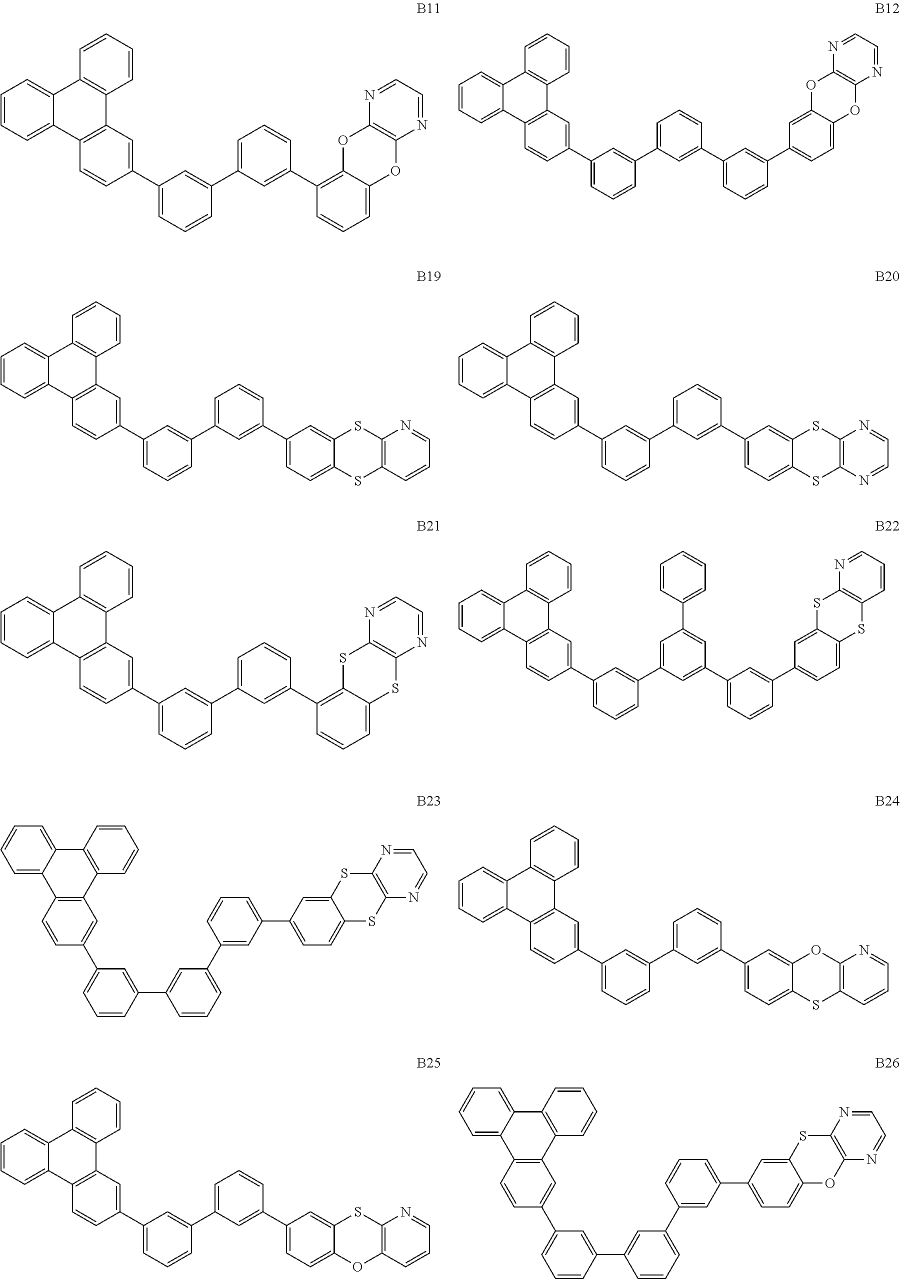

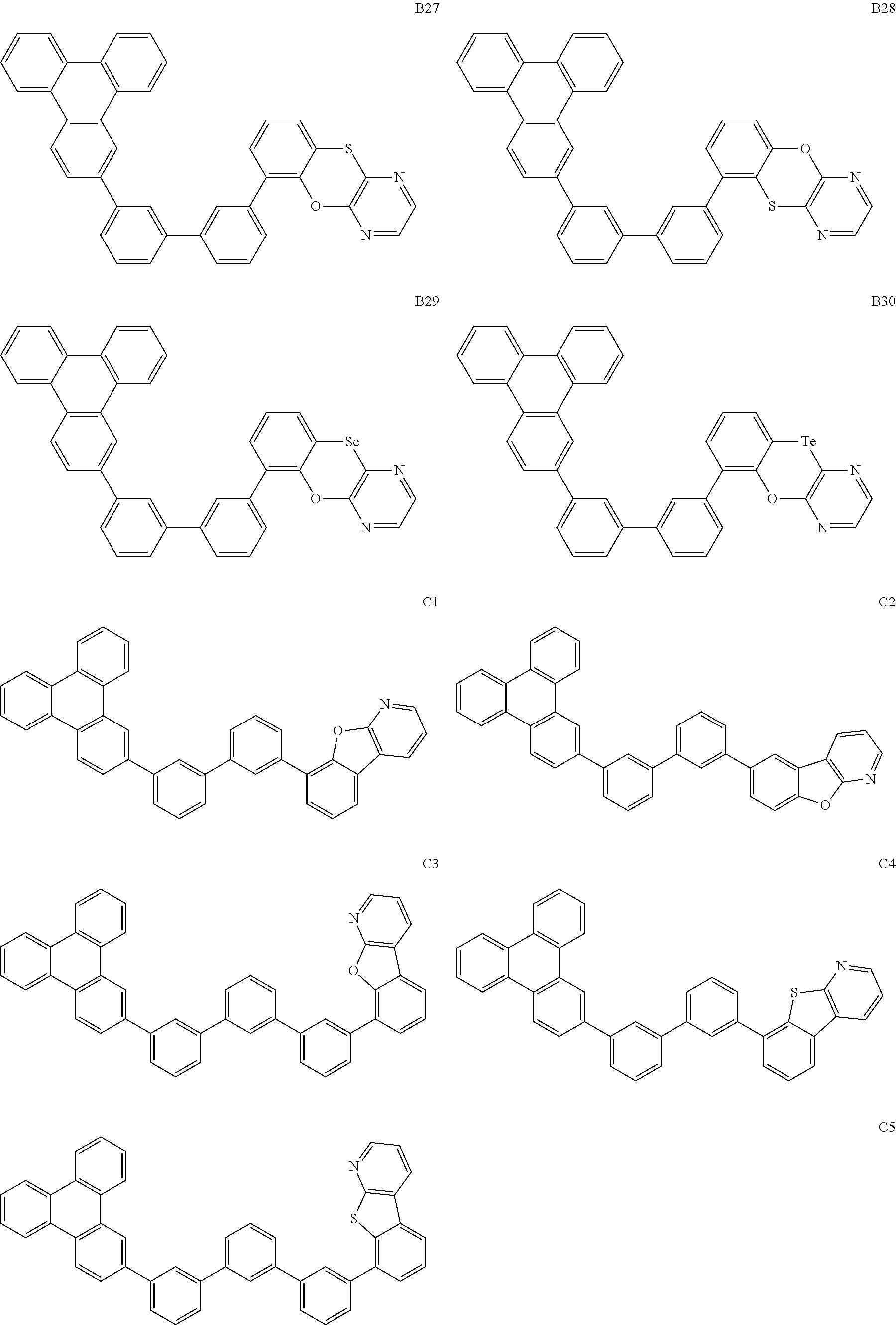

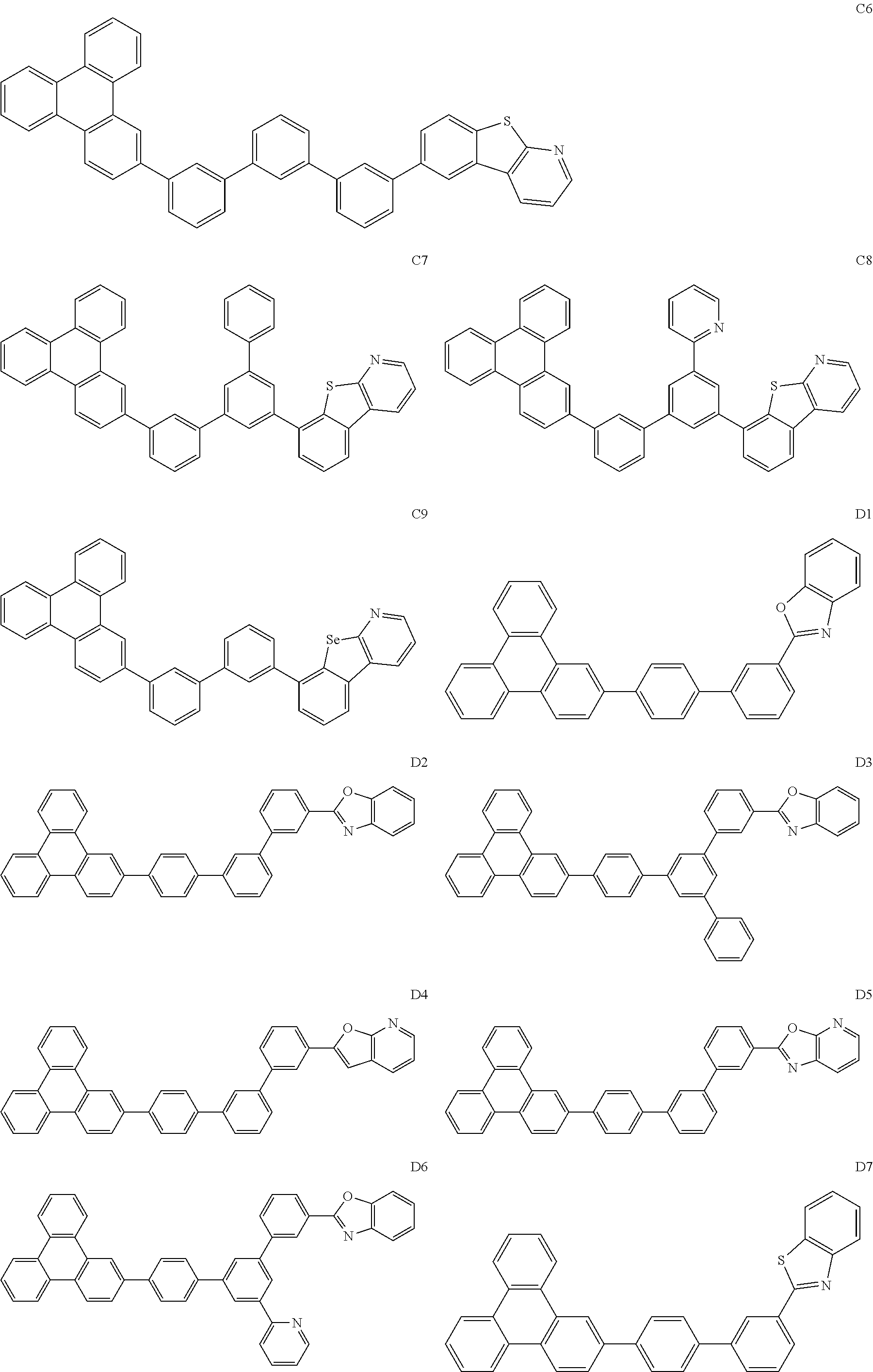

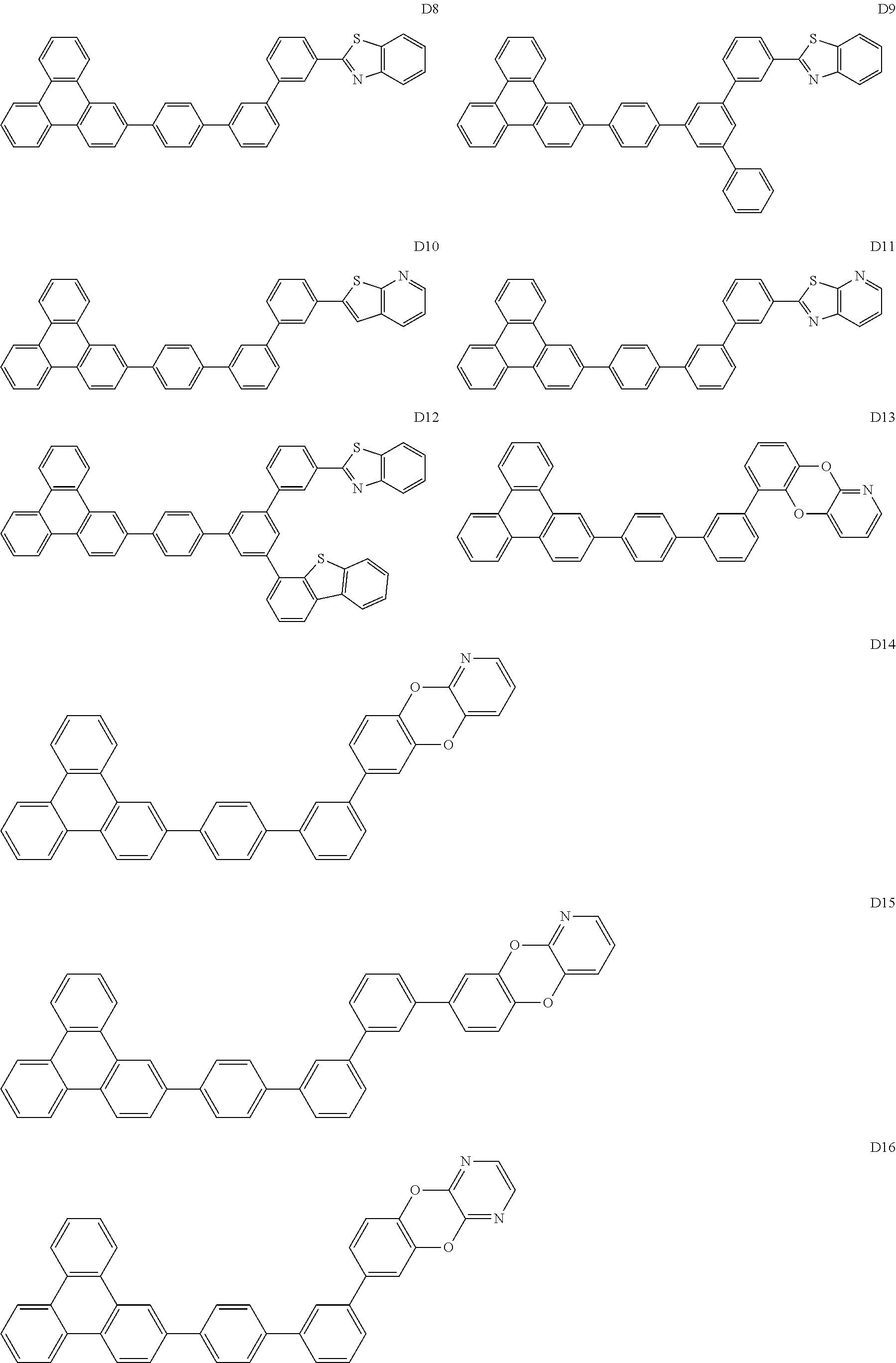

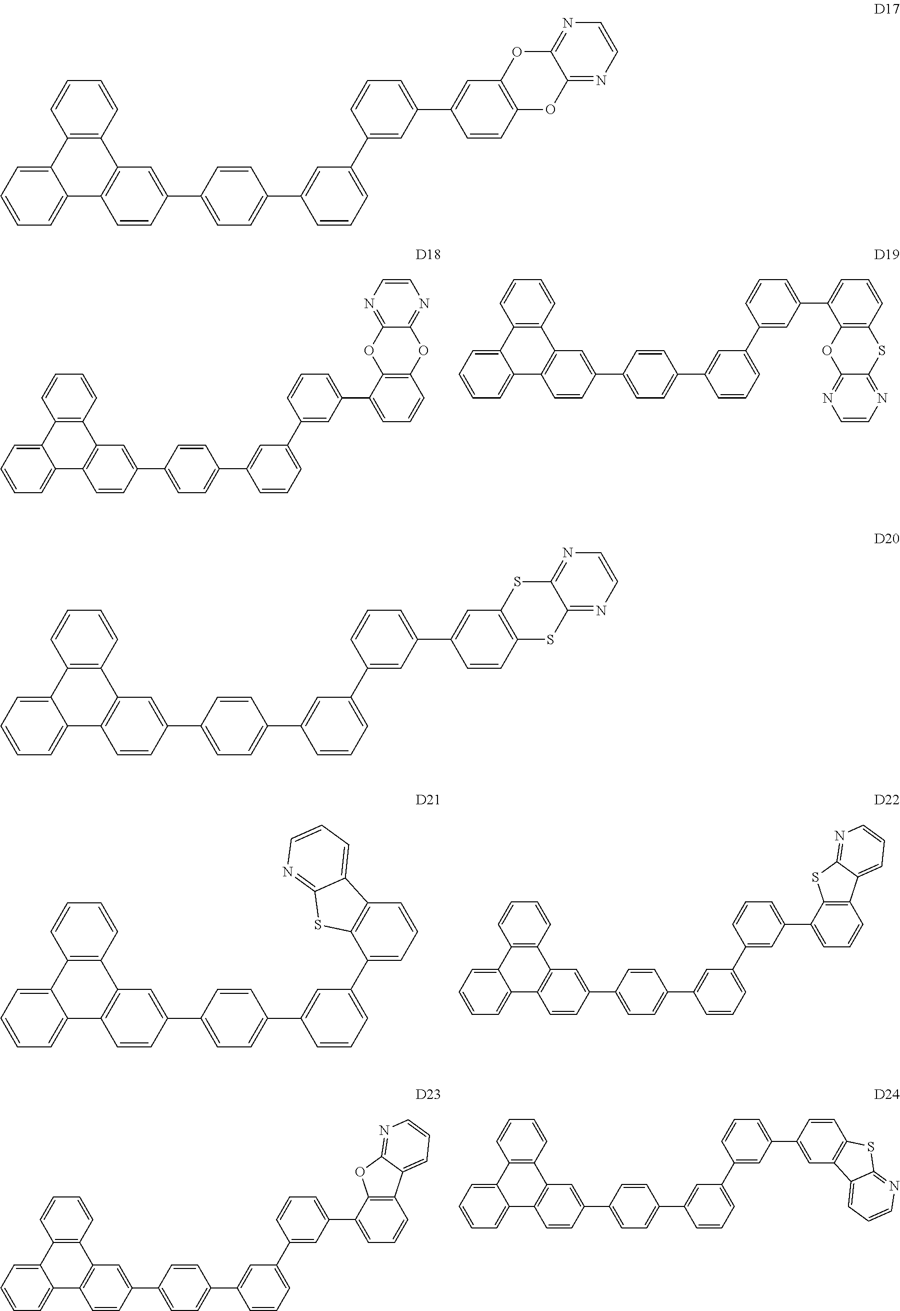

An exemplary compound belonging to the group A is a compound represented by the formula [2-1] in which the fused ring with a partial structure in the unit B is a bicyclic fused ring of a five-membered ring and a six-membered ring. The group A is a group of compounds with a low molecular weight and high sublimability among the compounds according to the present embodiment.

An exemplary compound belonging to the group B is a compound represented by the formula [2-1] in which the fused ring with a partial structure in the unit B is a fused ring of three six-membered rings and contains two chalcogen atoms. The compounds belonging to the group B have a higher T₁ among the compounds according to the present embodiment and, due to the two chalcogen atoms, have a strong interaction with an Ir complex. Thus, the group B is a group of compounds with higher efficiency and longer life when used for an organic light-emitting element.

An exemplary compound belonging to the group C is a compound represented by the formula [2-1] in which the fused ring with a partial structure in the unit B is a tricyclic fused ring of a six-membered ring, a five-membered ring, and a six-membered ring. The compounds belonging to the group C can have both a high T₁ and good film properties among the compounds according to the present embodiment. Thus, the group C is a group of compounds with longer life when used for an organic light-emitting element.

Exemplary compounds belonging to the group D are compounds represented by the formula [2-2]. The compounds belonging to the group D have a smaller band gap among the compounds according to the present embodiment. Thus, the group D is a group of compounds with low-voltage driving characteristics when used for an organic light-emitting element.

### <<Organic Light-Emitting Element>>

The organic light-emitting element according to the present embodiment includes a pair of electrodes and an organic compound layer between the pair of electrodes. When the organic compound layer is a laminate of a plurality of layers, the organic compound layer may have a hole-injection layer, a hole-transport layer, an electron-blocking layer, a hole/exciton-blocking layer, an electron-transport layer, and/or an electron-injection layer, in addition to the light-emitting layer. The light-emitting layer may be a single layer or a laminate of a plurality of layers.

In the organic light-emitting element according to the present embodiment, at least one layer of the organic compound layers contains an organic compound according to the present embodiment. More specifically, an organic compound according to the present embodiment is contained in one of the light-emitting layer, the hole-injection layer, the hole-transport layer, the electron-blocking layer, the hole/exciton-blocking layer, the electron-transport layer, the electron-injection layer, and the like. An organic compound according to the present embodiment can be contained in the light-emitting layer.

In the organic light-emitting element according to the present embodiment, when an organic compound according to the present embodiment is contained in the light-emitting layer, the light-emitting layer may be composed only of the organic compound according to the present embodiment or may be composed of the organic compound according to the present embodiment and another compound. When the light-emitting layer is composed of an organic compound according to the present embodiment and another compound, the organic compound according to the present embodiment may be used as a host or a guest of the light-emitting layer. The compound may also be used as an assist material that may be contained in the light-emitting layer.

The host is a compound with the highest mass ratio among the compounds constituting the light-emitting layer. The guest is a compound that has a lower mass ratio than the host among the compounds constituting the light-emitting layer and that is a principal light-emitting compound. The assist material is a compound that has a lower mass ratio than the host among the compounds constituting the light-emitting layer and that assists the guest in emitting light. The assist material is also referred to as a second host.

When an organic compound according to the present embodiment is used as a host in a light-emitting layer, the concentration of the host is preferably 50% by mass or more and 99% by mass or less, more preferably 70% by mass or more and 99% by mass or less, of the entire light-emitting layer.

The present inventors have conducted various studies and have found that an organic compound according to the present embodiment can be used as a host or an assist in a light-emitting layer, particularly as a host in the light-emitting layer, to provide an element that can efficiently emit bright light and that has very high durability. The light-emitting layer may be of monolayer or multilayer or may have a mixture of colors by containing a light-emitting material of another emission color. The term “multilayer”, as used herein, refers to a laminate of a light-emitting layer and another light-emitting layer. In such a case, the emission color of the organic light-emitting element is not particularly limited. More specifically, the emission color may be white or a neutral color. For white emission color, for example, when the light-emitting layer has a blue emission color, another light-emitting layer has an emission color different from blue, that is, green or red. Such a layer is formed by vapor deposition or coating. This is described in detail below in exemplary embodiments.

The organic light-emitting element according to the present embodiment can have a pair of electrodes and a light-emitting layer between the pair of electrodes, wherein the light-emitting layer has at least a first organic compound and a phosphorescent organometallic complex, and the first organic compound is an organic compound according to the present embodiment represented by the general formula [1].

A specific element structure of the organic light-emitting element according to the present embodiment may be a multilayer element structure including an electrode layer and an organic compound layer shown in the following (1) to (6) sequentially stacked on a substrate. In any of the element structures, the organic compound layer always includes a light-emitting layer containing a light-emitting material.
(1) positive electrode/light-emitting layer/negative electrode
(2) positive electrode/hole-transport layer/light-emitting layer/electron-transport layer/negative electrode
(3) positive electrode/hole-transport layer/light-emitting layer/electron-transport layer/electron-injection layer/negative electrode
(4) positive electrode/hole-injection layer/hole-transport layer/light-emitting layer/electron-transport layer/negative electrode
(5) positive electrode/hole-injection layer/hole-transport layer/light-emitting layer/electron-transport layer/electron-injection layer/negative electrode
(6) positive electrode/hole-transport layer/electron-blocking layer/light-emitting layer/hole-blocking layer/electron-transport layer/negative electrode

However, these element structure examples are only very basic element structures, and the present disclosure is not limited to these structures. Various layer structures are possible; for example, an insulating layer, an adhesive layer, or an interference layer is formed at an interface between an electrode and an organic compound layer, an electron-transport layer or a hole-transport layer is composed of two layers with different ionization potentials, or a light-emitting layer is composed of two layers formed of different light-emitting materials.

Among the element structures shown in (1) to (6), the structure (6) has both an electron-blocking layer and a hole-blocking layer. Thus, the electron-blocking layer and the hole-blocking layer in (6) can securely confine both carriers of holes and electrons in the light-emitting layer. Thus, the organic light-emitting element has no carrier leakage and high light emission efficiency.

The mode (element form) of extracting light from a light-emitting layer may be a bottom emission mode of extracting light from an electrode on the substrate side or a top emission mode of extracting light from the side opposite to the substrate side. The mode may also be a double-sided extraction mode of extracting light from the substrate side and from the side opposite to the substrate side.

In the organic light-emitting element according to the present embodiment, an organic compound according to the present embodiment can be contained in the light-emitting layer of the organic compound layer. The light-emitting layer contains at least a phosphorescent organometallic complex. The use of a compound contained in the light-emitting layer depends on the concentration of the compound in the light-emitting layer. More specifically, the compound is divided into a main component and an auxiliary component depending on the concentration of the compound in the light-emitting layer.

A compound serving as a main component is a compound with the maximum mass ratio (content) among the compound group contained in the light-emitting layer and is also referred to as a host. The host is a compound that is present as a matrix around the light-emitting material in the light-emitting layer and that is mainly responsible for carrier transport to the light-emitting material and excitation energy supply to the light-emitting material.

A compound serving as an auxiliary component is a compound other than the main component and can be referred to as a guest (dopant), a light-emitting assist material, or a charge injection material depending on the function of the compound. A guest, which is an auxiliary component, is a compound (light-emitting material) responsible for main light emission in the light-emitting layer. A light-emitting assist material, which is an auxiliary component, is a compound that assists the light emission of the guest and that has a lower mass ratio (content) than the host in the light-emitting layer. The light-emitting assist material is also referred to as a second host because of its function.

The concentration of the guest is 0.01% by mass or more and 50% by mass or less, preferably 0.1% by mass or more and 20% by mass or less, of the total amount of the constituent materials of the light-emitting layer. The concentration of the guest is particularly preferably 10% by mass or less to prevent concentration quenching.

The guest may be uniformly contained or may have a concentration gradient in the entire layer in which the host serves as a matrix. Alternatively, the guest may be partially contained in a specific region in the layer, and the light-emitting layer may have a region containing only the host and no guest.

In the present embodiment, the light-emitting layer can contain both an organic compound according to the present embodiment represented by the general formula [1] as a host molecule and a phosphorescent organometallic complex as a guest molecule. The light-emitting layer may further contain a third component. For example, to assist the transfer of an exciton or a carrier, the light-emitting layer may further contain another phosphorescent material in addition to the phosphorescent organometallic complex. Furthermore, to assist the transfer of an exciton or a carrier, the light-emitting layer may further contain a compound different from a compound according to the present embodiment represented by the general formula [1] as a second host.

### <Phosphorescent Organometallic Complex (Guest Molecule)>

The phosphorescent organometallic complex can be an organometallic complex represented by the following general formula [4]:

M(L)m(L′)n(L″)p [4]

### [M]

In the formula [4], M is selected from iridium and platinum. M can be iridium.

### [L, L′, and L″ ]

L, L′, and L″ denote different bidentate ligands.

[m, n, p]

m is selected from an integer of 1 or more and 3 or less, and n and p are independently selected from an integer of 0 or more and 2 or less, provided that m+n+p=3. When m is 2 or more, Ls may be the same or different. When n is 2 or more, L's may be the same or different. When p is 2 or more, L″s may be the same or different.

### [Partial Structure M(L)m]

The partial structure M(L)m is represented by the following general formula [4-1]:

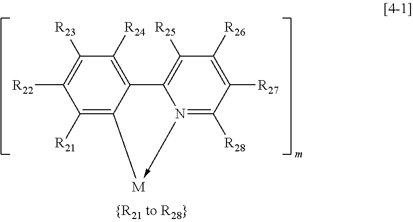

In the formula [4-1], R₂₁ to R₂₈ are independently selected from a hydrogen atom, a deuterium atom, halogen atoms, substituted or unsubstituted alkyl groups, substituted or unsubstituted alkoxy groups, substituted or unsubstituted silyl groups, substituted or unsubstituted aryl groups, substituted or unsubstituted heterocyclic groups, substituted or unsubstituted amino groups, substituted or unsubstituted aryloxy groups, substituted or unsubstituted heteroaryloxy groups, and a cyano group. At least one of R₂₁ to R₂₈ is selected from substituted or unsubstituted aryl groups and substituted or unsubstituted heterocyclic groups.

Examples of the halogen atoms include, but are not limited to, fluorine, chlorine, bromine, and iodine. In particular, a fluorine atom can be used.

Examples of the alkyl groups include, but are not limited to, a methyl group, an ethyl group, a n-propyl group, an isopropyl group, a n-butyl group, a tert-butyl group, a sec-butyl group, an octyl group, a cyclohexyl group, a 1-adamantyl group, and a 2-adamantyl group.

Examples of the alkoxy groups include, but are not limited to, a methoxy group, an ethoxy group, a propoxy group, a 2-ethyl-octyloxy group, and a benzyloxy group.

Examples of the silyl groups include, but are not limited to, a trimethylsilyl group and a triphenylsilyl group.

Examples of the aryl groups include, but are not limited to, a phenyl group, a naphthyl group, an indenyl group, a biphenyl group, a terphenyl group, a fluorenyl group, a phenanthryl group, a fluoranthenyl group, and a triphenylenyl group.

Examples of the heterocyclic groups include, but are not limited to, a pyridyl group, a pyrimidyl group, a pyrazyl group, a triazolyl group, an oxazolyl group, an oxadiazolyl group, a thiazolyl group, a thiadiazolyl group, a carbazolyl group, an acridinyl group, a phenanthrolyl group, a dibenzofuranyl group, and a dibenzothiophenyl group.

Examples of the amino groups include, but are not limited to, an N-methylamino group, an N-ethylamino group, an N,N-dimethylamino group, an N,N-diethylamino group, an N-methyl-N-ethylamino group, an N-benzylamino group, an N-methyl-N-benzylamino group, an N,N-dibenzylamino group, an anilino group, an N,N-diphenylamino group, an N,N-dinaphthylamino group, an N,N-difluorenylamino group, an N-phenyl-N-tolylamino group, an N,N-ditolylamino group, an N-methyl-N-phenylamino group, an N,N-dianisolylamino group, an N-mesityl-N-phenylamino group, an N,N-dimesitylamino group, an N-phenyl-N-(4-t-butylphenyl)amino group, an N-phenyl-N-(4-trifluoromethylphenyl)amino group, an N-piperidyl group, and a carbazolyl group.

Examples of the aryloxy groups and the heteroaryloxy groups include, but are not limited to, a phenoxy group and a thienyloxy group.

An additional optional substituent of the alkyl groups, alkoxy groups, silyl groups, aryl groups, heterocyclic groups, amino groups, aryloxy groups, and heteroaryloxy groups may be, but is not limited to, a deuterium atom; a halogen atom such as fluorine, chlorine, bromine, or iodine; an alkyl group such as a methyl group, an ethyl group, a n-propyl group, an isopropyl group, a n-butyl group, or a tert-butyl group; an alkoxy group, such as a methoxy group, an ethoxy group, or a propoxy group; an amino group, such as a dimethylamino group, a diethylamino group, a dibenzylamino group, a diphenylamino group, or a ditolylamino group; an aryloxy group, such as a phenoxy group; an aromatic hydrocarbon group, such as a phenyl group or a biphenyl group; a heterocyclic group, such as a pyridyl group or a pyrrolyl group; or a cyano group, a hydroxy group, or a thiol group.

Adjacent R₂₁ to R₂₈, in particular, adjacent R₂₁ to R₂₄ or adjacent R₂₅ to R₂₈ may be bonded together and form a ring. The phrase “adjacent R₂₁ to R₂₈ are bonded together and form a ring” means that a ring formed by bonding R₂₁ and R₂₂, R₂₂ and R₂₃, or R₂₃ and R₂₄ together and a benzene ring to which R₂₁ to R₂₄ are bonded form a fused ring, or that a ring formed by bonding R₂₅ and R₂₆, R₂₆ and R₂₇, or R₂₇ and R₂₈ together and a pyridine ring to which R₂₅ to R₂₈ are bonded form a fused ring. The ring formed by bonding adjacent R₂₁ to R₂₈ together may be an aromatic ring. [Partial Structure M(L′)n]

The partial structure M(L′)n is represented by the following general formula [4-2].

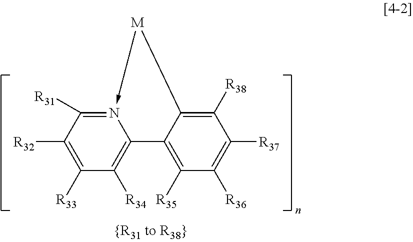

In the formula [4-2], R₃₁ to R₃₈ are independently selected from a hydrogen atom, a deuterium atom, halogen atoms, substituted or unsubstituted alkyl groups, substituted or unsubstituted alkoxy groups, substituted or unsubstituted silyl groups, substituted or unsubstituted aryl groups, substituted or unsubstituted heterocyclic groups, substituted or unsubstituted amino groups, substituted or unsubstituted aryloxy groups, substituted or unsubstituted heteroaryloxy groups, and a cyano group.

Specific examples of the halogen atoms, alkyl groups, alkoxy groups, silyl groups, aryl groups, heterocyclic groups, amino groups, aryloxy groups, and heteroaryloxy groups include, but are not limited to, those described for R₂₁ to R₂₈. Specific examples of an additional optional substituent of the alkyl groups, alkoxy groups, silyl groups, aryl groups, heterocyclic groups, amino groups, aryloxy groups, and heteroaryloxy groups include, but are not limited to, those described for R₂₁ to R₂₈.

Adjacent R₃₁ to R₃₈, in particular, adjacent R₃₁ to R₃₄ or adjacent R₃₅ to R₃₈ may be bonded together and form a ring. The phrase “adjacent R₃₁ to R₃₈ are bonded together and form a ring” means that a ring formed by bonding R₃₁ and R₃₂, R₃₂ and R₃₃, or R₃₃ and R₃₄ together and a pyridine ring to which R₃₁ to R₃₄ are bonded form a fused ring, or that a ring formed by bonding R₃₅ and R₃₆, R₃₆ and R₃₇, or R₃₇ and R₃₈ together and a benzene ring to which R₃₅ to R₃₈ are bonded form a fused ring. The ring formed by bonding adjacent R₃₁ to R₃₈ together may be an aromatic ring.

### [Partial Structure M(L″)p]

The partial structure M(L″)p is represented by the following general formula [4-3].

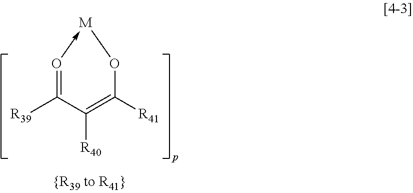

In the formula [4-3], R₃₉ to R₄₁ are independently selected from a hydrogen atom, a deuterium atom, halogen atoms, substituted or unsubstituted alkyl groups, substituted or unsubstituted alkoxy groups, substituted or unsubstituted silyl groups, substituted or unsubstituted aryl groups, substituted or unsubstituted heterocyclic groups, substituted or unsubstituted amino groups, substituted or unsubstituted aryloxy groups, substituted or unsubstituted heteroaryloxy groups, and a cyano group.

Specific examples of the halogen atoms, alkyl groups, alkoxy groups, silyl groups, aryl groups, heterocyclic groups, amino groups, aryloxy groups, and heteroaryloxy groups include, but are not limited to, those described for R₂₁ to R₂₈. Specific examples of an additional optional substituent of the alkyl groups, alkoxy groups, silyl groups, aryl groups, heterocyclic groups, amino groups, aryloxy groups, and heteroaryloxy groups include, but are not limited to, those described for R₂₁ to R₂₈.

In the organometallic complexes represented by the general formula [4], the partial structure M(L)m can have a tricyclic or higher cyclic fused ring. This is because the tricyclic or higher cyclic fused-ring skeleton improve planarity, promotes energy transfer from a host molecule, and improves efficiency and durability. Examples of the tricyclic or higher cyclic fused ring include a phenanthrene ring, a triphenylene ring, a benzofluorene ring, a dibenzofuran ring, a dibenzothiophene ring, a benzonaphthofuran ring, a benzonaphthothiophene ring, a benzoisoquinoline ring, and a naphthoisoquinoline ring.

### SPECIFIC EXAMPLES

Specific examples of the partial structure M(L)m of an organometallic complex serving as a guest include, but are not limited to, those described below. In these specific examples, a coordinate bond is indicated by a straight line, a dotted line, or an arrow.

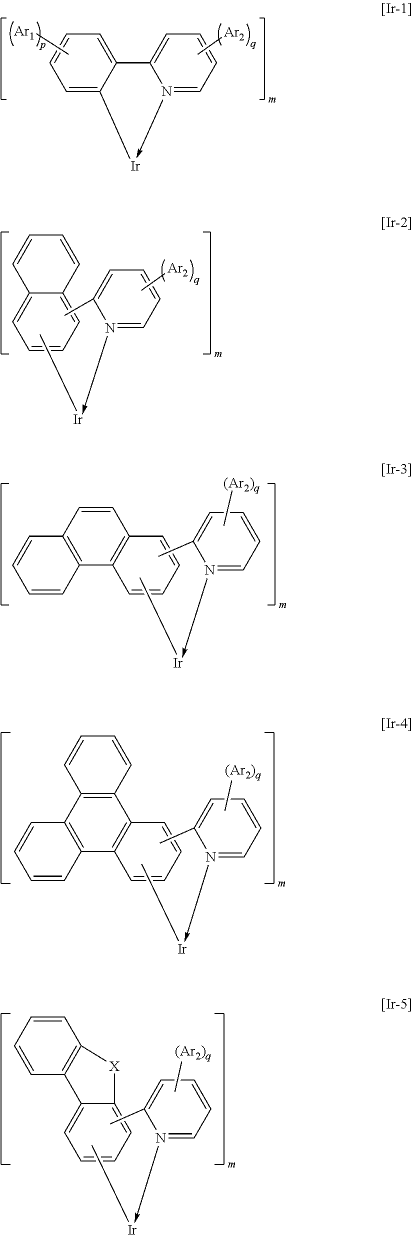

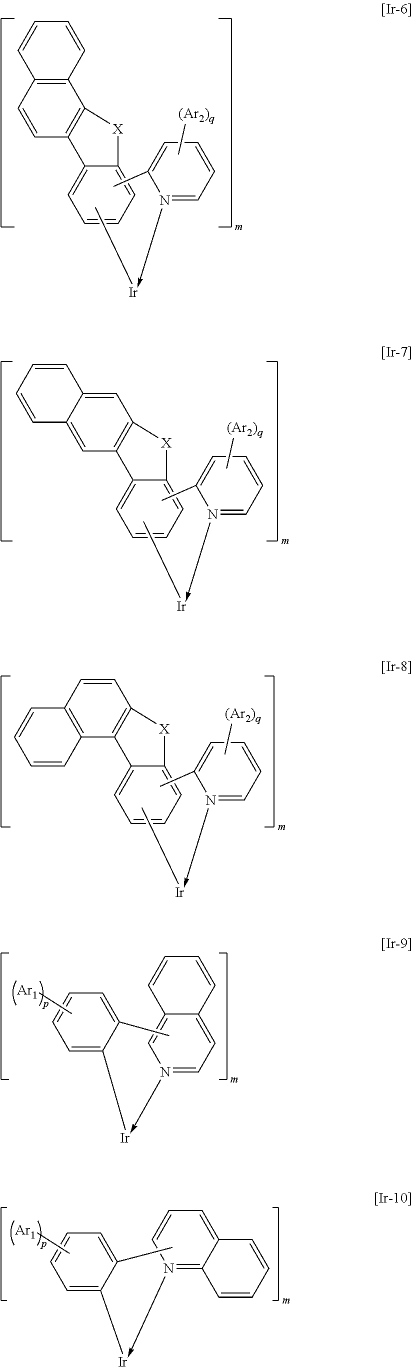

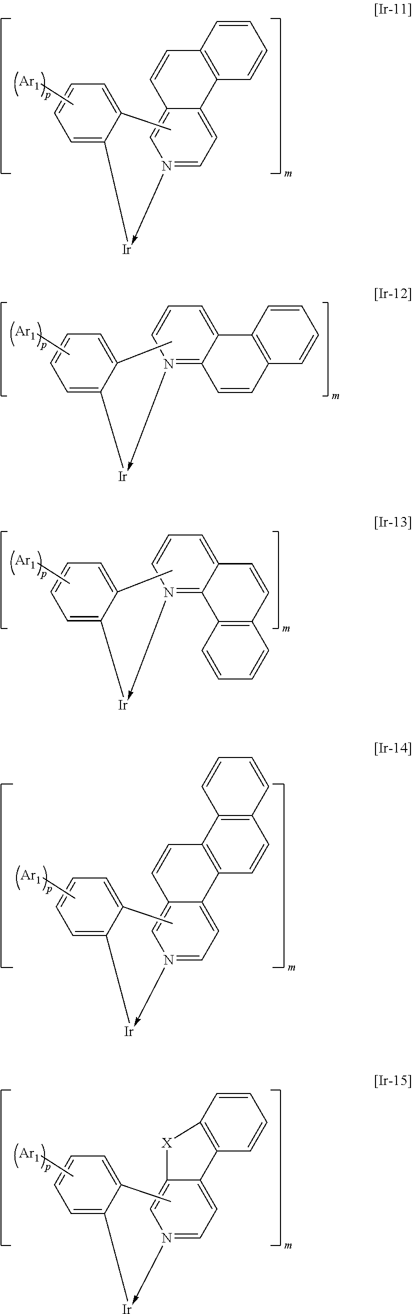

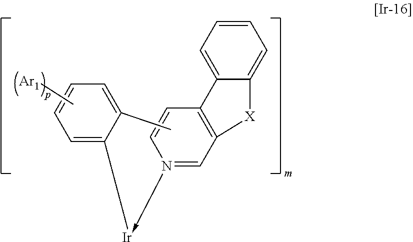

In the general formulae [Ir-5] to [Ir-8] and [Ir-15] to [Ir-16], X′ is selected from an oxygen atom, a sulfur atom, substituted or unsubstituted carbon atoms, and substituted or unsubstituted nitrogen atoms.

In the general formulae [Ir-2] to [Ir-8], adjacent R₂₁ to R₂₄ are bonded together and form a ring. In the general formulae [Ir-9] to [Ir-16], adjacent R₂₅ to R₂₈ are bonded together and form a ring. In the general formulae [Ir-3] to [Ir-8], at least one of R₂₁ to R₂₄ is a phenyl group or a naphthyl group and forms a ring with an adjacent group. In the general formulae [Ir-11] to [Ir-16], at least one of R₂₅ to R₂₈ is a phenyl group or a naphthyl group and forms a ring with an adjacent group. Thus, the general formulae [Ir-3] to [Ir-8] and [Ir-11] to [Ir-16] may or may not further have an aryl group or a heterocyclic group.

A metal complex with the partial structure M(L)m represented by one of the general formulae [Ir-1] to [Ir-16] can have a tricyclic or higher cyclic fused ring as a ligand. More specifically, a metal complex can have the partial structure M(L)m represented by one of the general formulae [Ir-3] to [Ir-8] and [Ir-11] to [Ir-16]. Specific examples of such a metal complex include, but are not limited to, those described below.

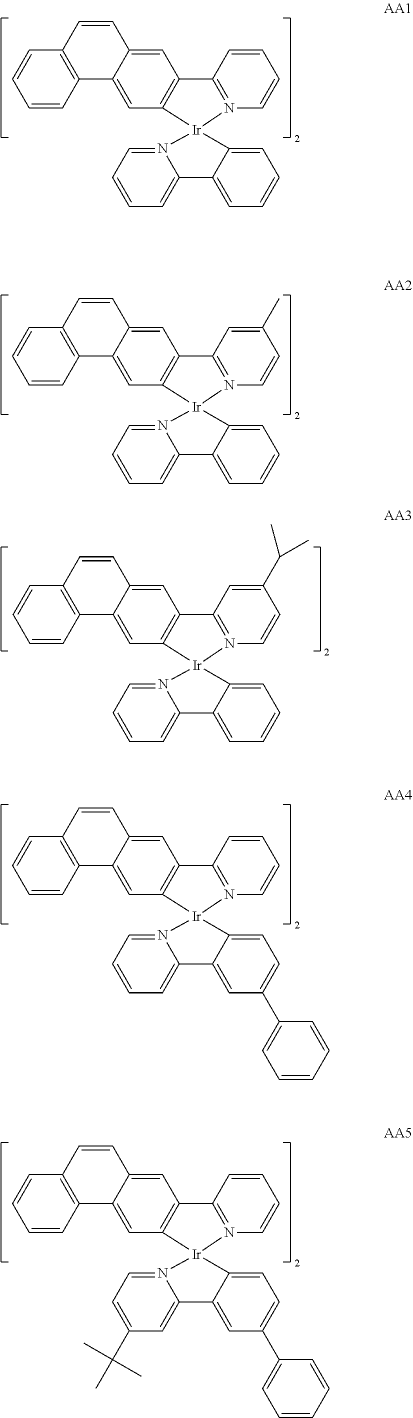

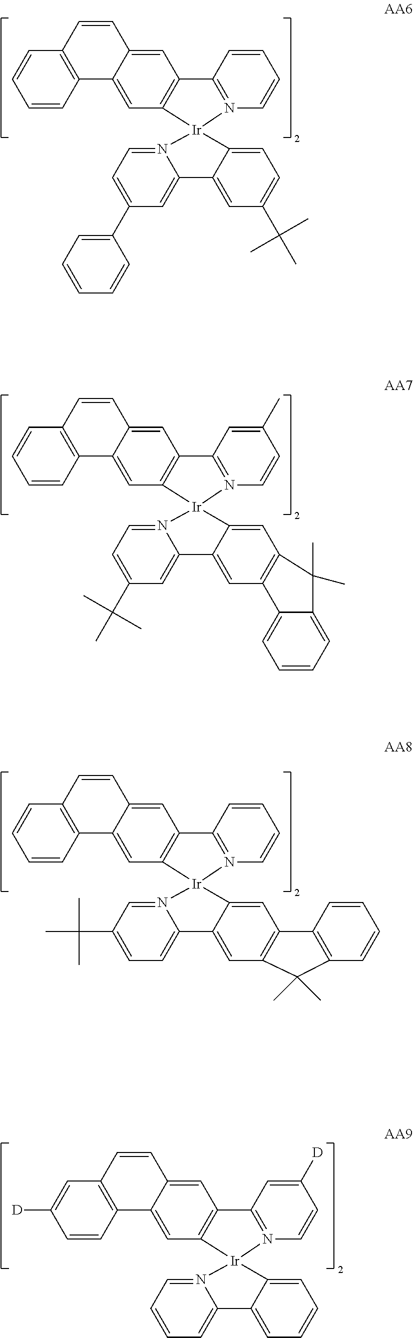

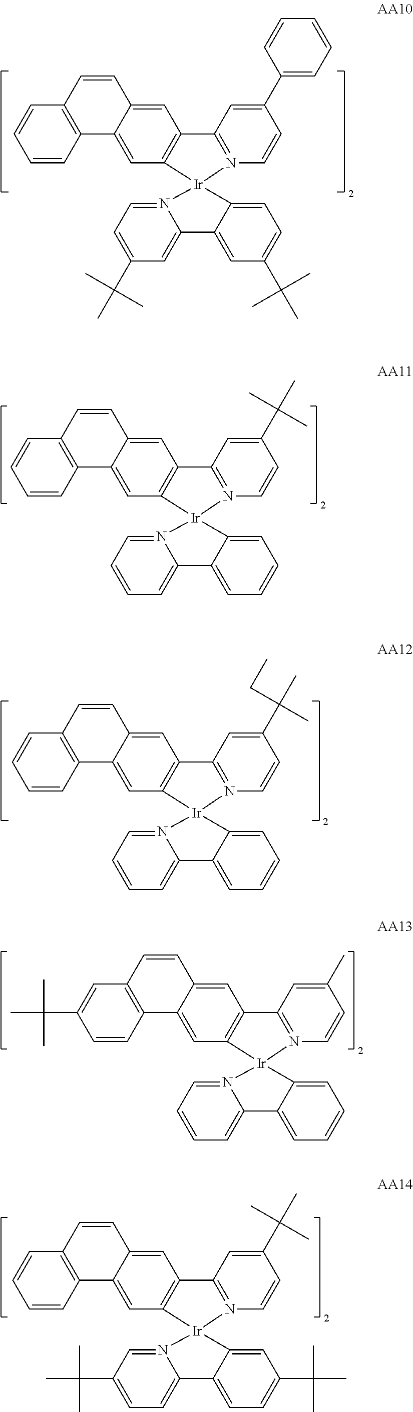

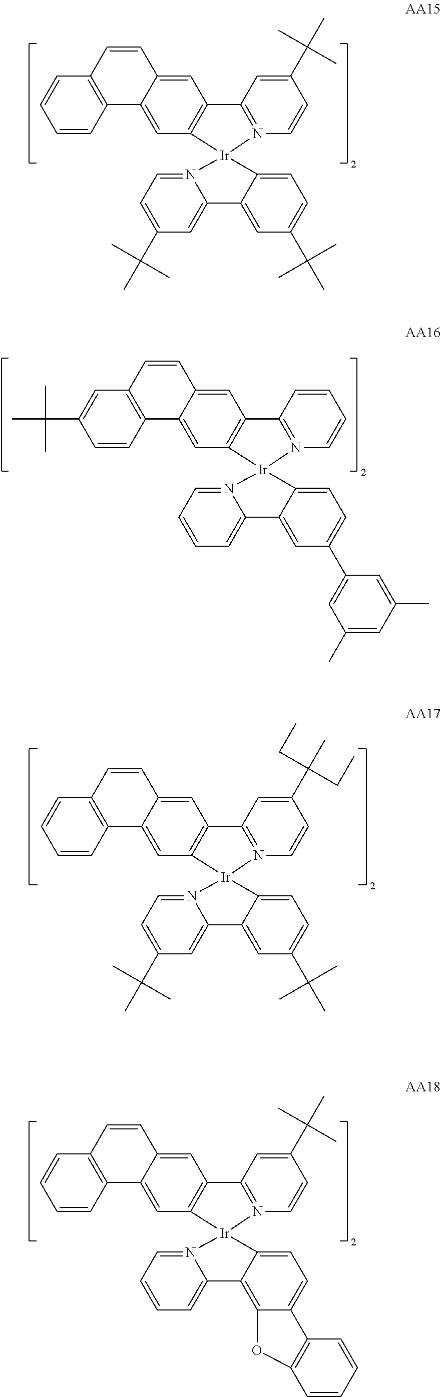

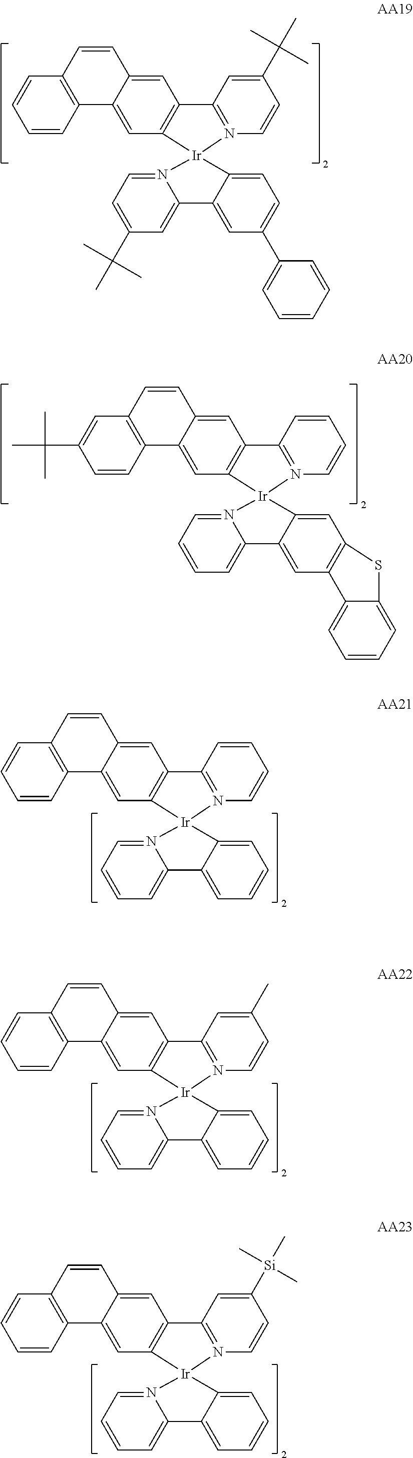

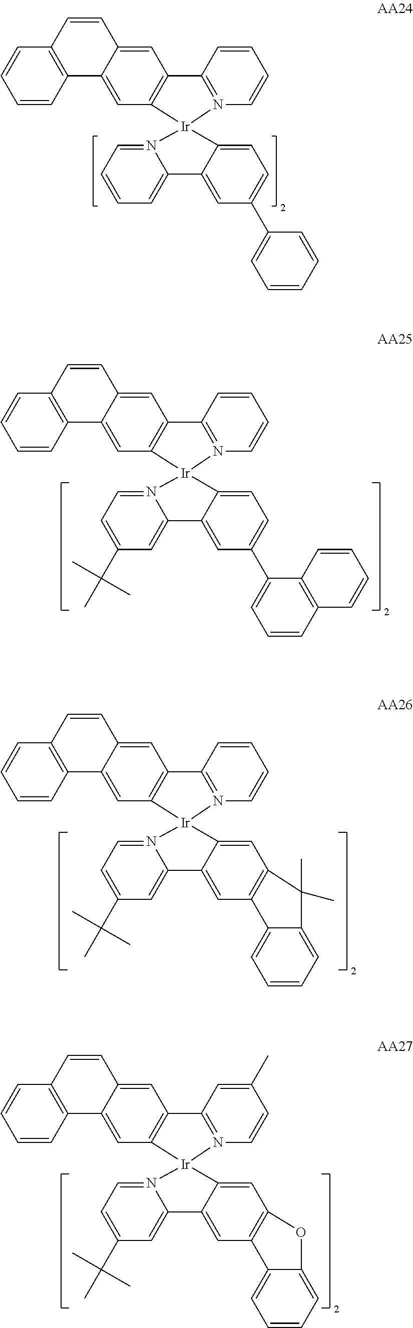

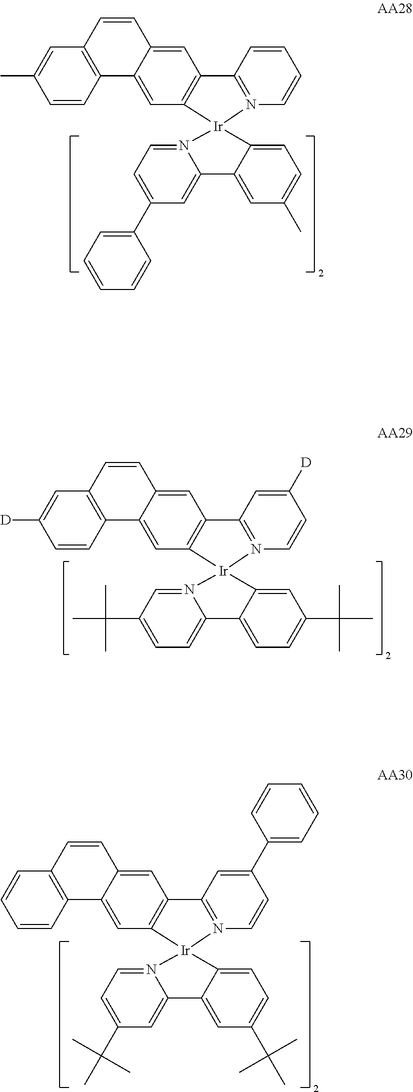

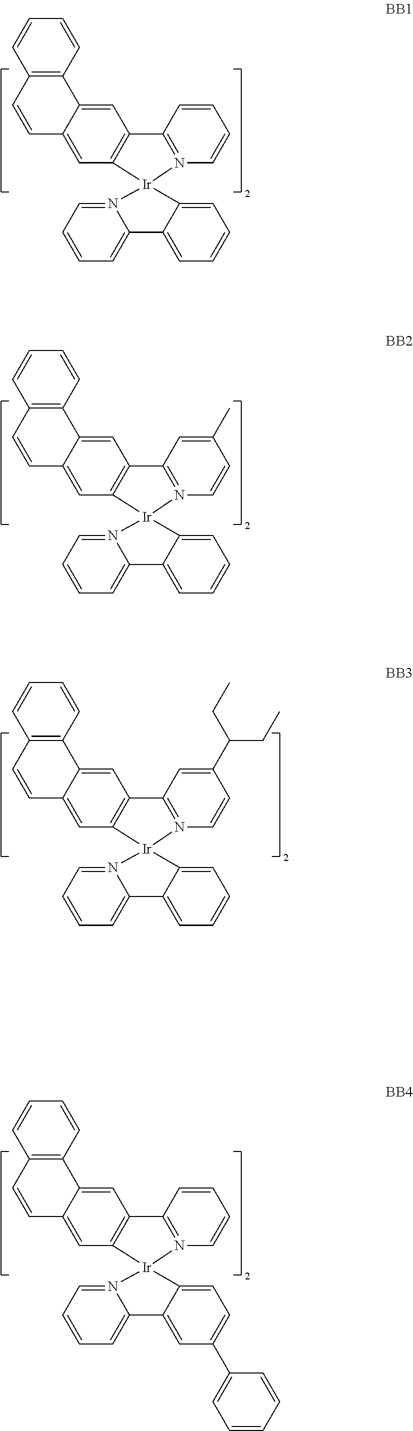

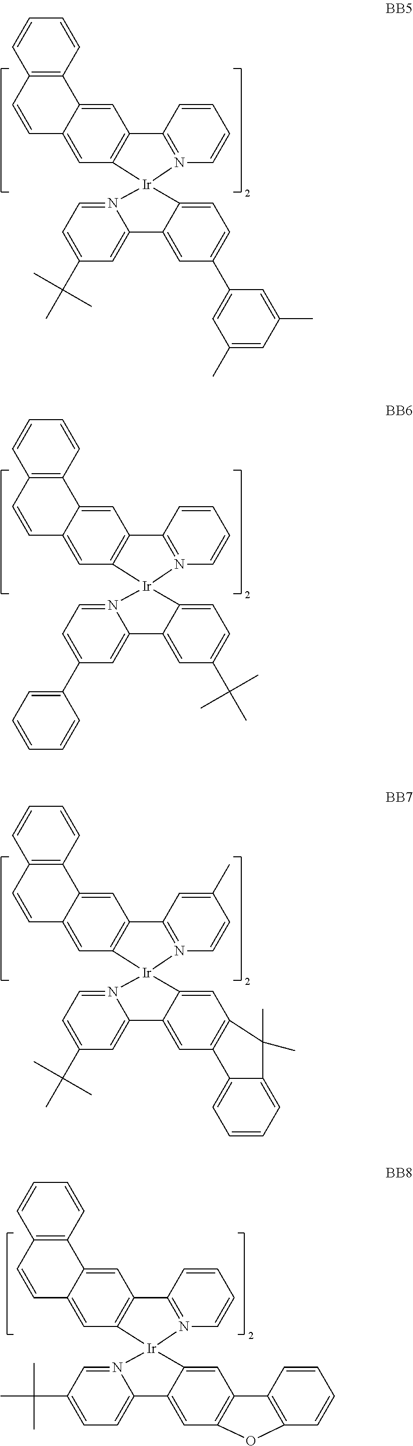

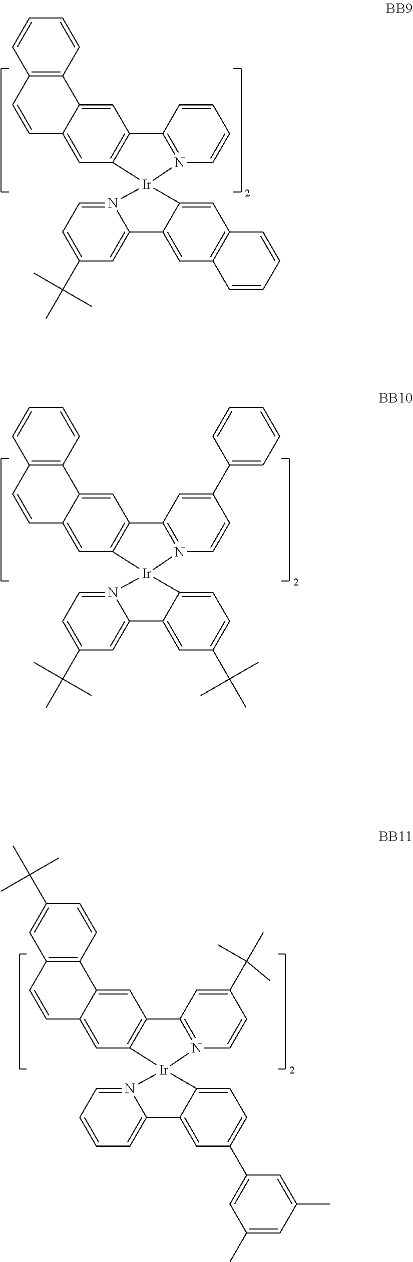

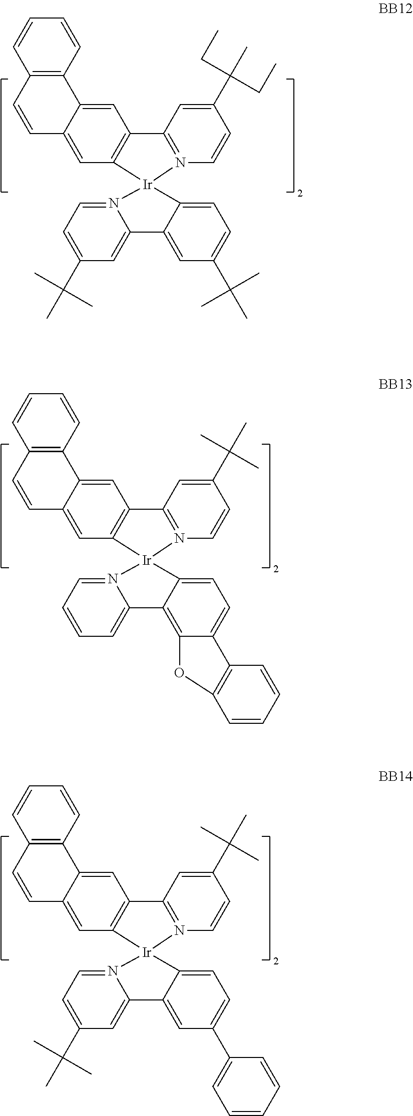

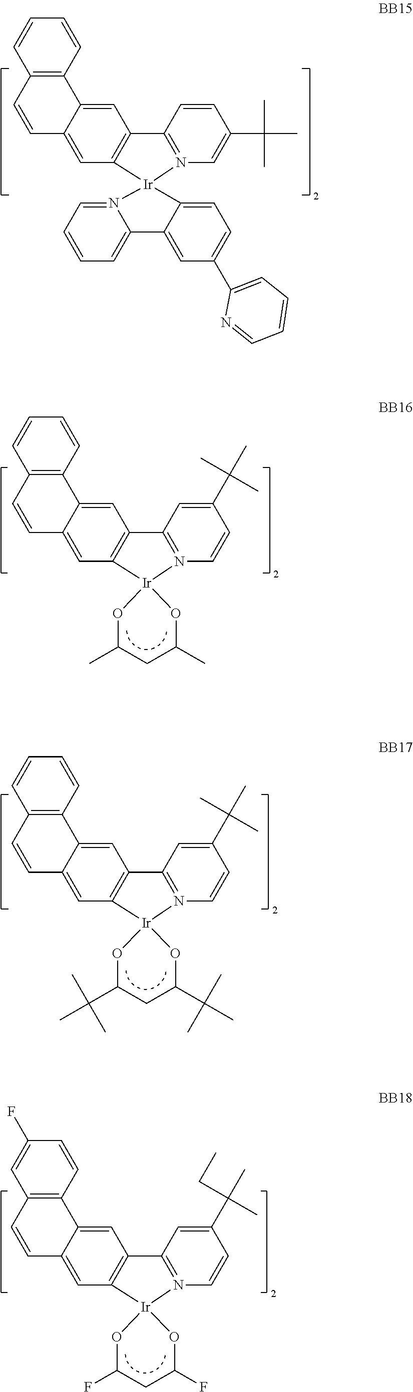

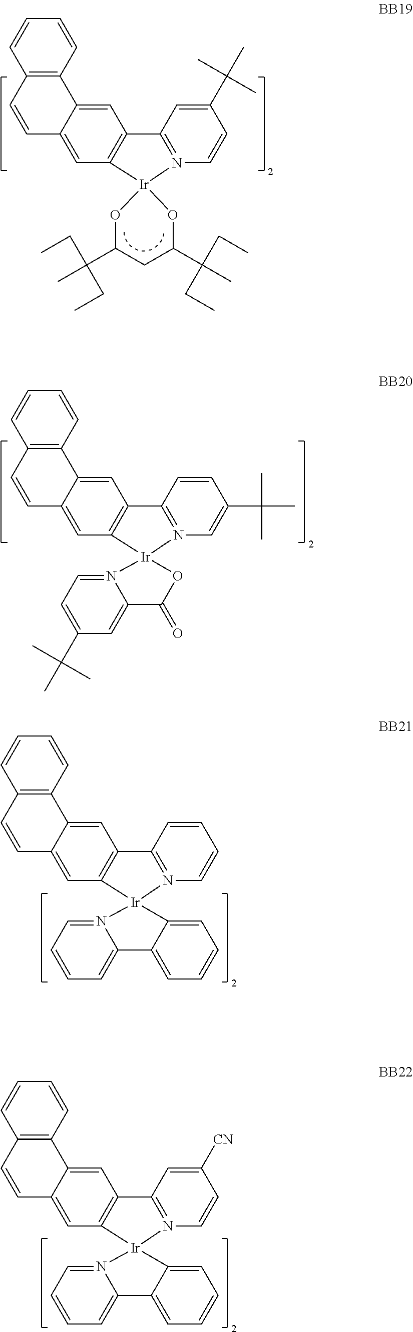

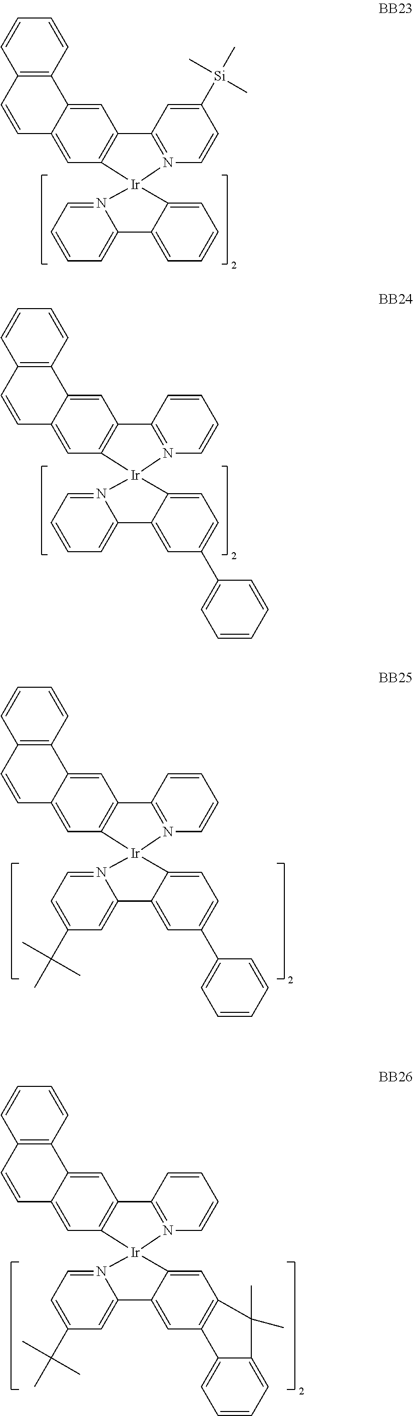

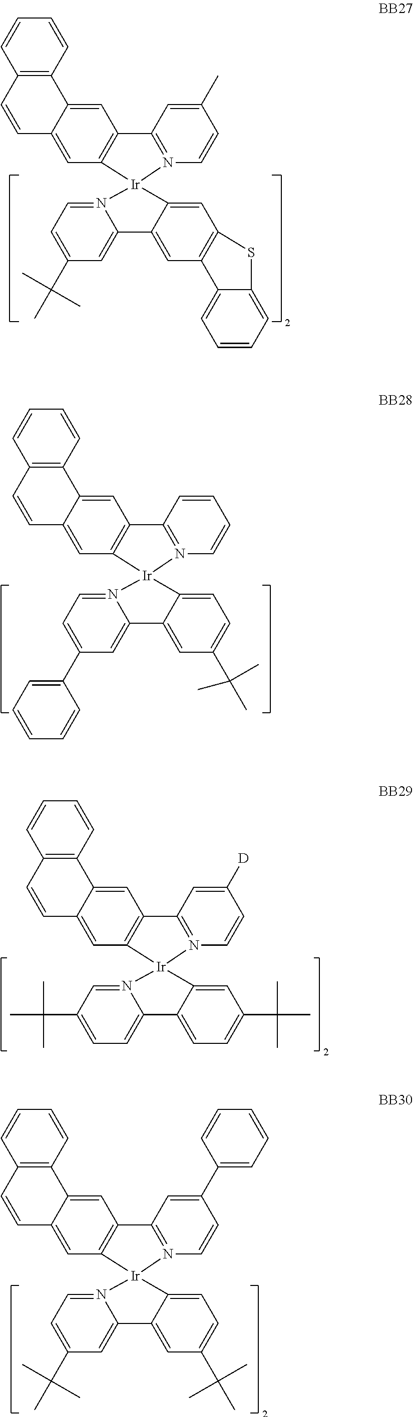

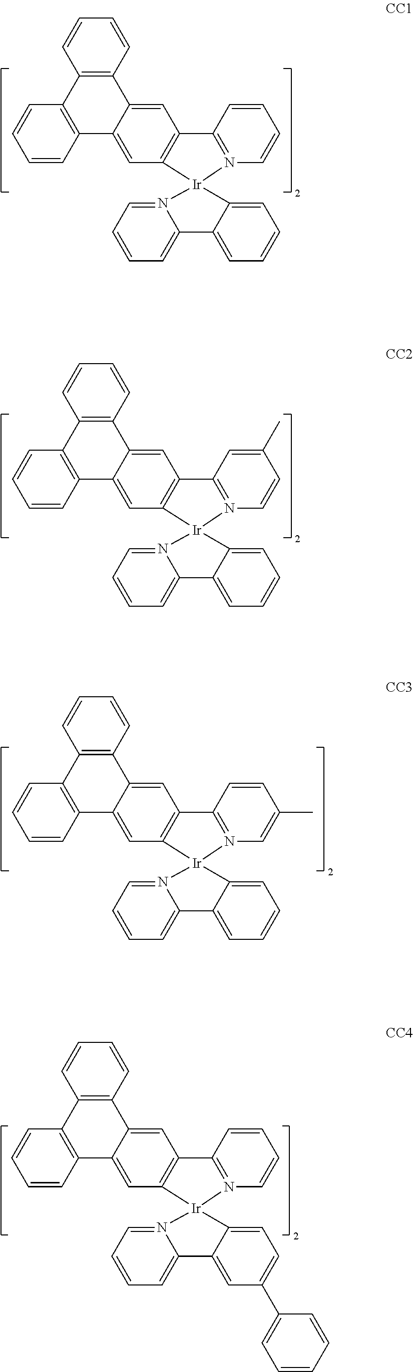

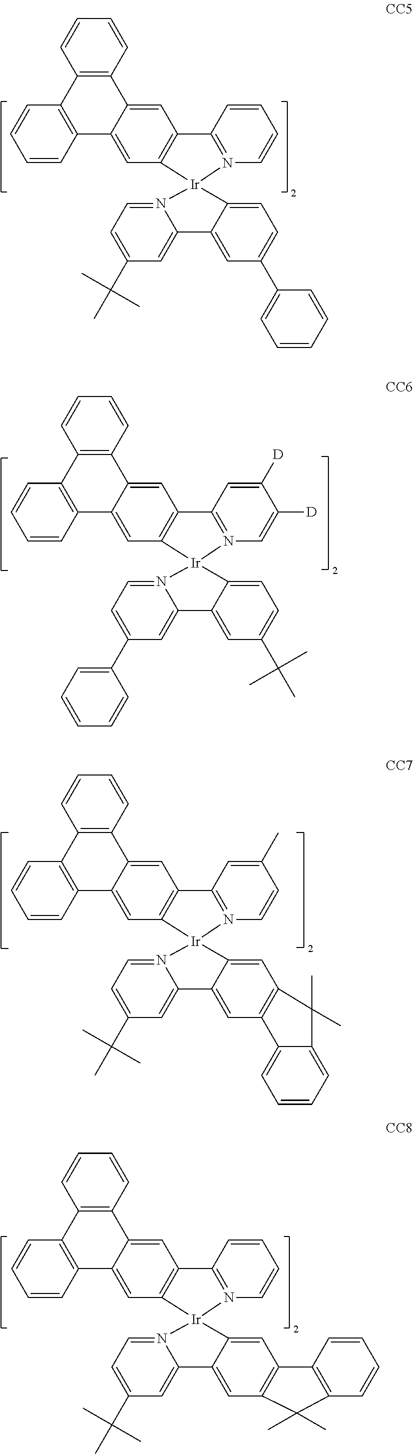

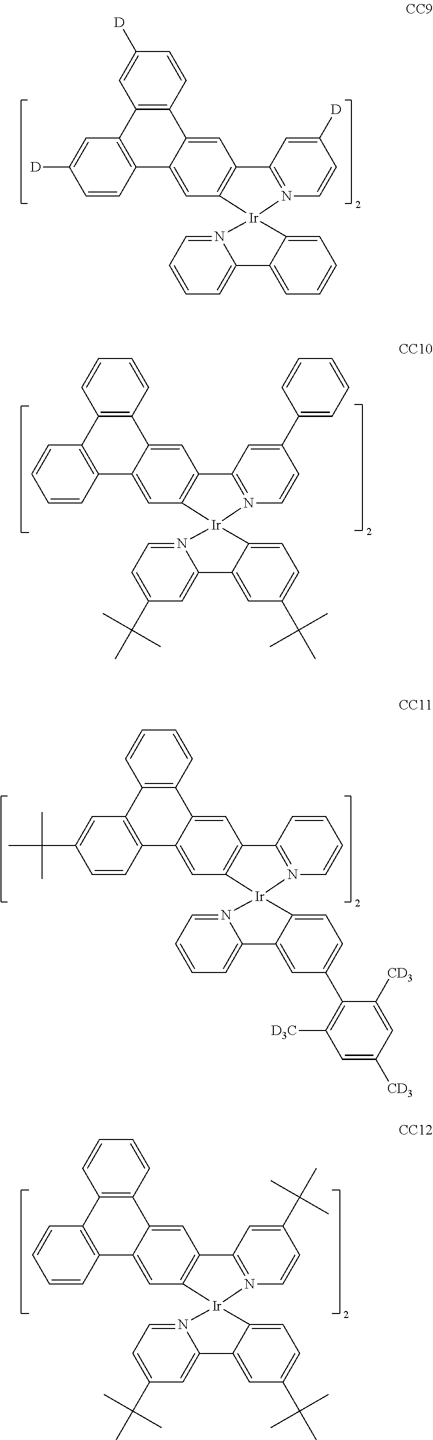

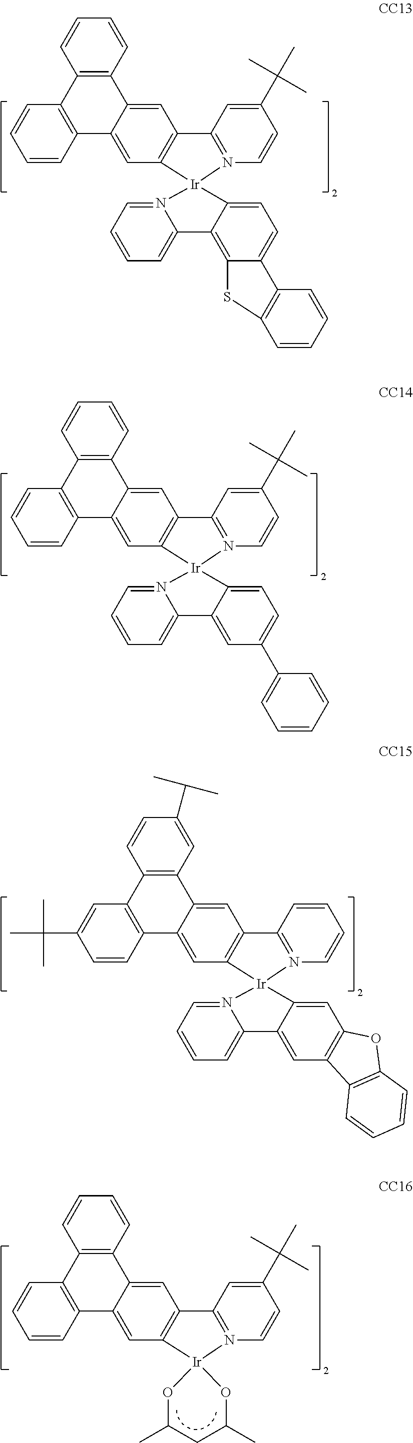

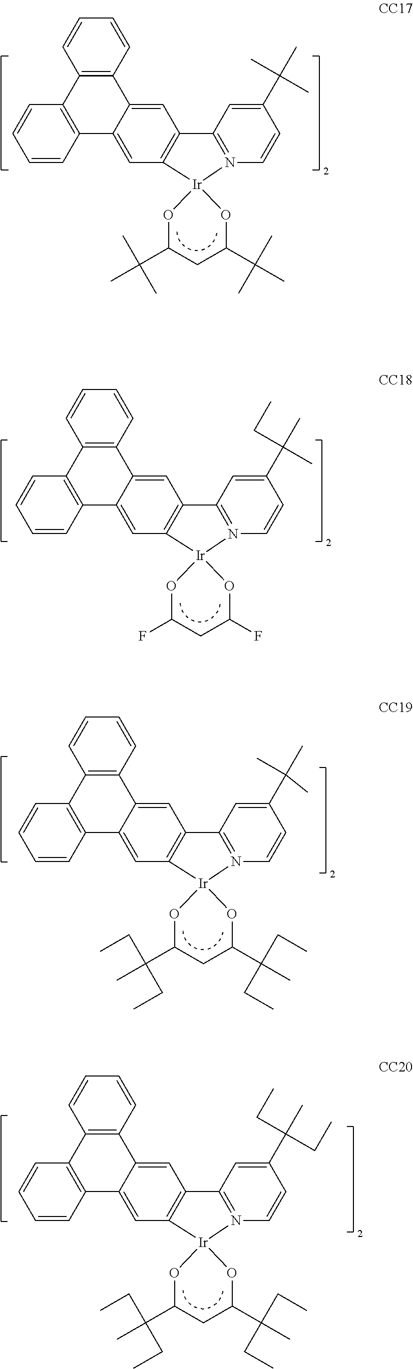

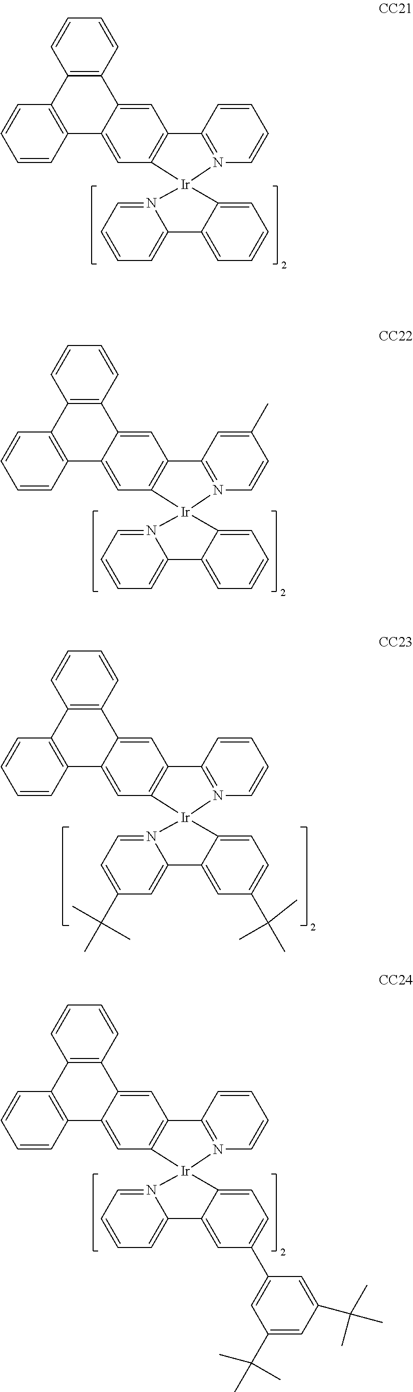

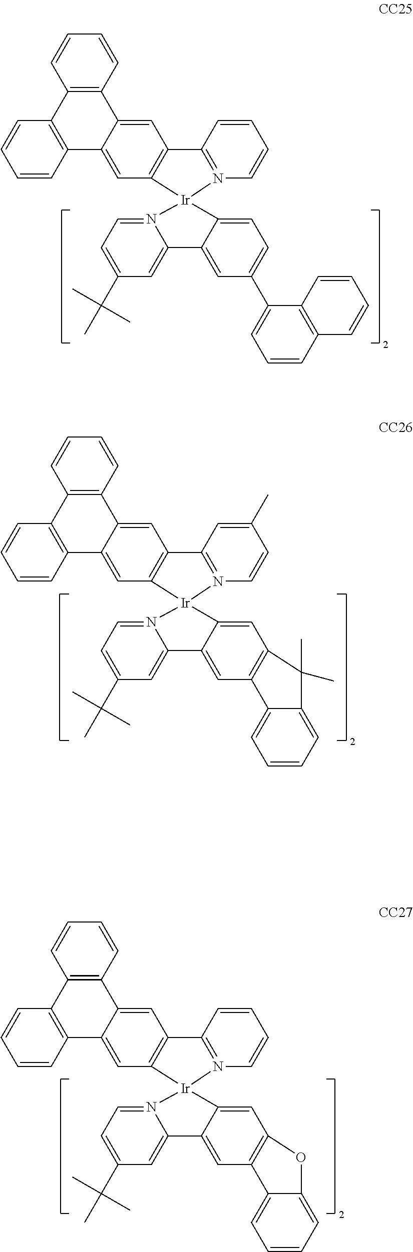

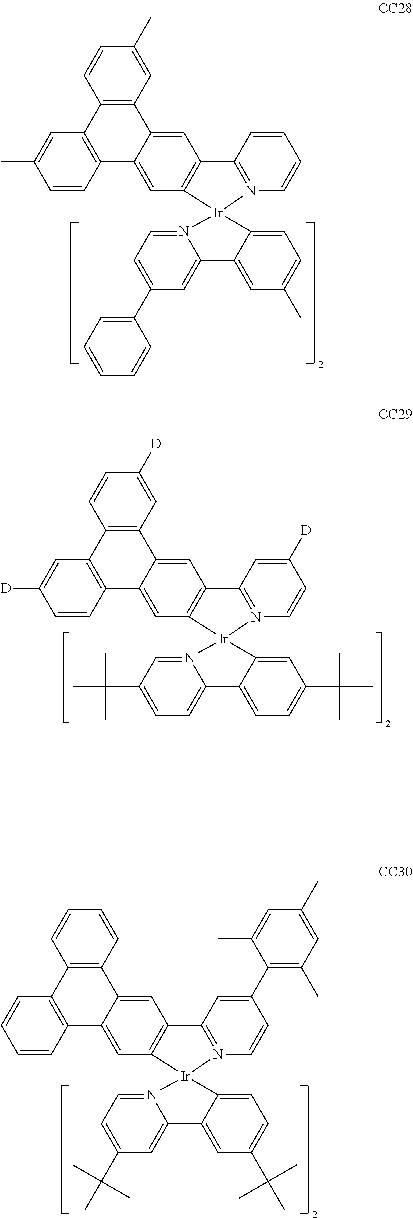

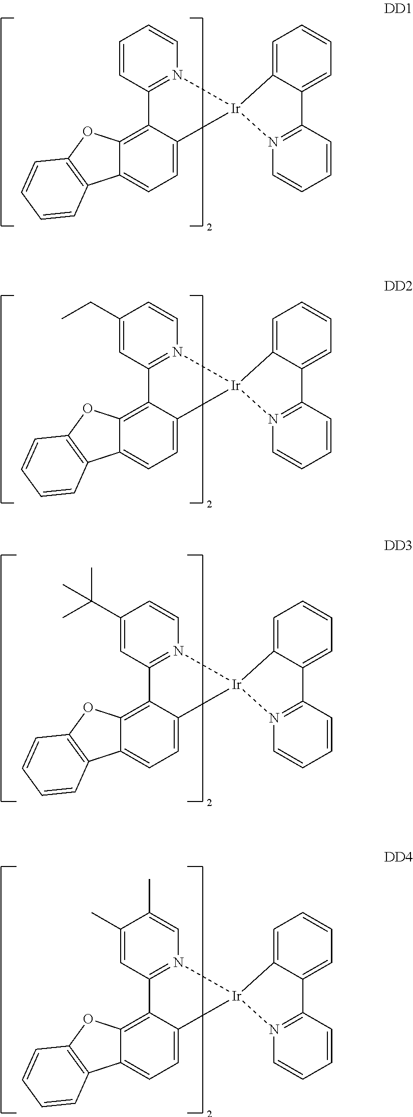

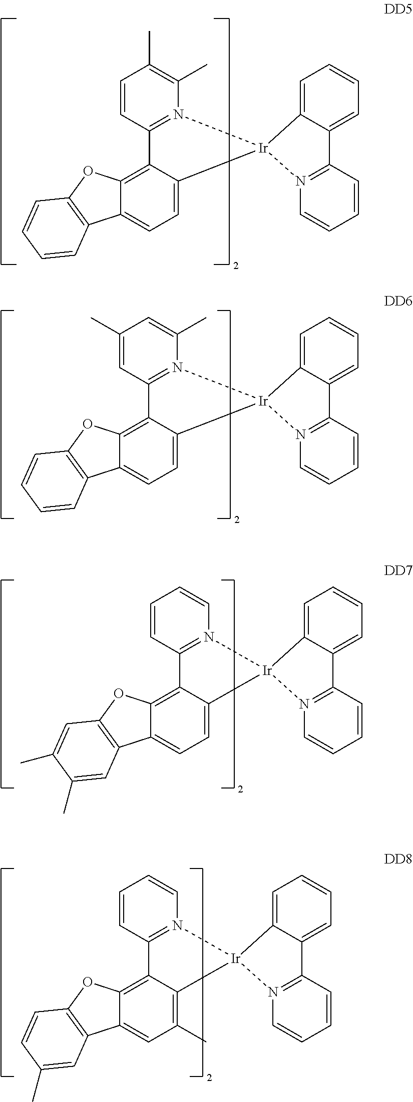

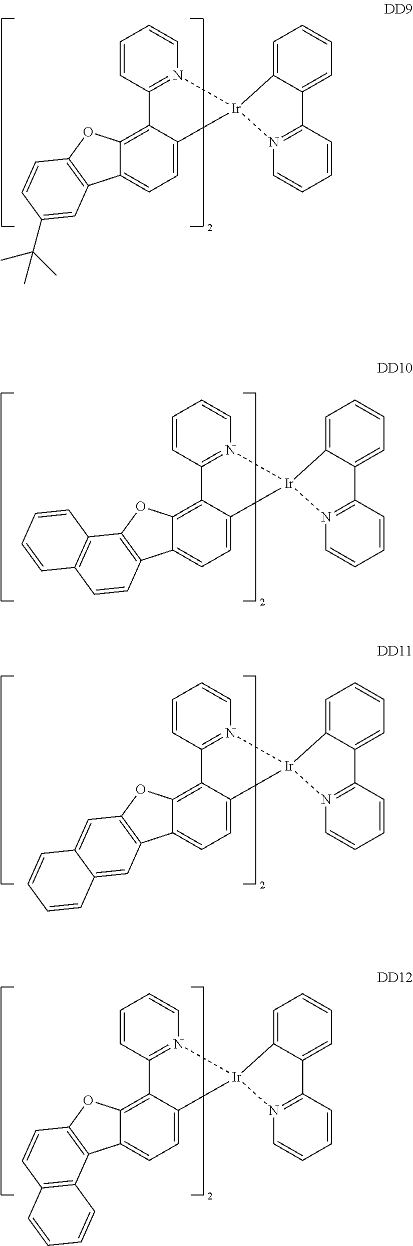

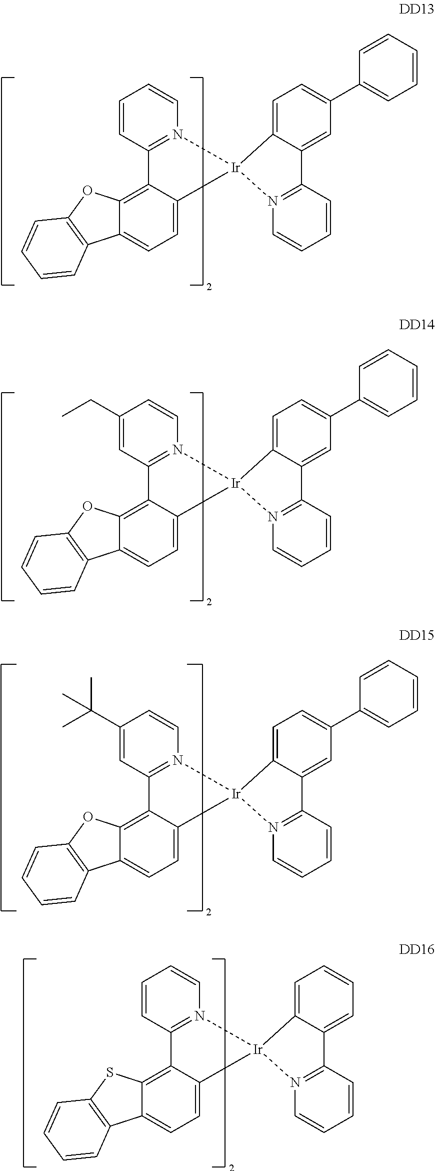

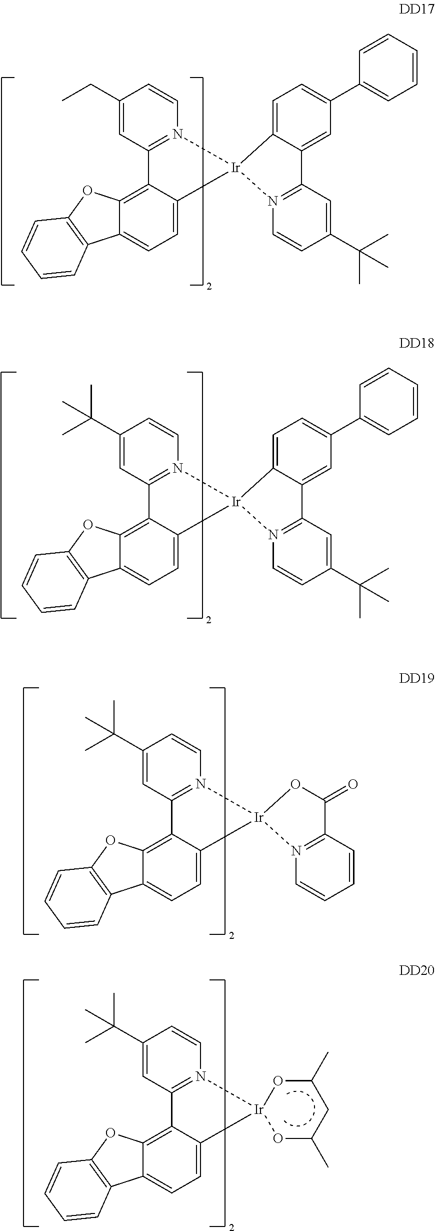

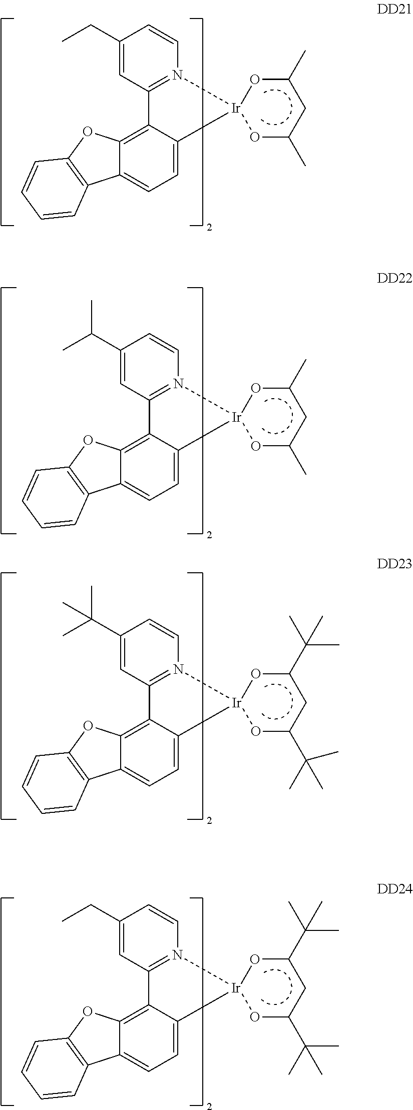

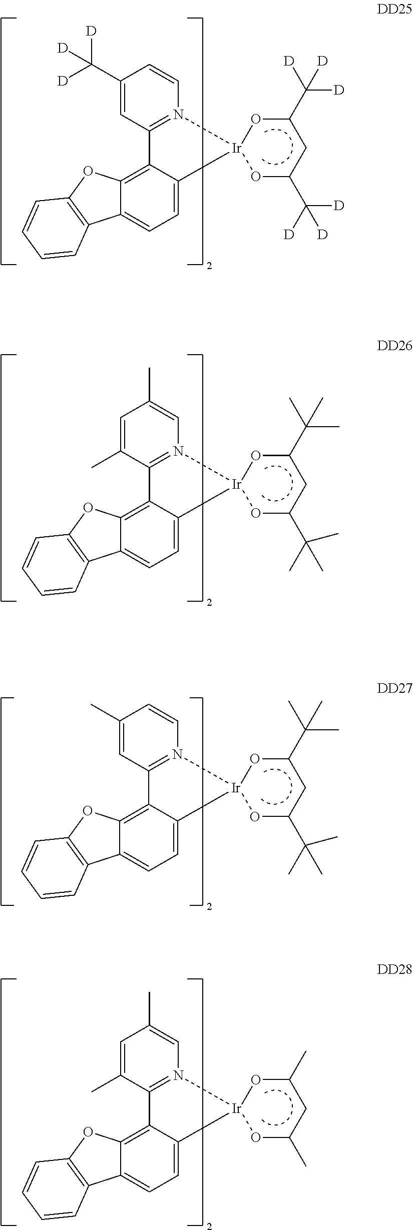

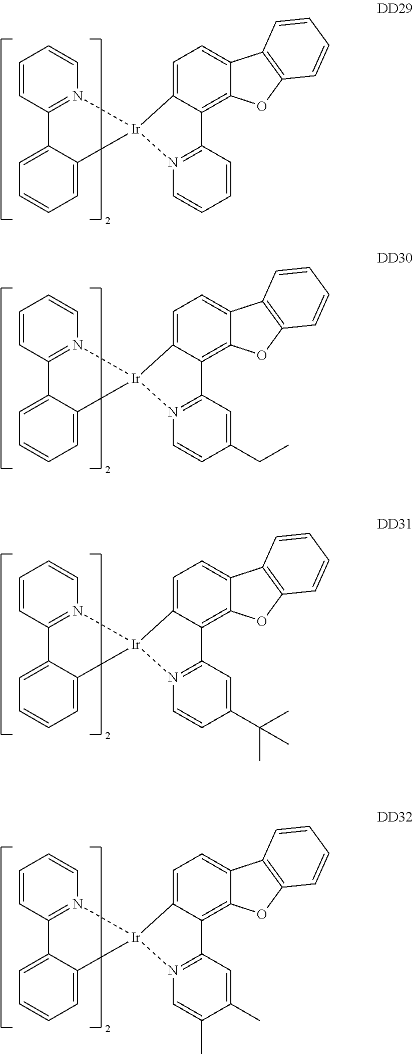

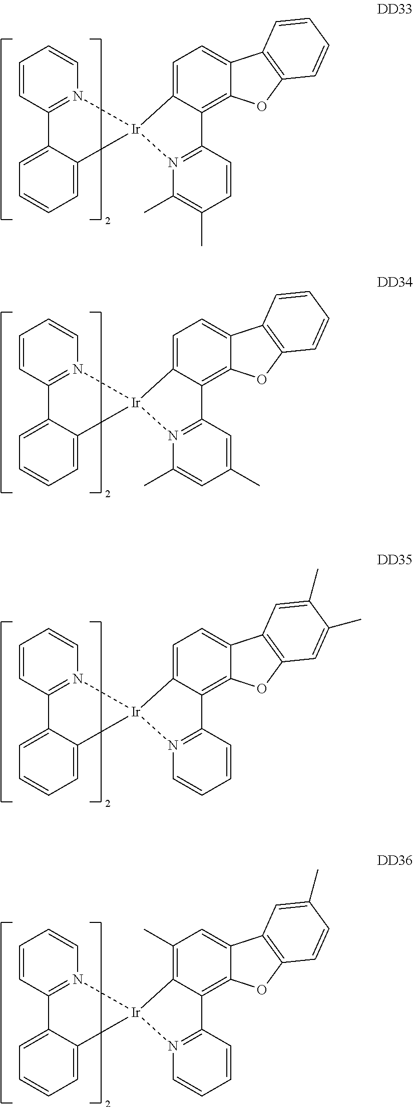

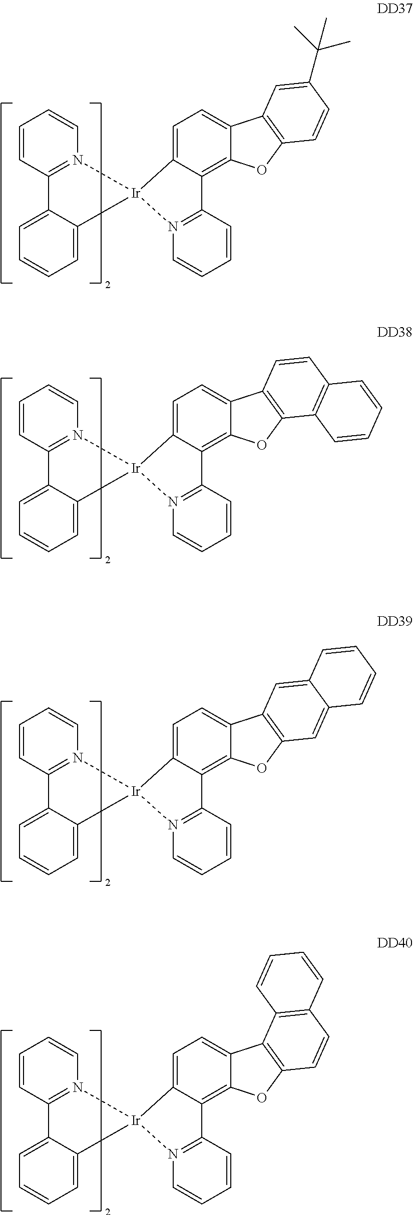

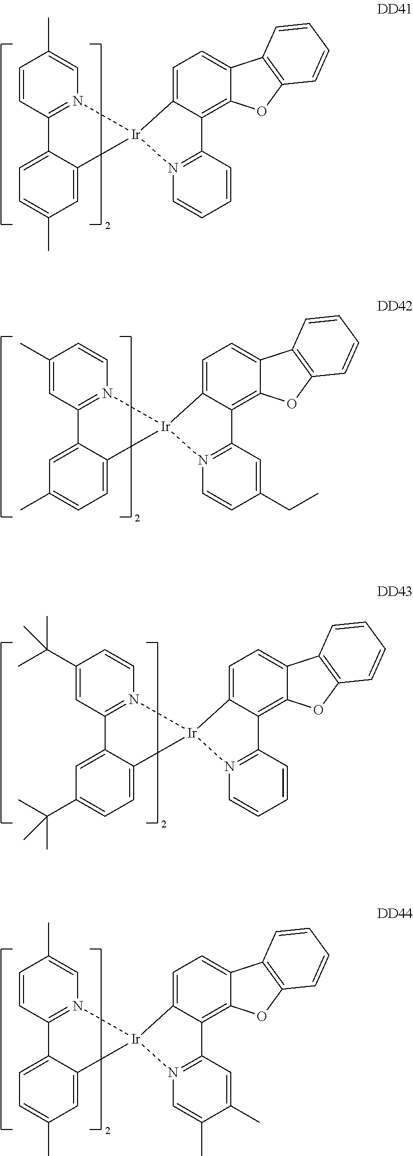

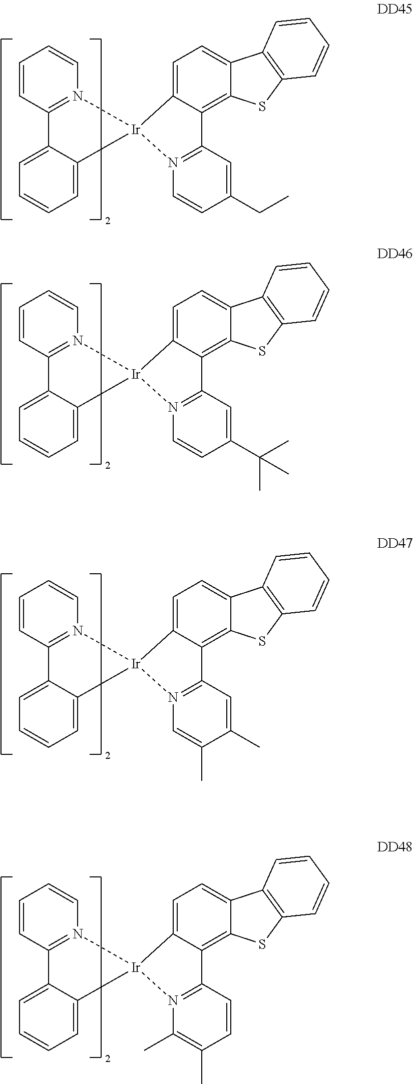

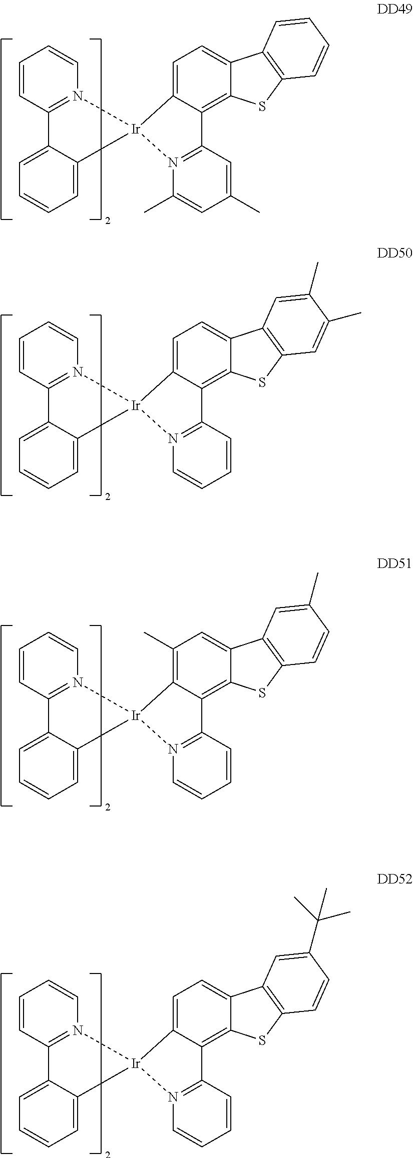

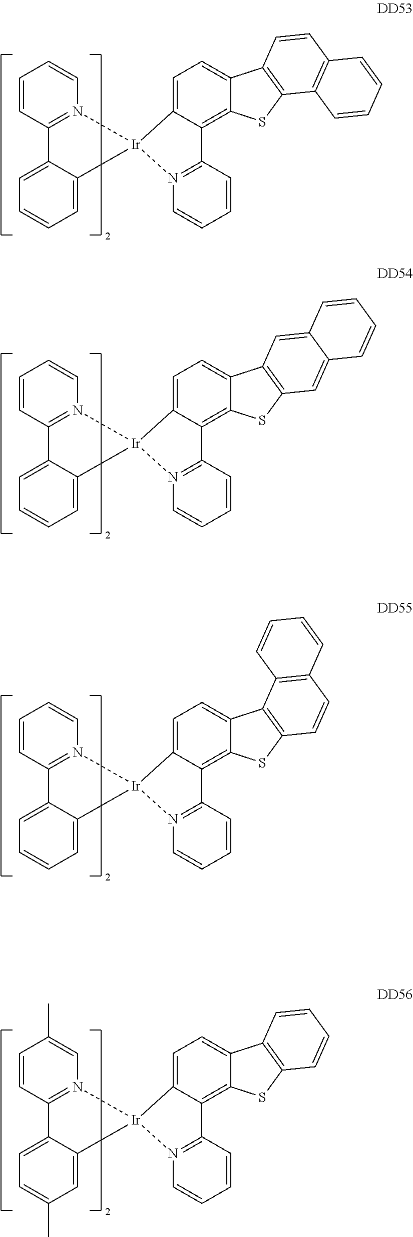

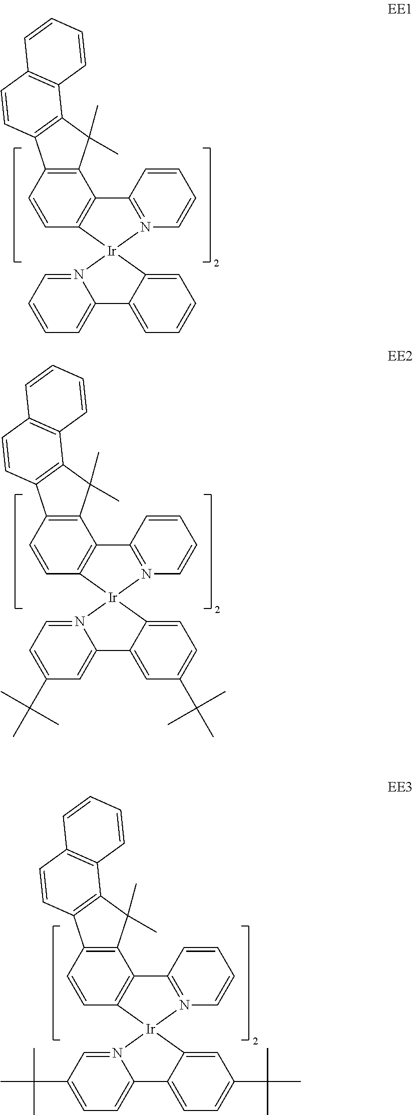

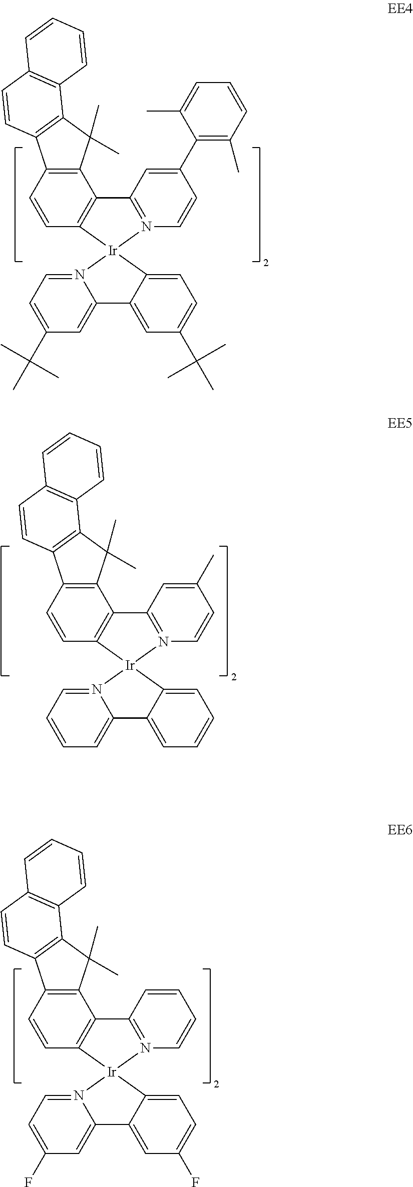

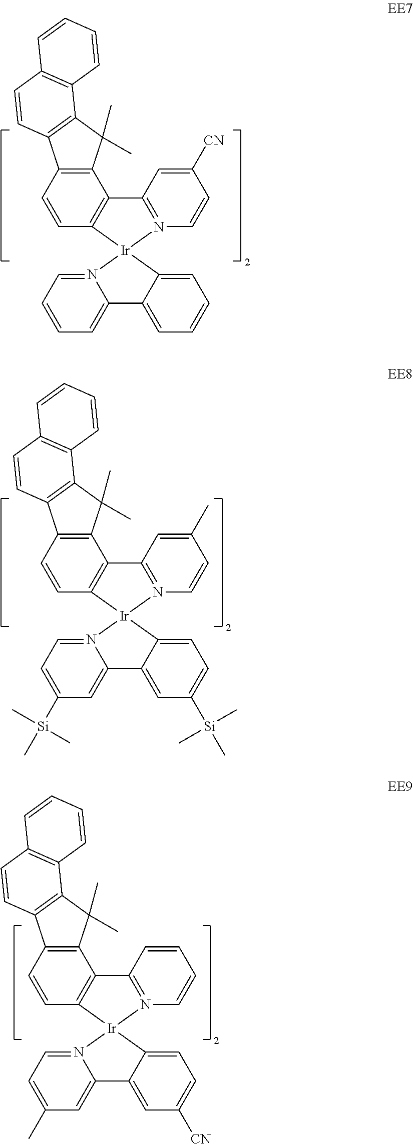

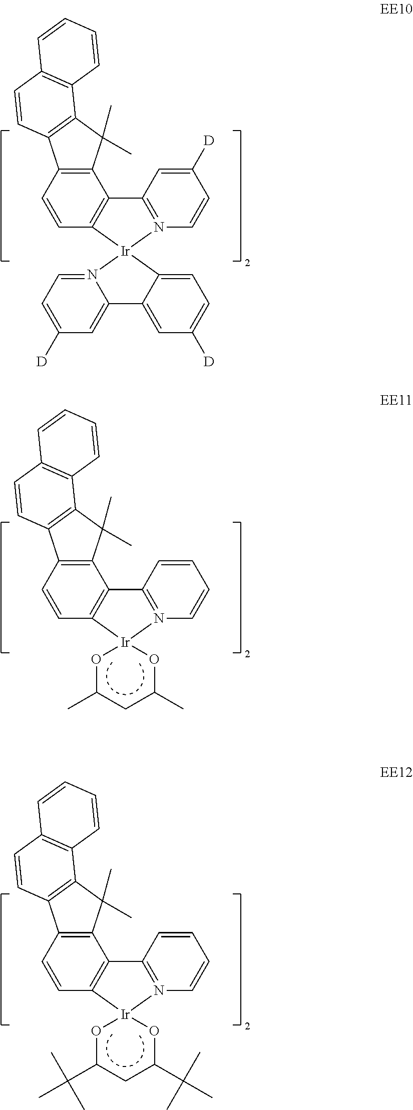

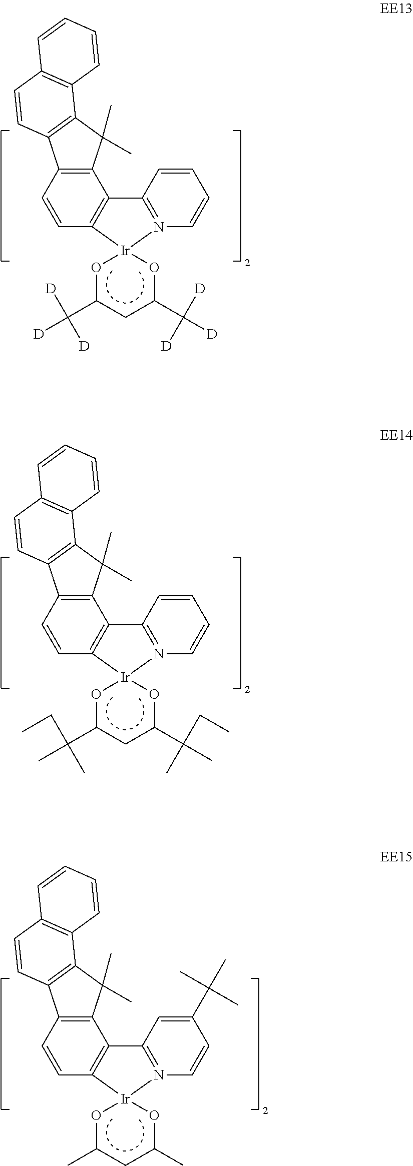

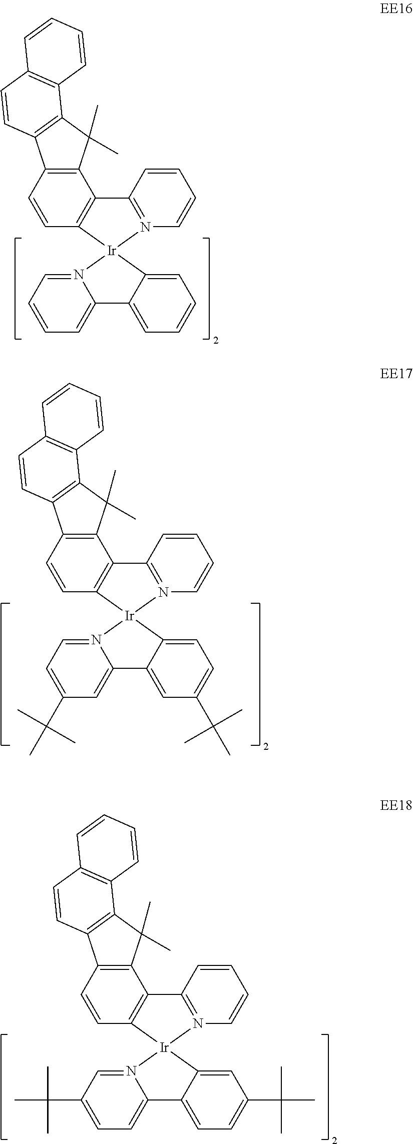

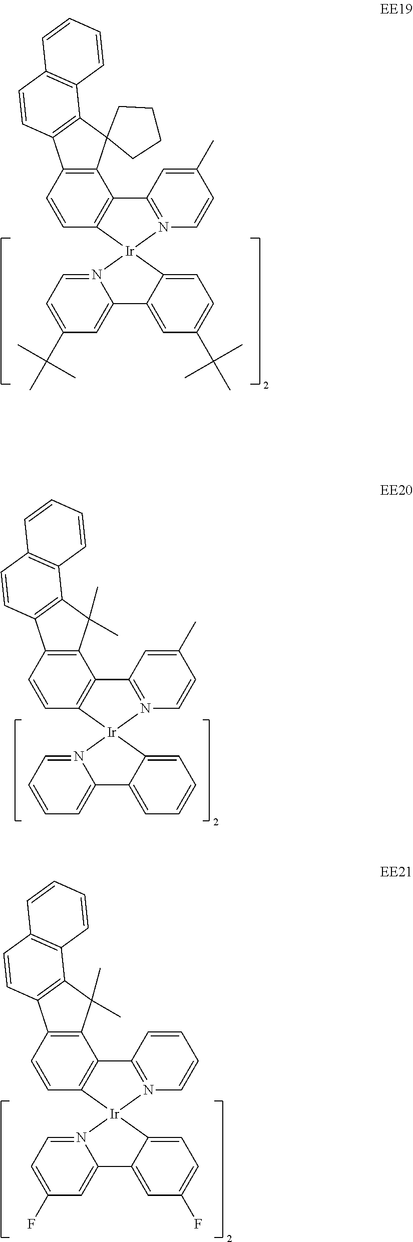

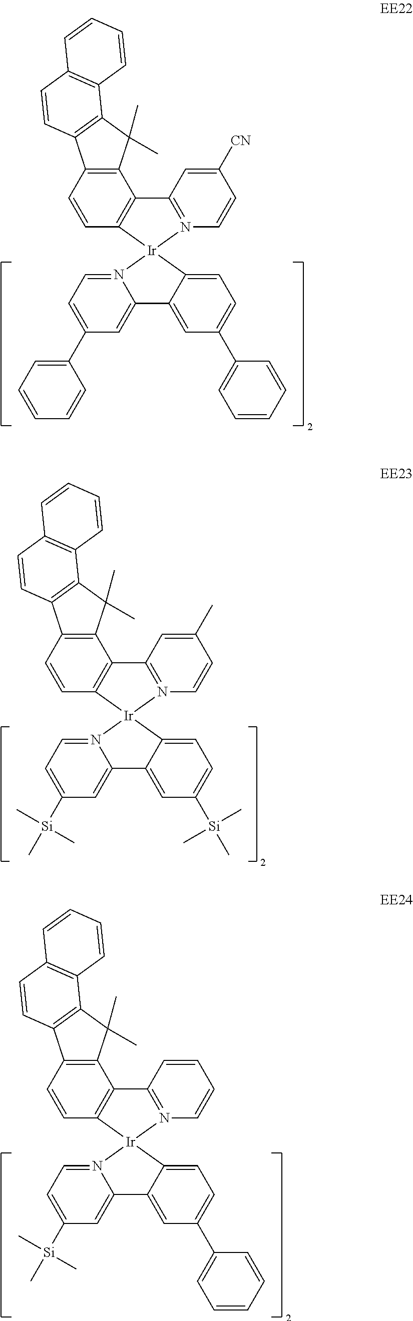

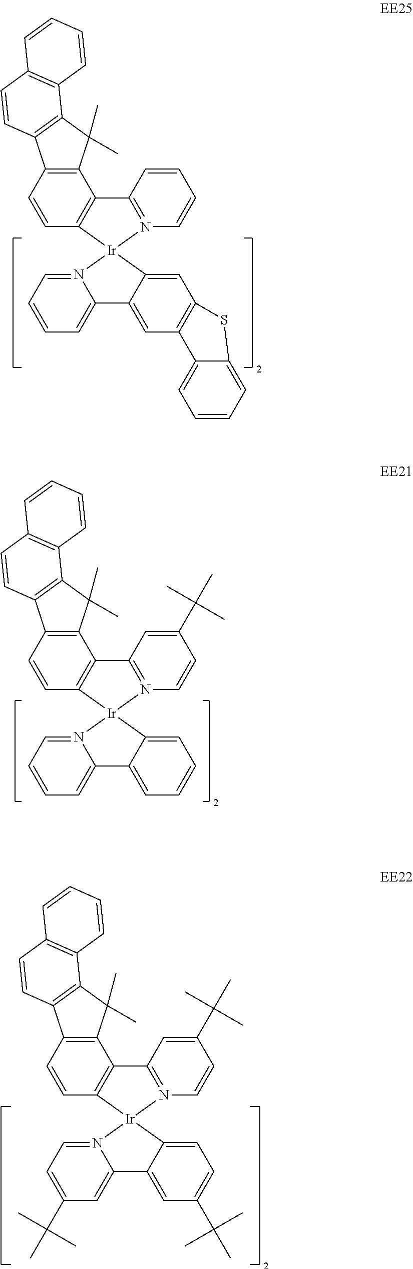

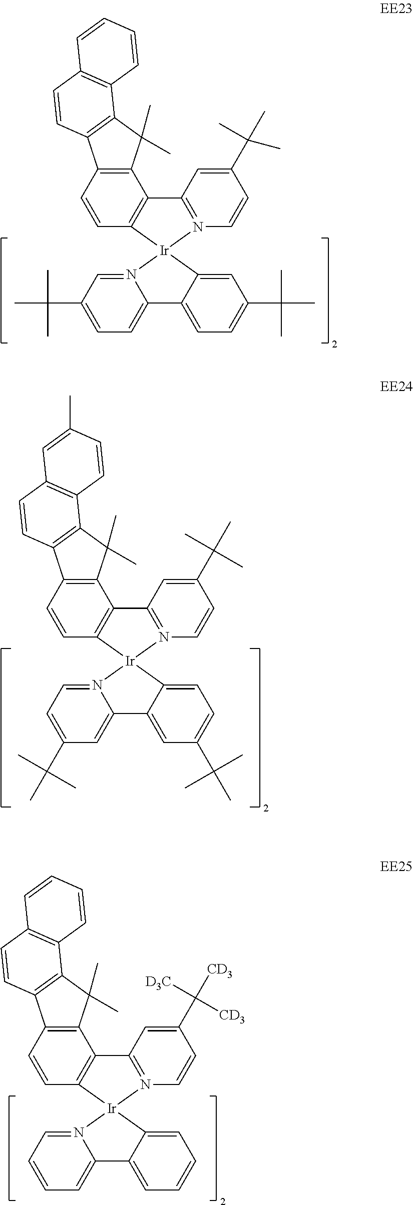

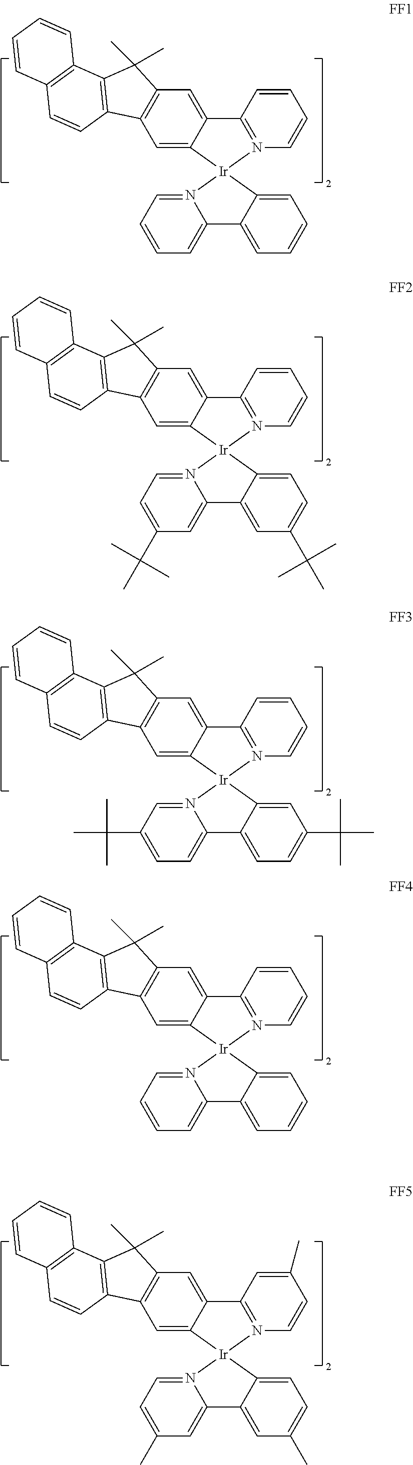

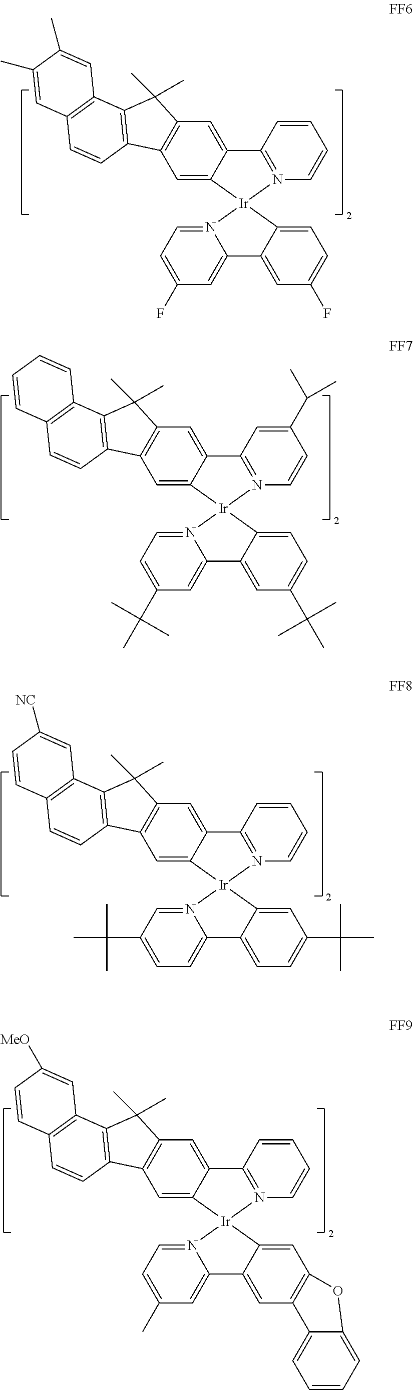

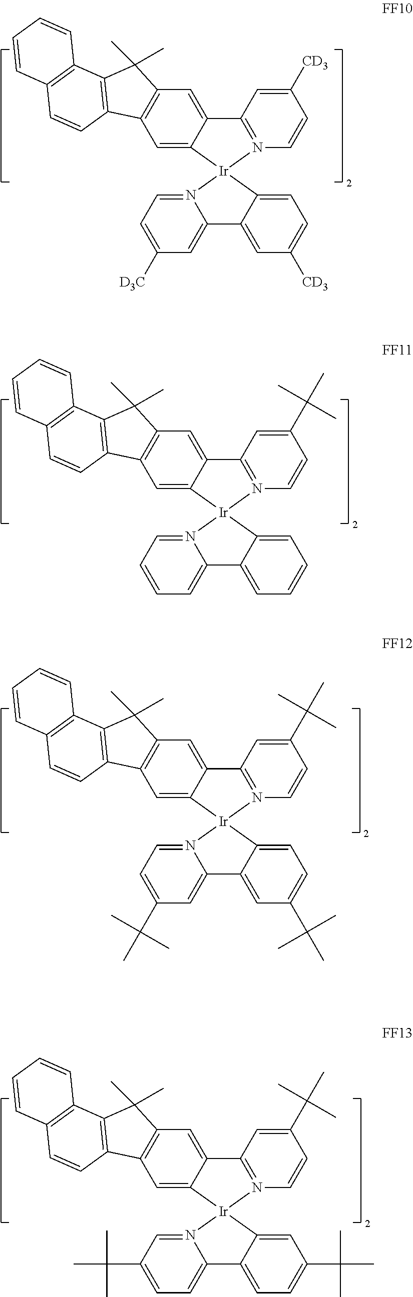

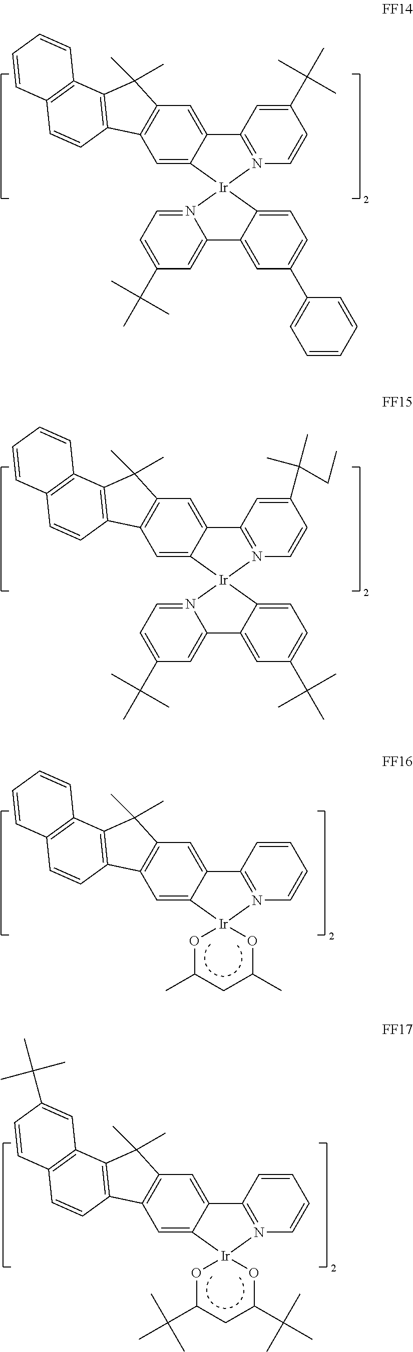

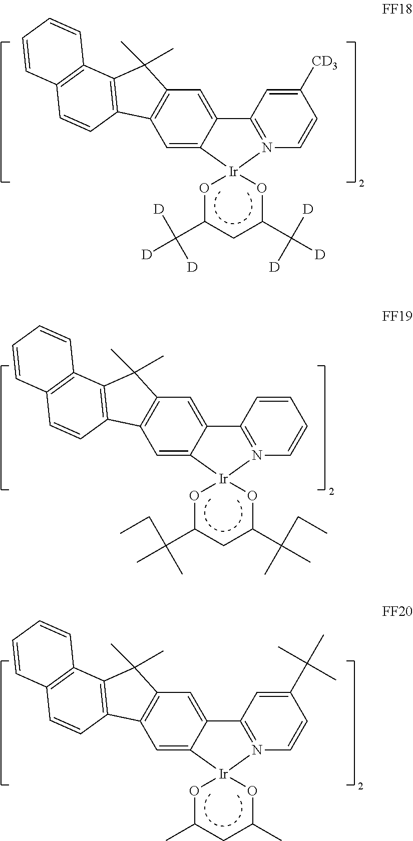

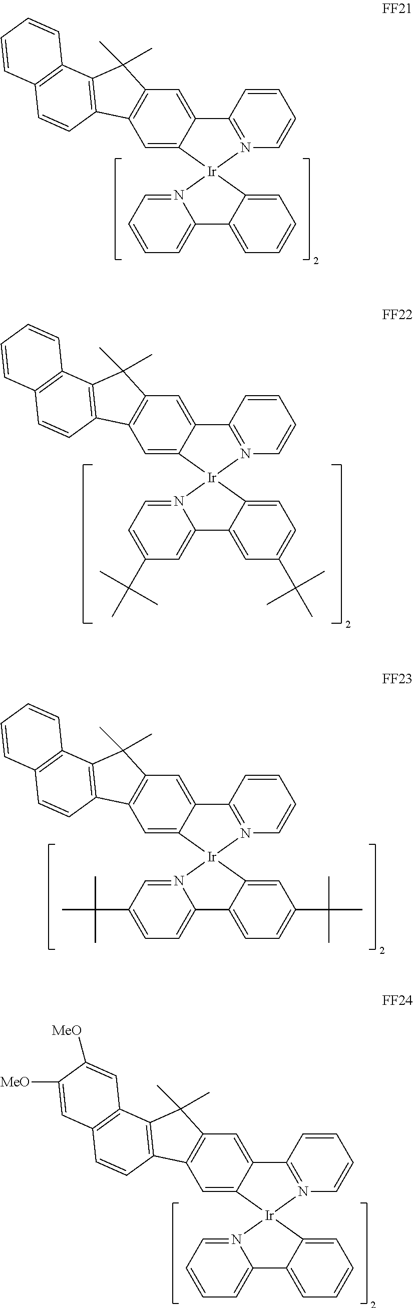

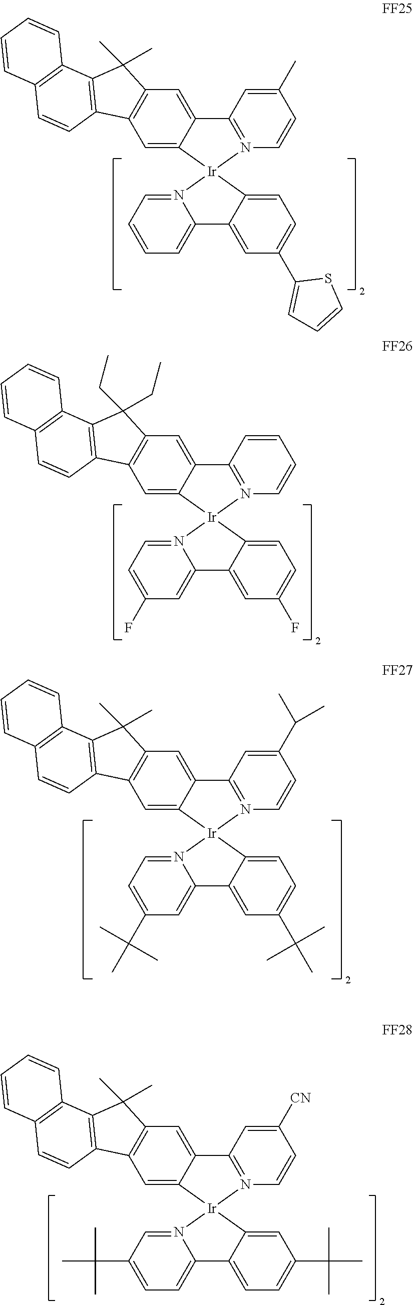

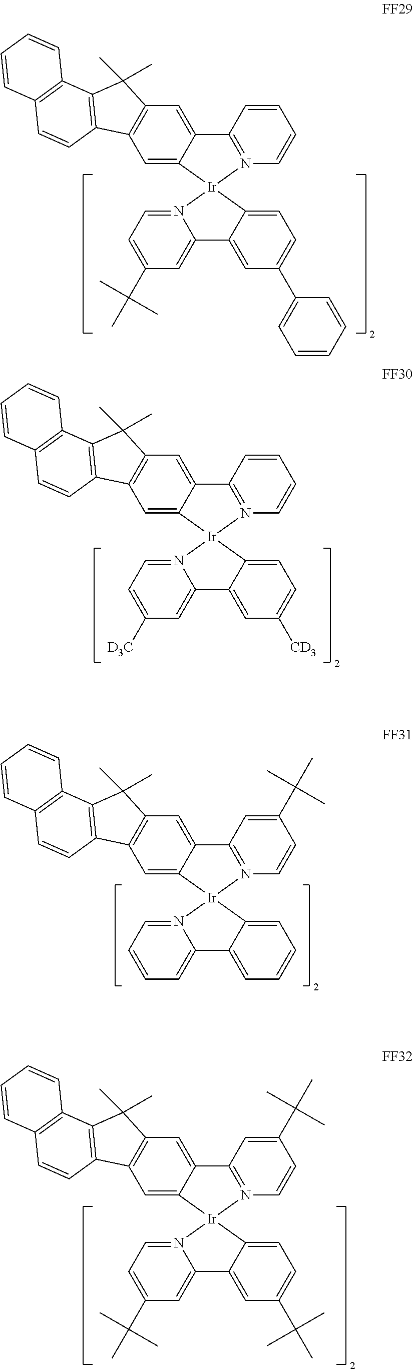

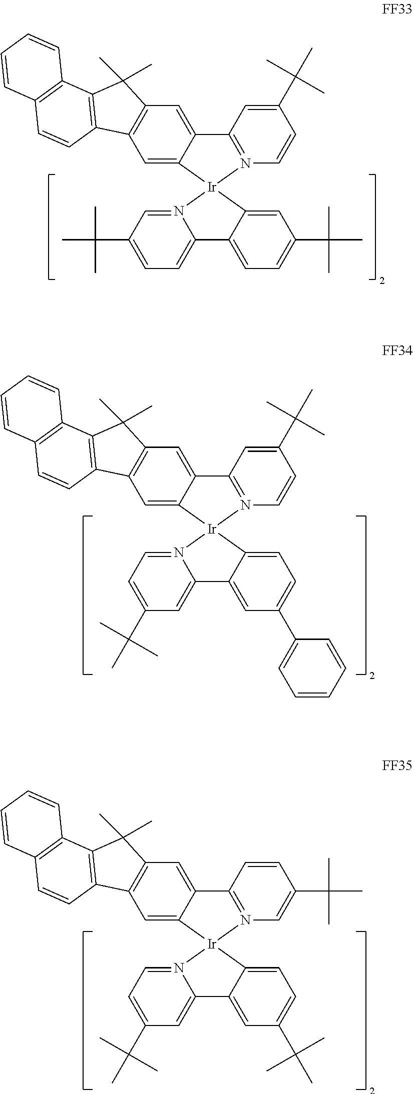

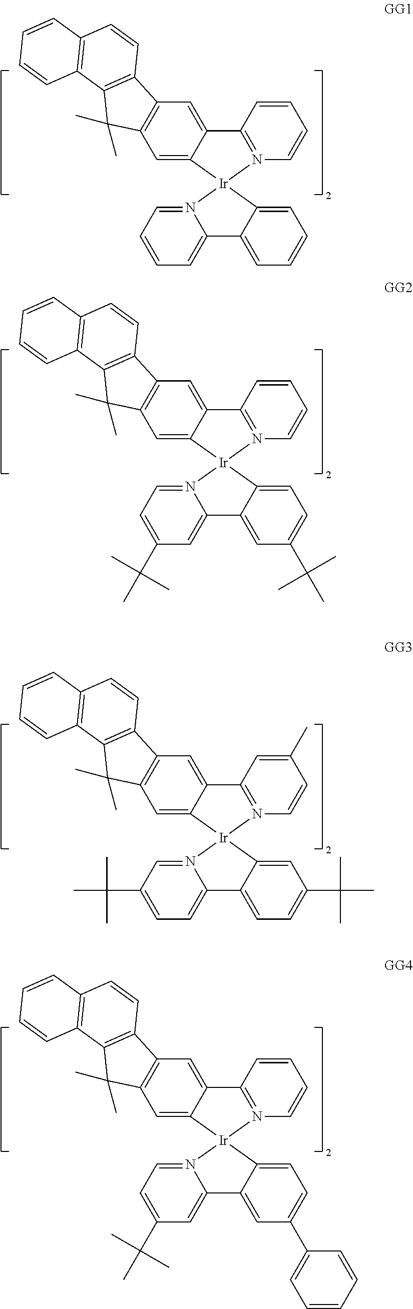

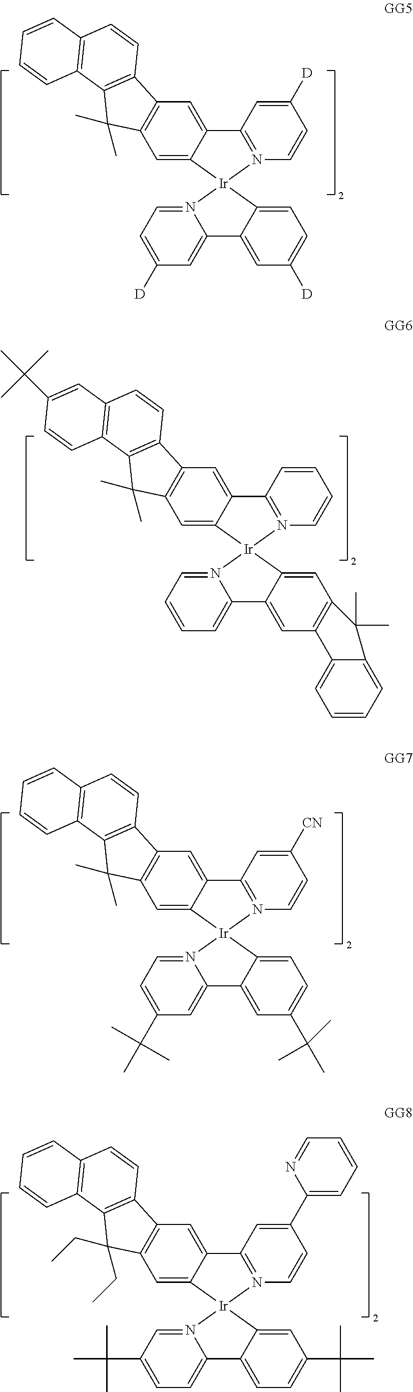

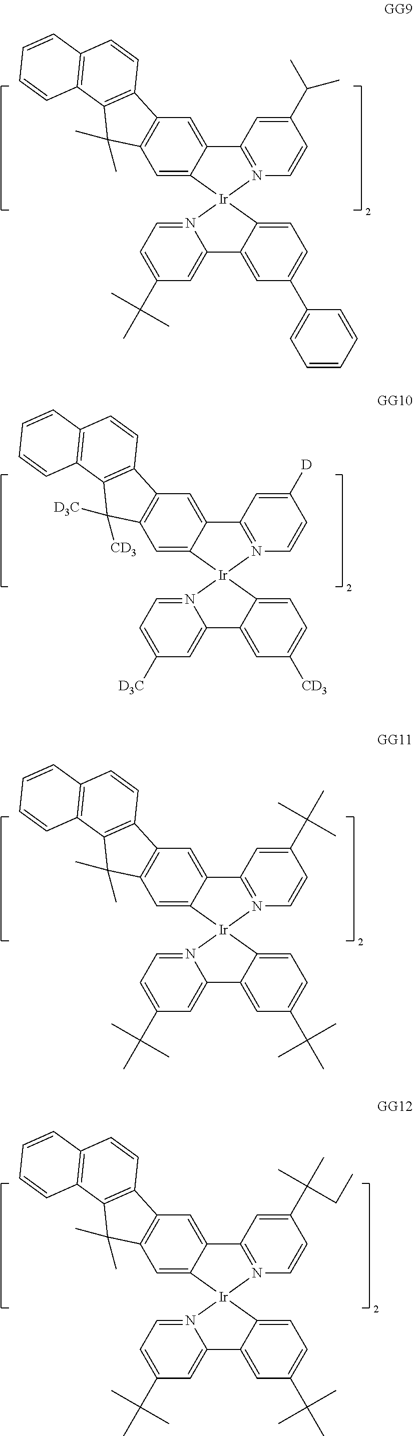

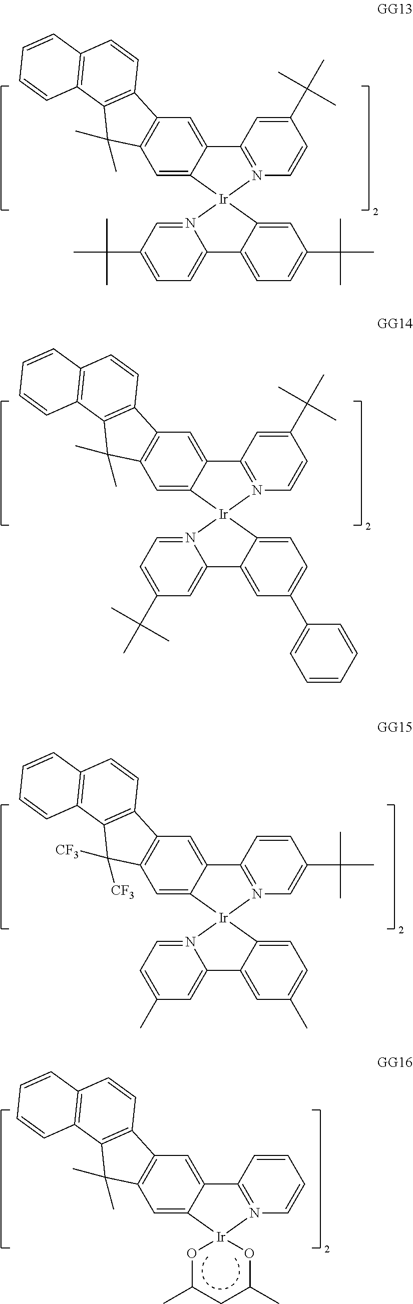

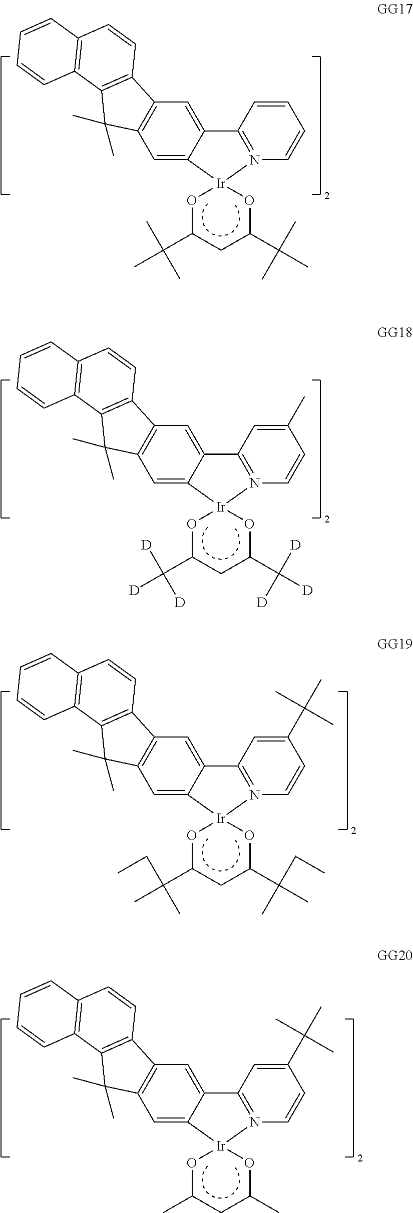

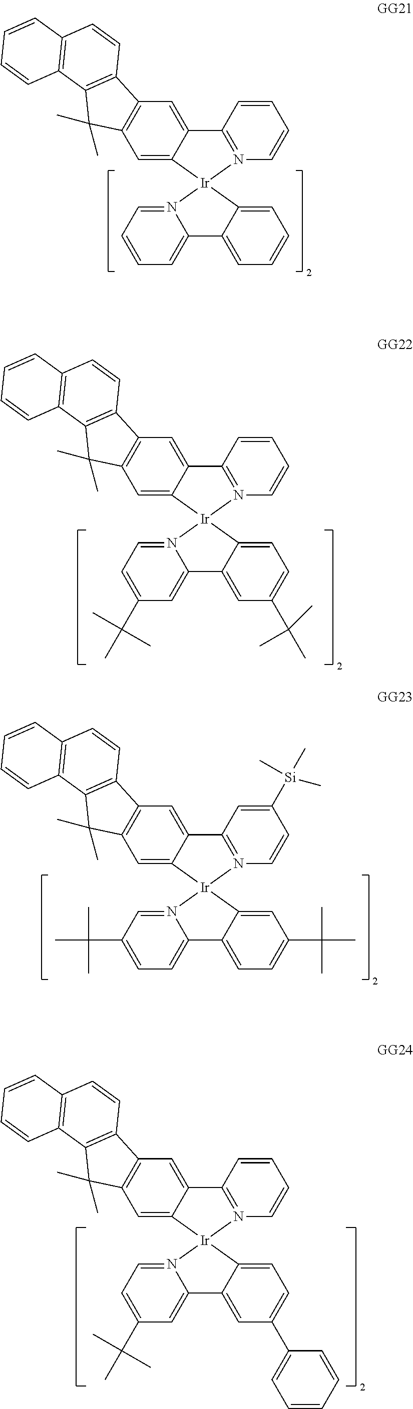

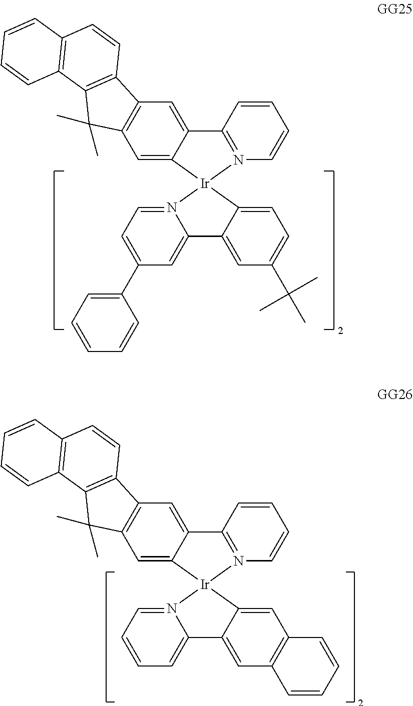

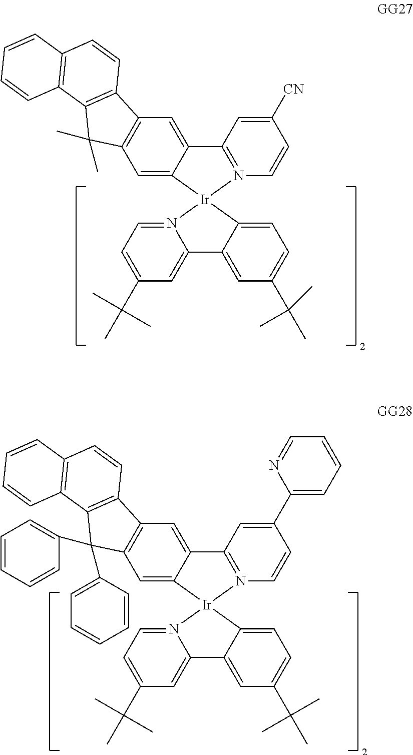

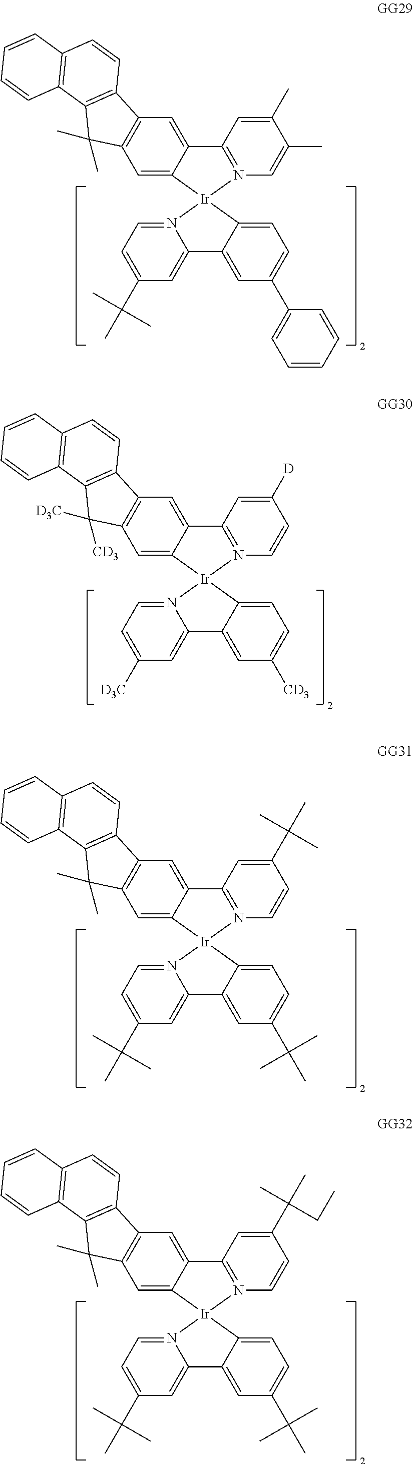

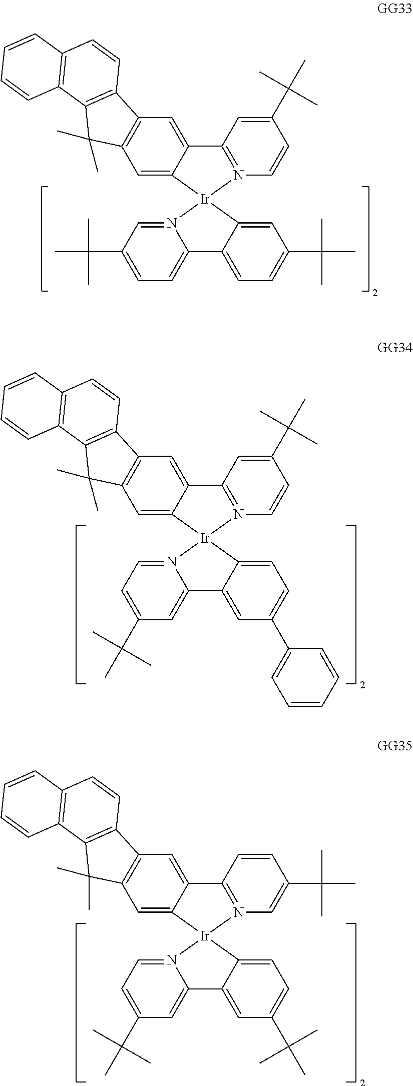

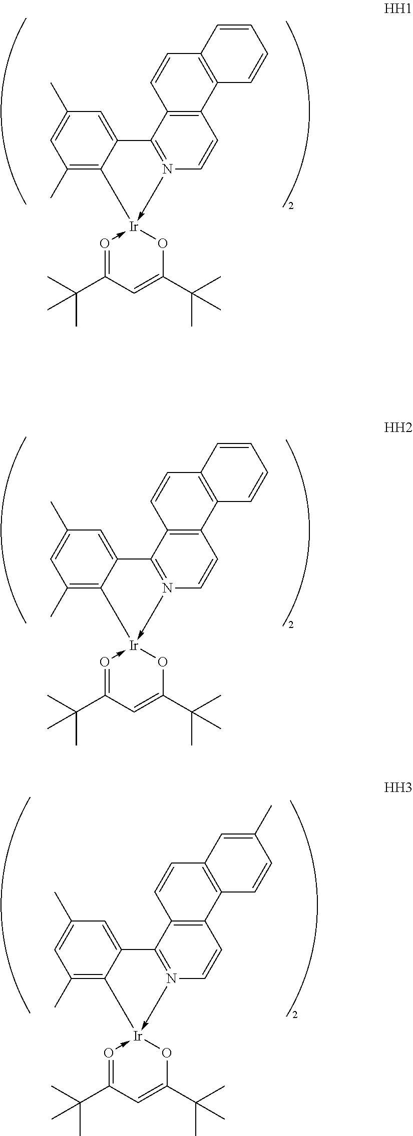

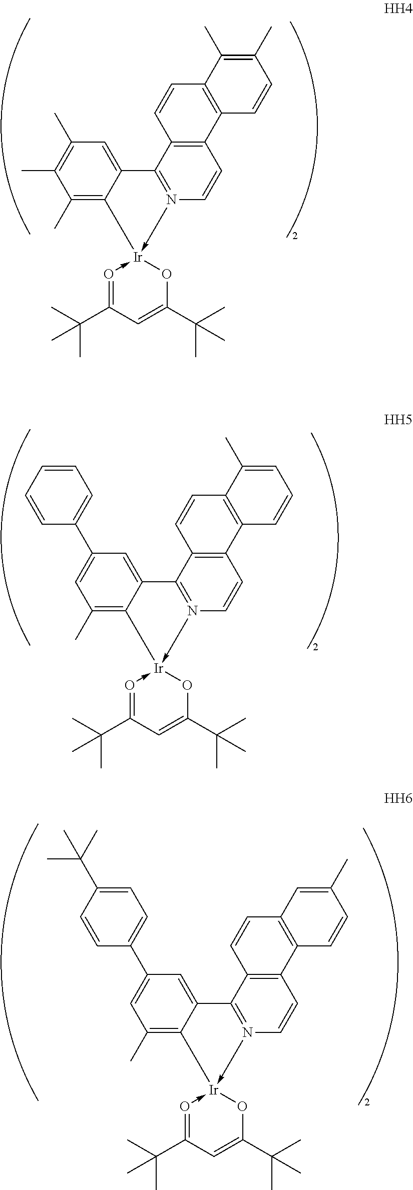

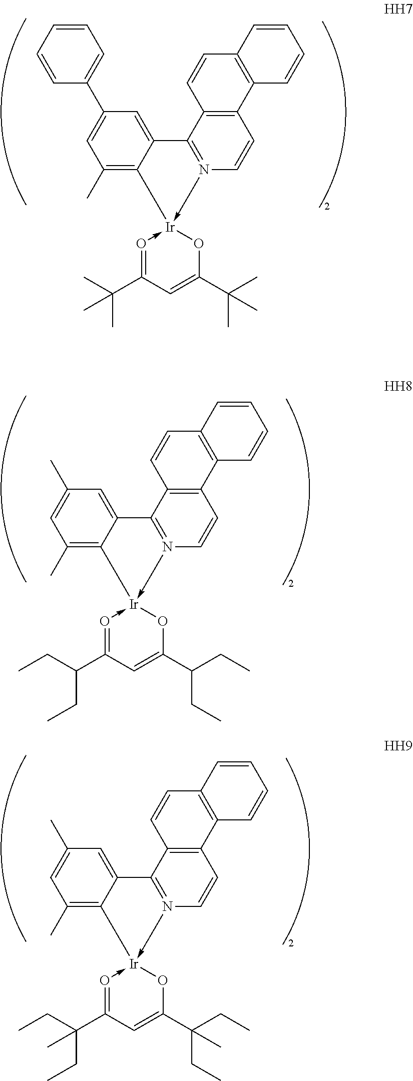

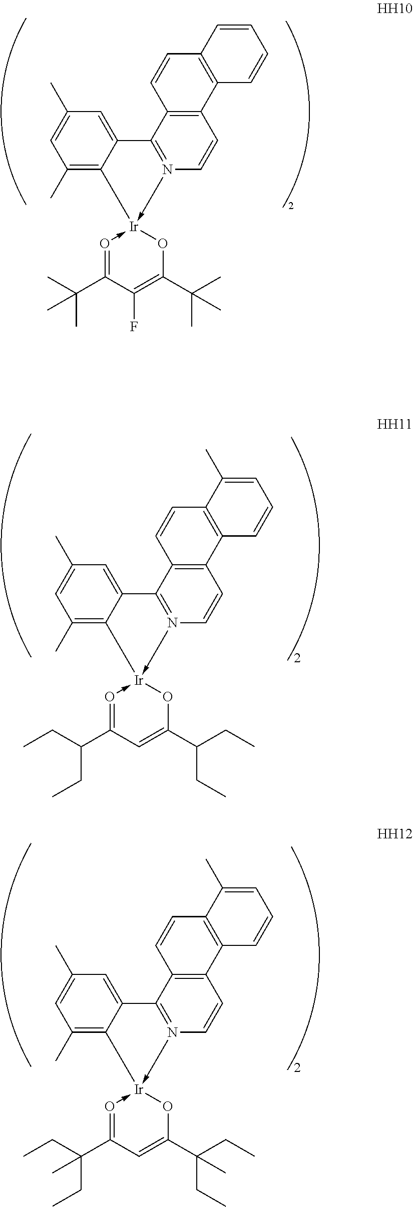

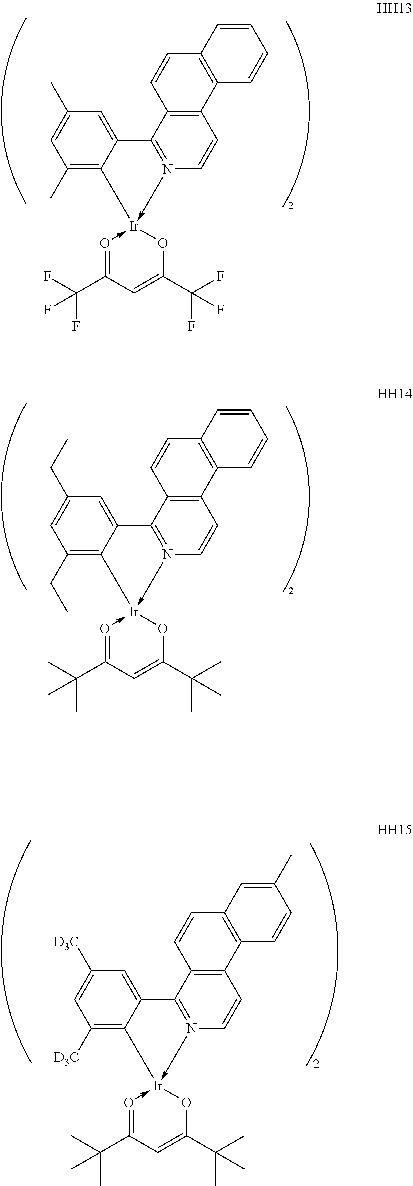

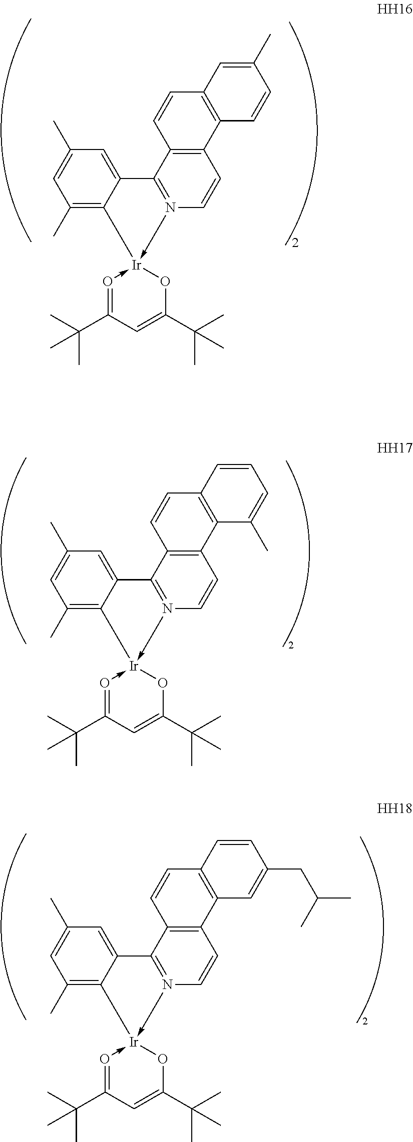

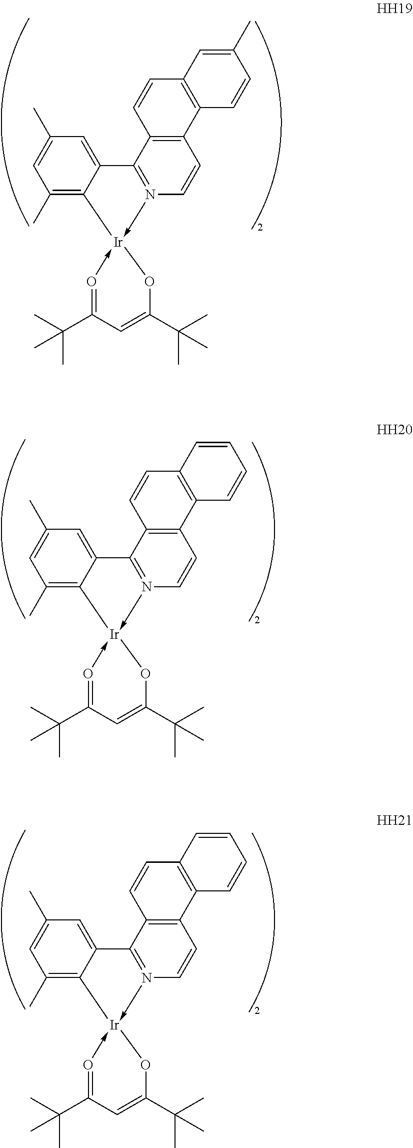

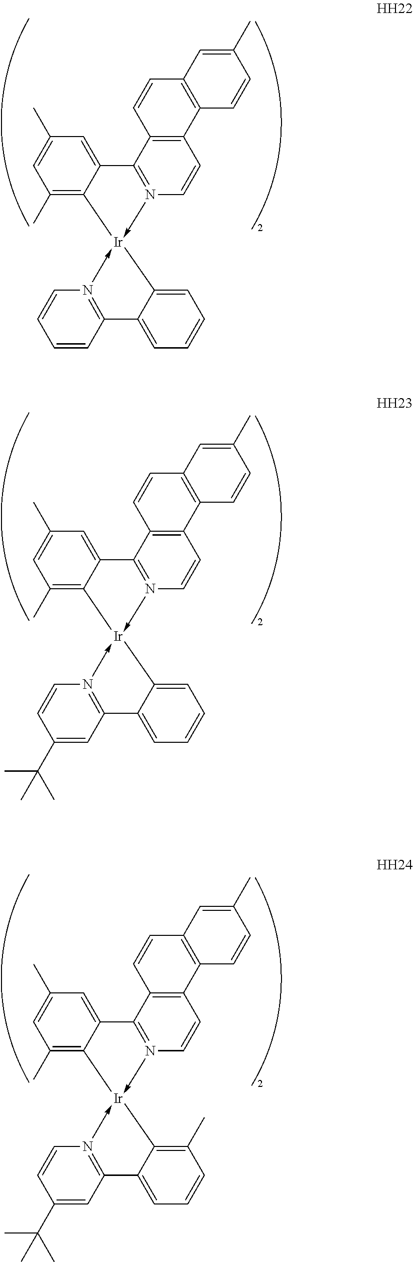

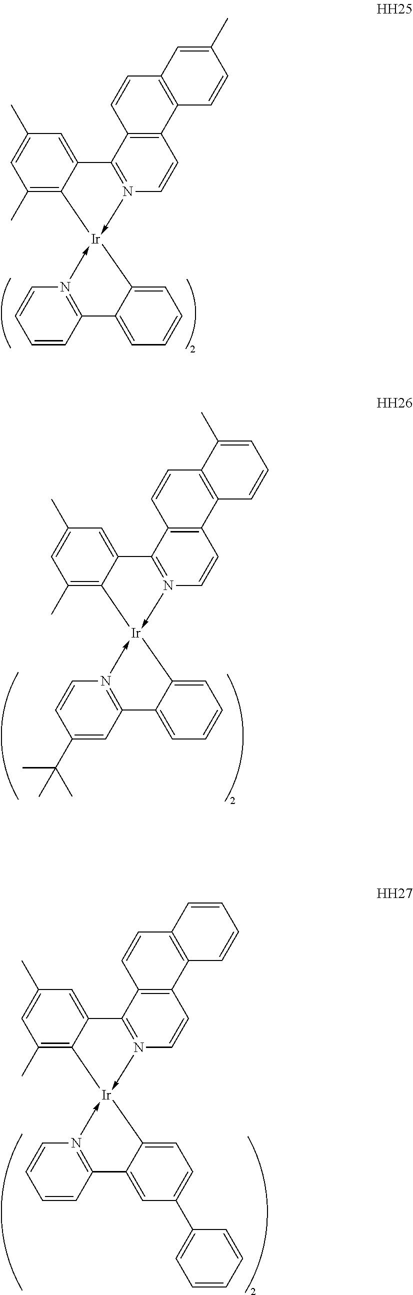

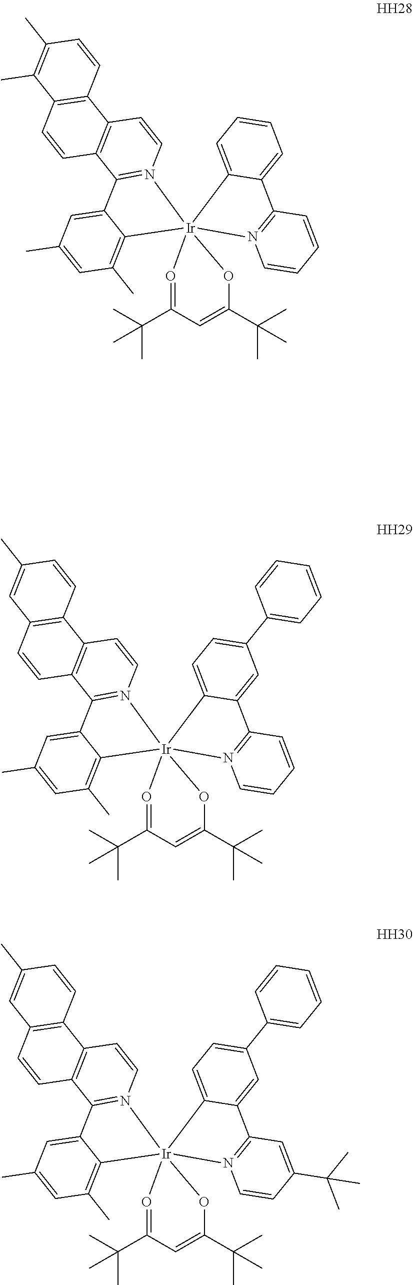

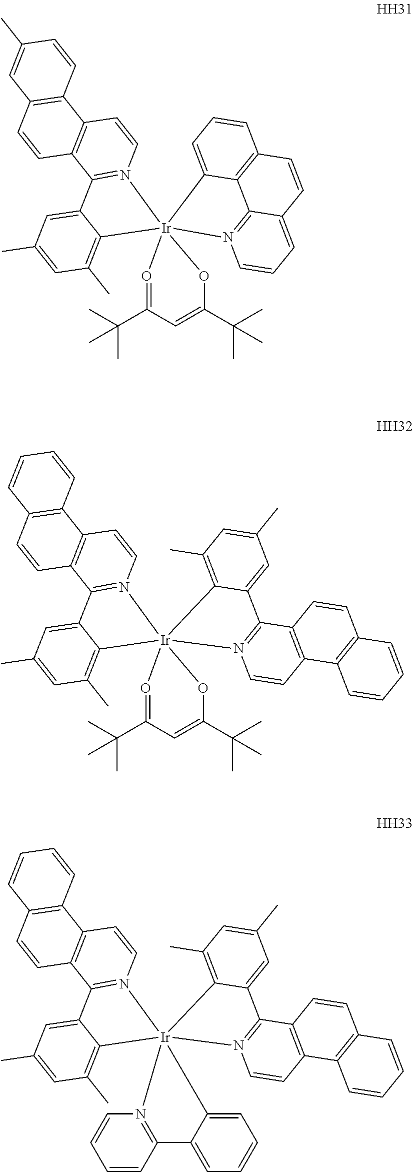

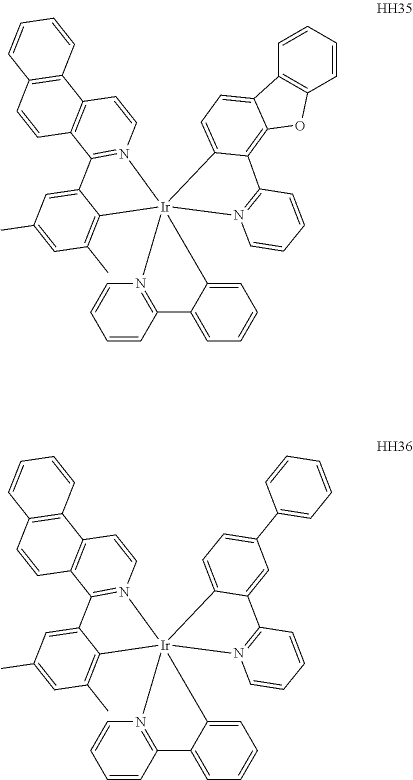

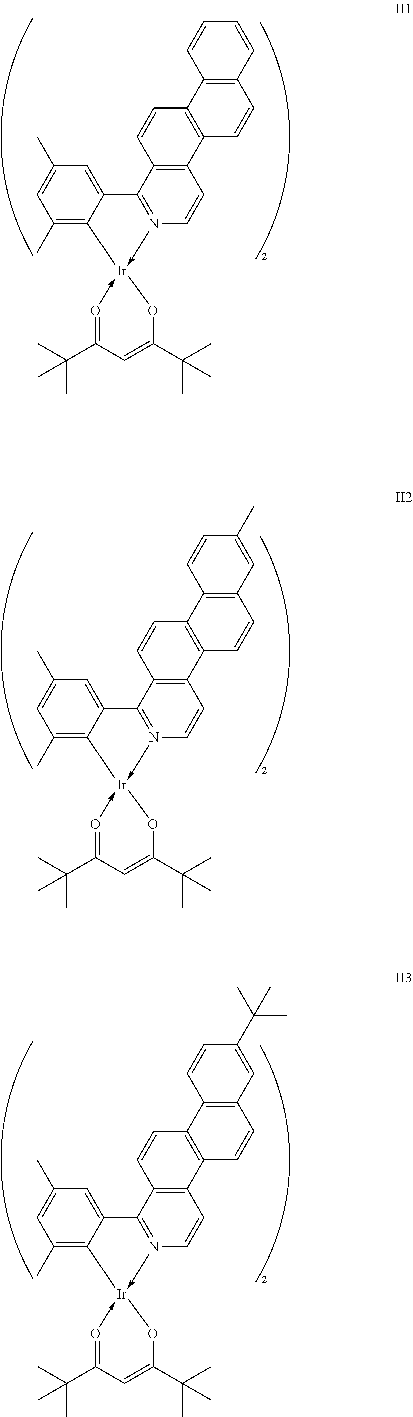

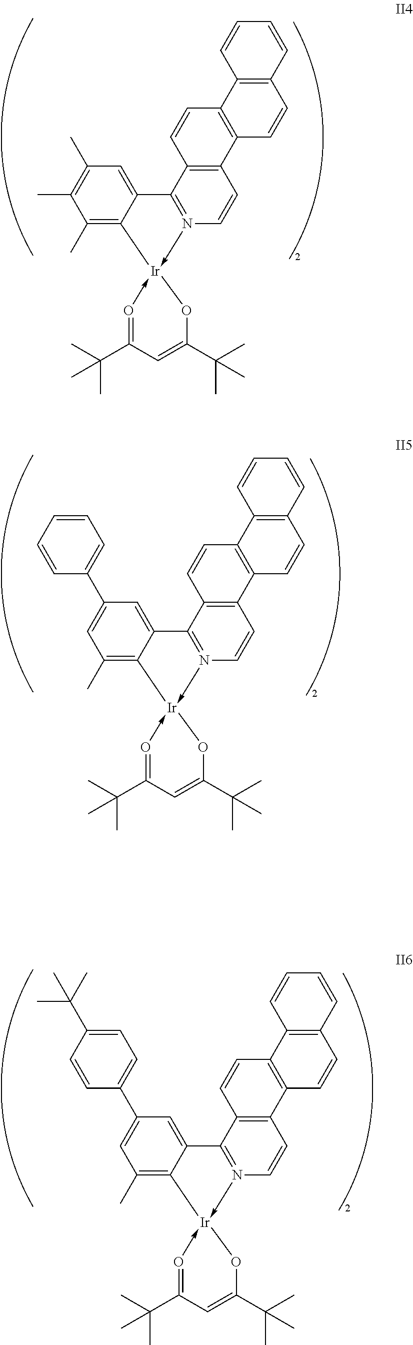

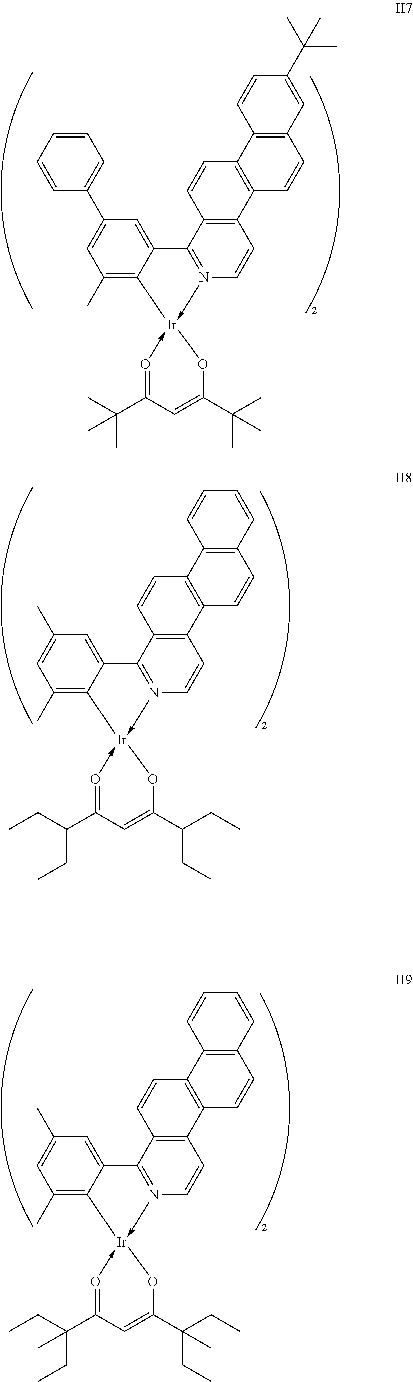

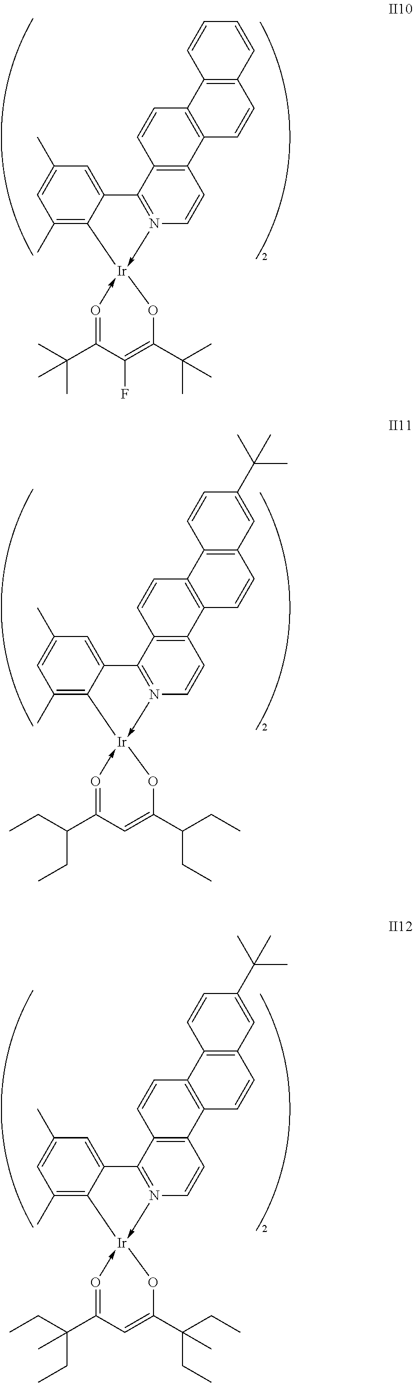

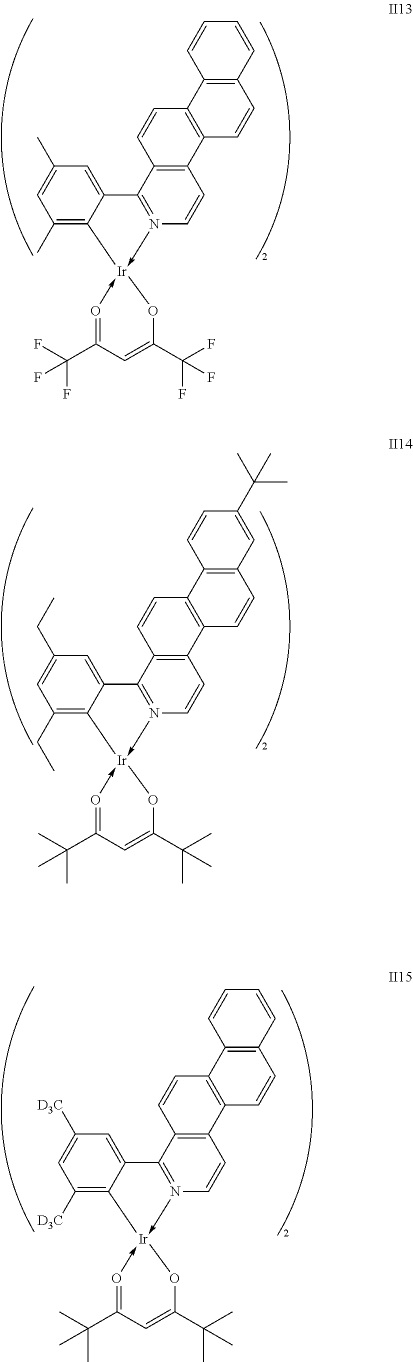

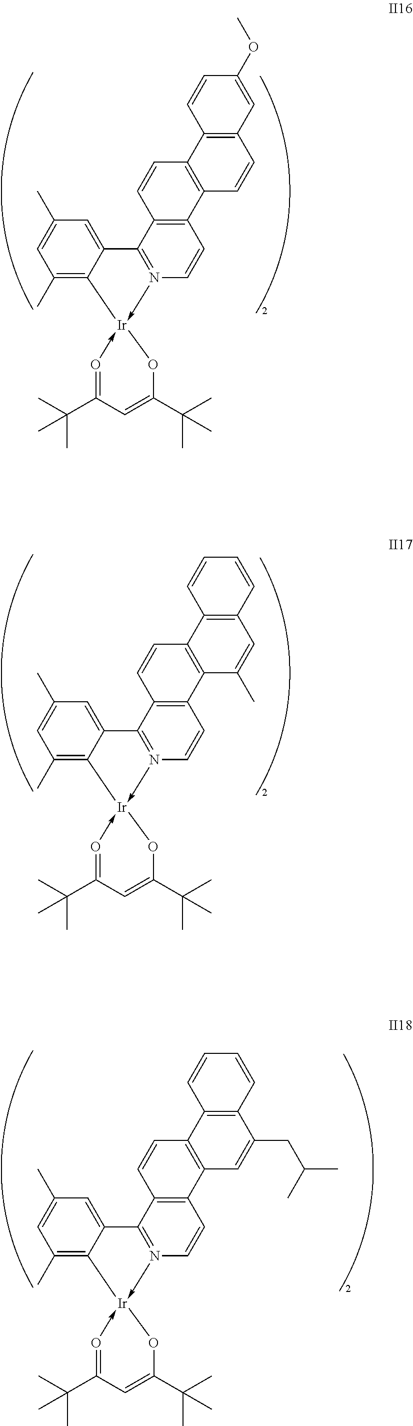

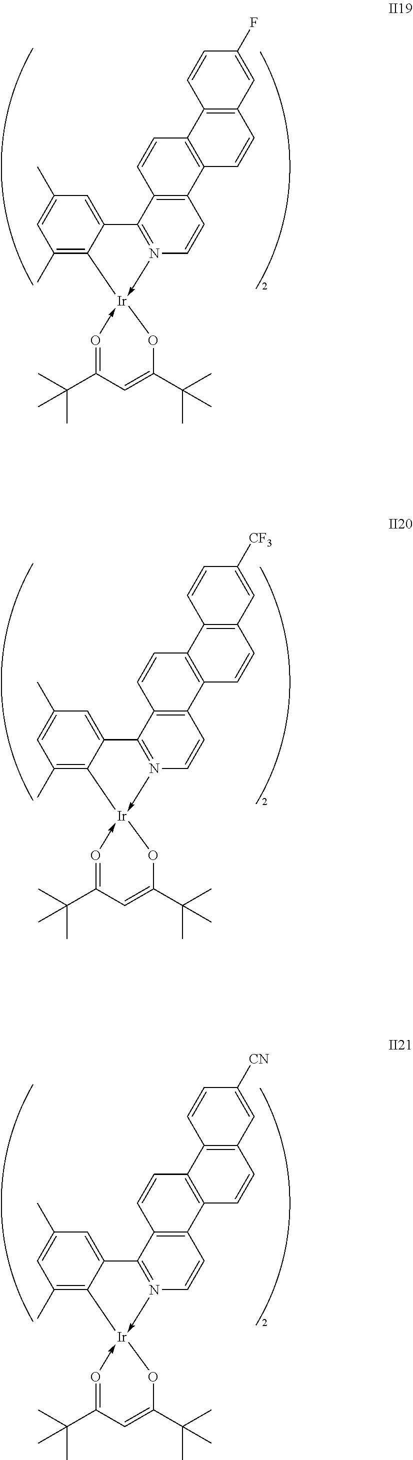

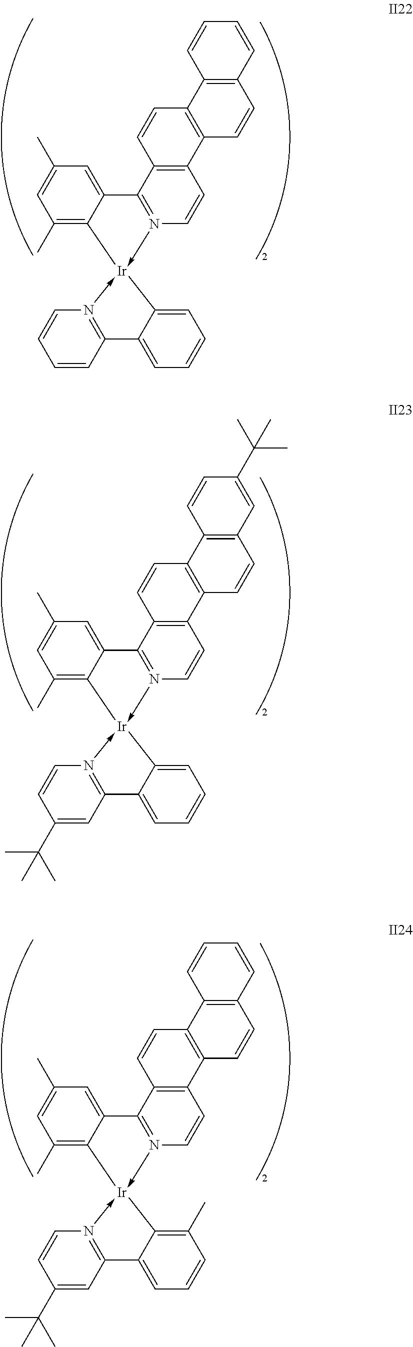

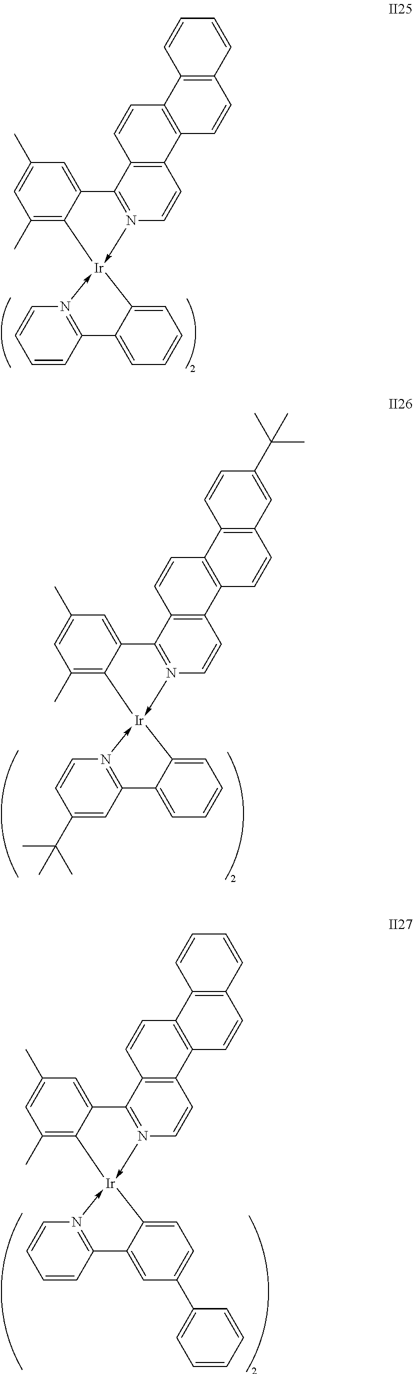

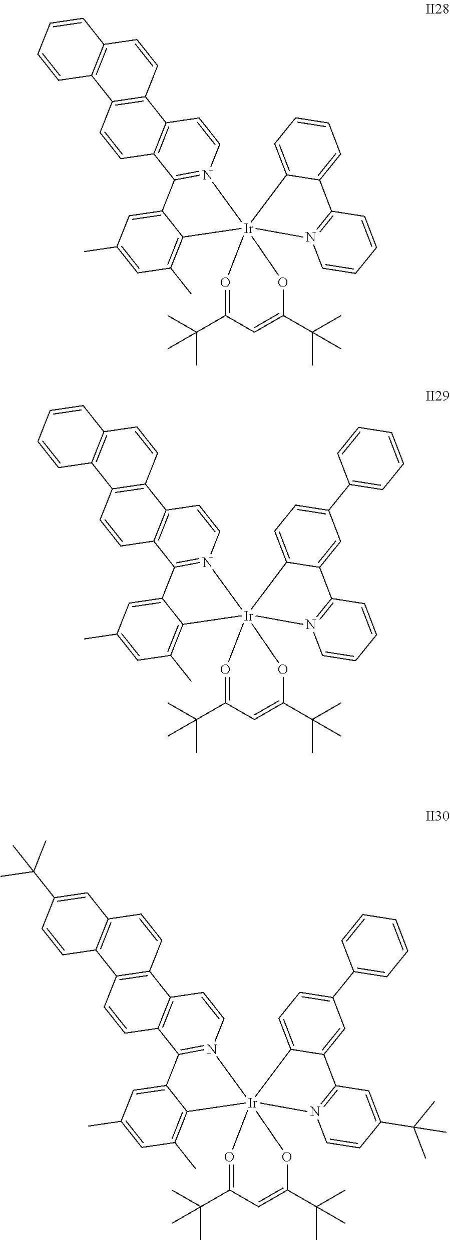

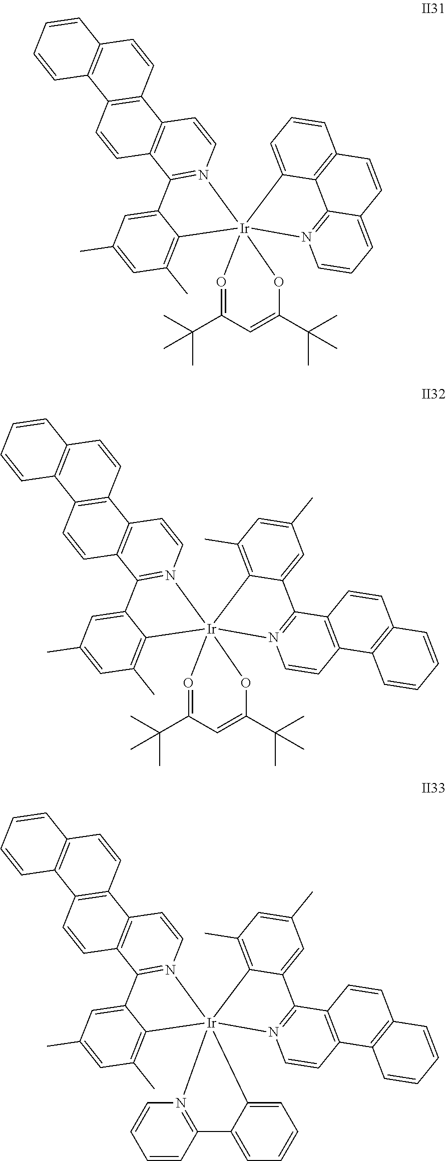

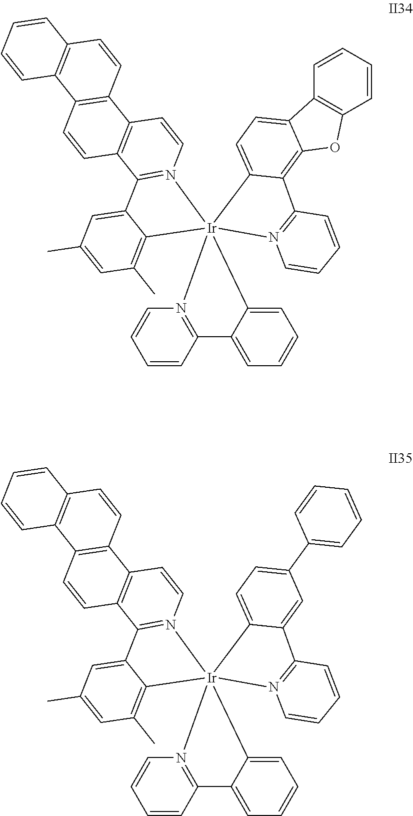

The exemplary compounds belonging to the groups AA and BB are metal complexes in which the partial structure M(L)m is represented by the general formula [Ir-3], and are compounds with at least a phenanthrene ring in the ligand. These compounds particularly have high stability due to their fused rings composed of an SP² hybrid orbital.

The exemplary compounds belonging to the group CC are metal complexes in which the partial structure M(L)m is represented by the general formula [Ir-4], and are compounds with at least a triphenylene ring in the ligand. These compounds particularly have high stability due to their fused rings composed of an SP² hybrid orbital.

The exemplary compounds belonging to the group DD are metal complexes in which the partial structure M(L)m is represented by one of the general formulae [Ir-5] to [Ir-8], and are compounds with at least a dibenzofuran ring, a dibenzothiophene ring, a benzonaphthofuran ring, or a benzonaphthothiophene ring in the ligand. These compounds contain an oxygen atom or a sulfur atom in the fused ring. Abundant lone pairs in these atoms can enhance charge transport ability and make it particularly easy to adjust the carrier balance of the compounds.

The exemplary compounds belonging to the groups EE to GG are metal complexes in which the partial structure M(L)m is represented by one of the general formulae [Ir-6] to [Ir-8], and are compounds with at least a benzofluorene ring in the ligand. These compounds have a substituent at position 9 of the fluorene ring in the direction perpendicular to the in-plane direction of the fluorene ring and can therefore particularly reduce the overlap between fused rings. Thus, the compounds have particularly high sublimability.

The exemplary compounds belonging to the group HH are metal complexes in which the partial structure M(L)m is represented by one of the general formulae [Ir-11] to [Ir-13], and are compounds with at least a benzoisoquinoline ring in the ligand. These compounds contain a N atom in the fused ring, and abundant lone pairs in these atoms and high electronegativity can enhance charge transport ability and make it particularly easy to adjust the carrier balance of the compounds.

The exemplary compounds belonging to the group II are metal complexes in which the partial structure M(L)m is represented by the general formula [Ir-14], and are compounds with at least a naphthoisoquinoline ring in the ligand. These compounds contain a N atom in the fused ring, and abundant lone pairs in these atoms and high electronegativity can enhance charge transport ability and make it particularly easy to adjust the carrier balance of the compounds.

### <Other Compounds>

An organic compound according to the present embodiment can be used as a constituent material of an organic compound layer other than the light-emitting layer constituting the organic light-emitting element according to the present embodiment. More specifically, an organic compound according to the present embodiment may be used as a constituent material of an electron-transport layer, an electron-injection layer, a hole-transport layer, a hole-injection layer, and/or a hole-blocking layer. In such a case, the emission color of the organic light-emitting element is not particularly limited. More specifically, the emission color may be white or a neutral color.

If necessary, an organic light-emitting element according to the present embodiment may also include a known low-molecular-weight or high-molecular-weight hole-injection compound or hole-transport compound, host compound, light-emitting compound, electron-injection compound, or electron-transport compound. Examples of these compounds are described below.

The hole-injection/transport material can be a material with high hole mobility to facilitate the injection of holes from a positive electrode and to transport the injected holes to a light-emitting layer. Furthermore, a material with a high glass transition temperature can be used to reduce degradation of film quality, such as crystallization, in an organic light-emitting element. Examples of the low-molecular-weight or high-molecular-weight material with hole-injection/transport ability include, but are not limited to, triarylamine derivatives, aryl carbazole derivatives, phenylenediamine derivatives, stilbene derivatives, phthalocyanine derivatives, porphyrin derivatives, polyvinylcarbazole, polythiophene, and other electrically conductive polymers. The hole-injection/transport material can also be used for an electron-blocking layer. Specific examples of compounds that can be used as hole-injection/transport materials include, but are not limited to, the following.

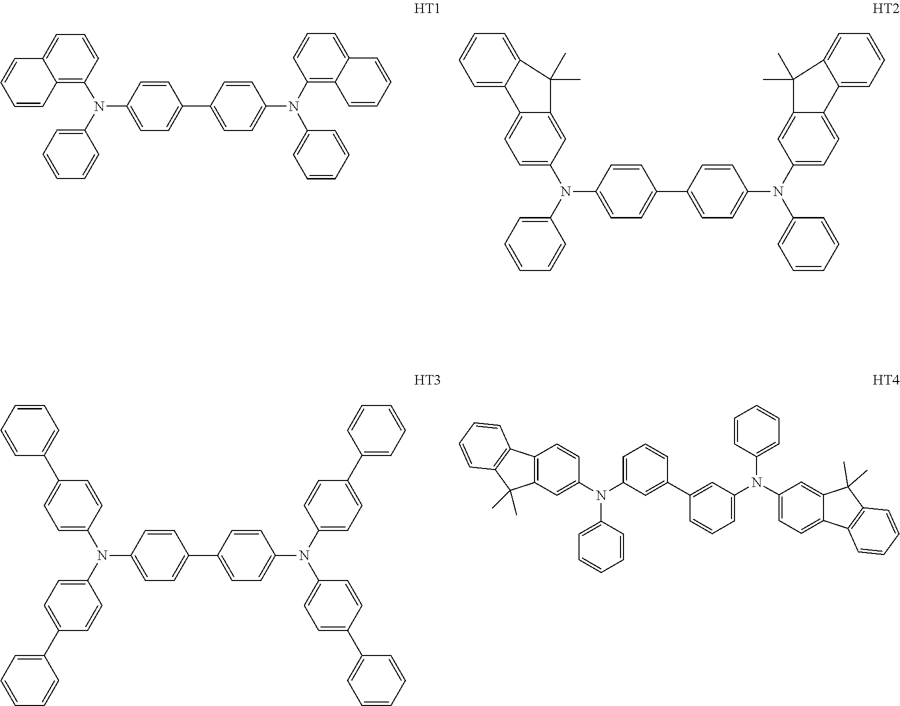 [SMILES: c1ccc(N(c2ccc(-c3ccc(N(c4ccccc4)c4cccc5ccccc45)cc3)cc2)c2cccc3ccccc23)cc1 ; CC1(C)c2ccccc2-c2ccc(N(c3ccccc3)c3ccc(-c4ccc(N(c5ccccc5)c5ccc6c(c5)C(C)(C)c5ccccc5-6)cc4)cc3)cc21 ; c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; CC1(C)c2ccccc2-c2ccc(N(c3ccccc3)c3cccc(-c4cccc(N(c5ccccc5)c5ccc6c(c5)C(C)(C)c5ccccc5-6)c4)c3)cc21]

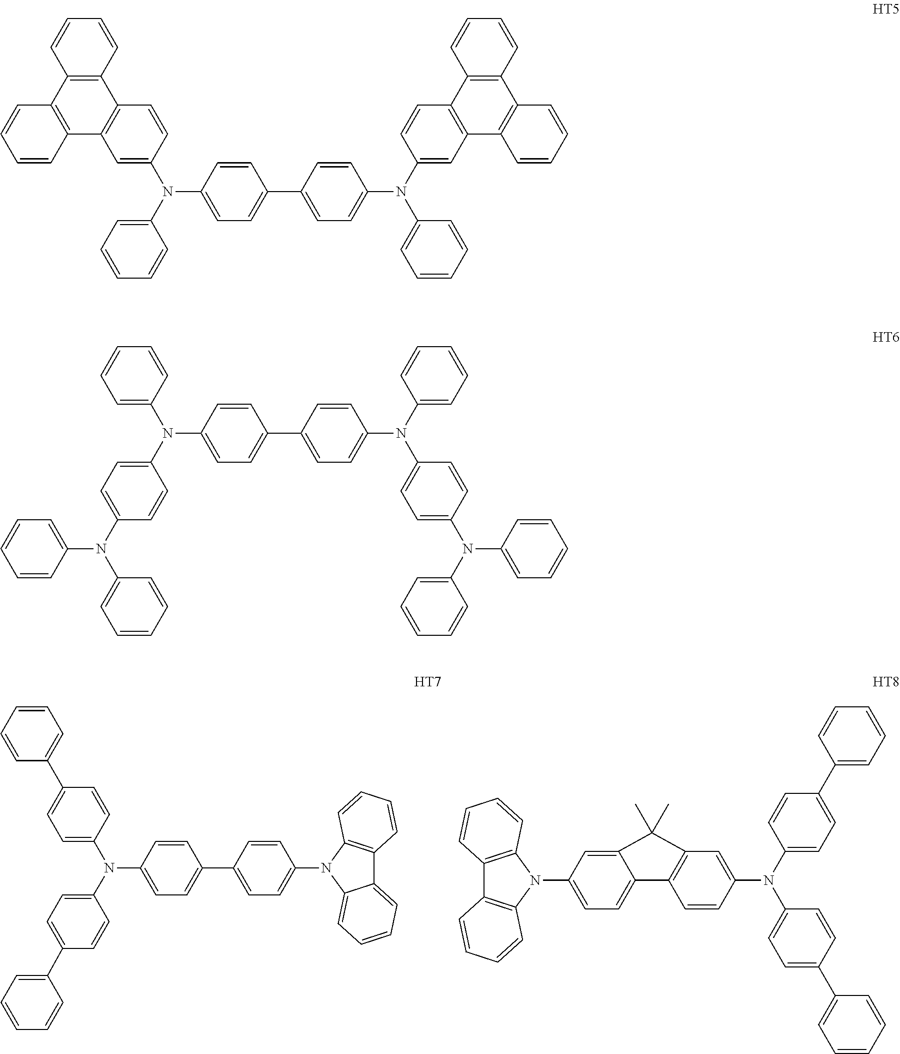 [SMILES: c1ccc(N(c2ccc(-c3ccc(N(c4ccccc4)c4ccc5c6ccccc6c6ccccc6c5c4)cc3)cc2)c2ccc3c4ccccc4c4ccccc4c3c2)cc1 ; c1ccc(N(c2ccccc2)c2ccc(N(c3ccccc3)c3ccc(-c4ccc(N(c5ccccc5)c5ccc(N(c6ccccc6)c6ccccc6)cc5)cc4)cc3)cc2)cc1 ; c1ccc(-c2ccc(N(c3ccc(-c4ccccc4)cc3)c3ccc(-c4ccc(-n5c6ccccc6c6ccccc65)cc4)cc3)cc2)cc1 ; CC1(C)c2cc(N(c3ccc(-c4ccccc4)cc3)c3ccc(-c4ccccc4)cc3)ccc2-c2ccc(-n3c4ccccc4c4ccccc43)cc21]

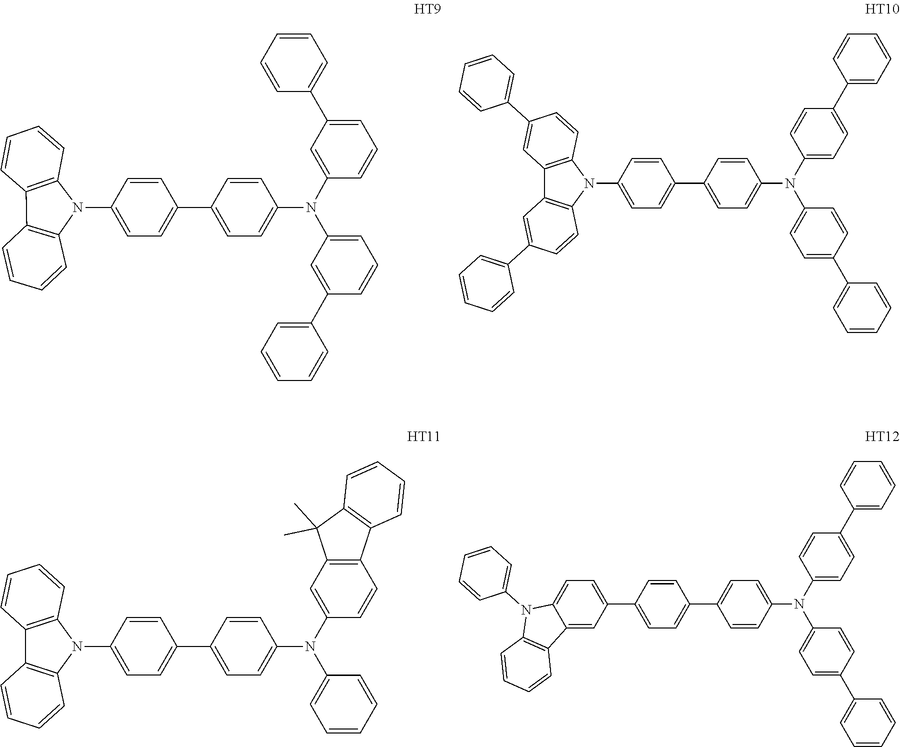 [SMILES: c1ccc(-c2cccc(N(c3ccc(-c4ccc(-n5c6ccccc6c6ccccc65)cc4)cc3)c3cccc(-c4ccccc4)c3)c2)cc1 ; c1ccc(-c2ccc(N(c3ccc(-c4ccccc4)cc3)c3ccc(-c4ccc(-n5c6ccc(-c7ccccc7)cc6c6cc(-c7ccccc7)ccc65)cc4)cc3)cc2)cc1 ; CC1(C)c2ccccc2-c2ccc(N(c3ccccc3)c3ccc(-c4ccc(-n5c6ccccc6c6ccccc65)cc4)cc3)cc21 ; c1ccc(-c2ccc(N(c3ccc(-c4ccccc4)cc3)c3ccc(-c4ccc(-c5ccc6c(c5)c5ccccc5n6-c5ccccc5)cc4)cc3)cc2)cc1]

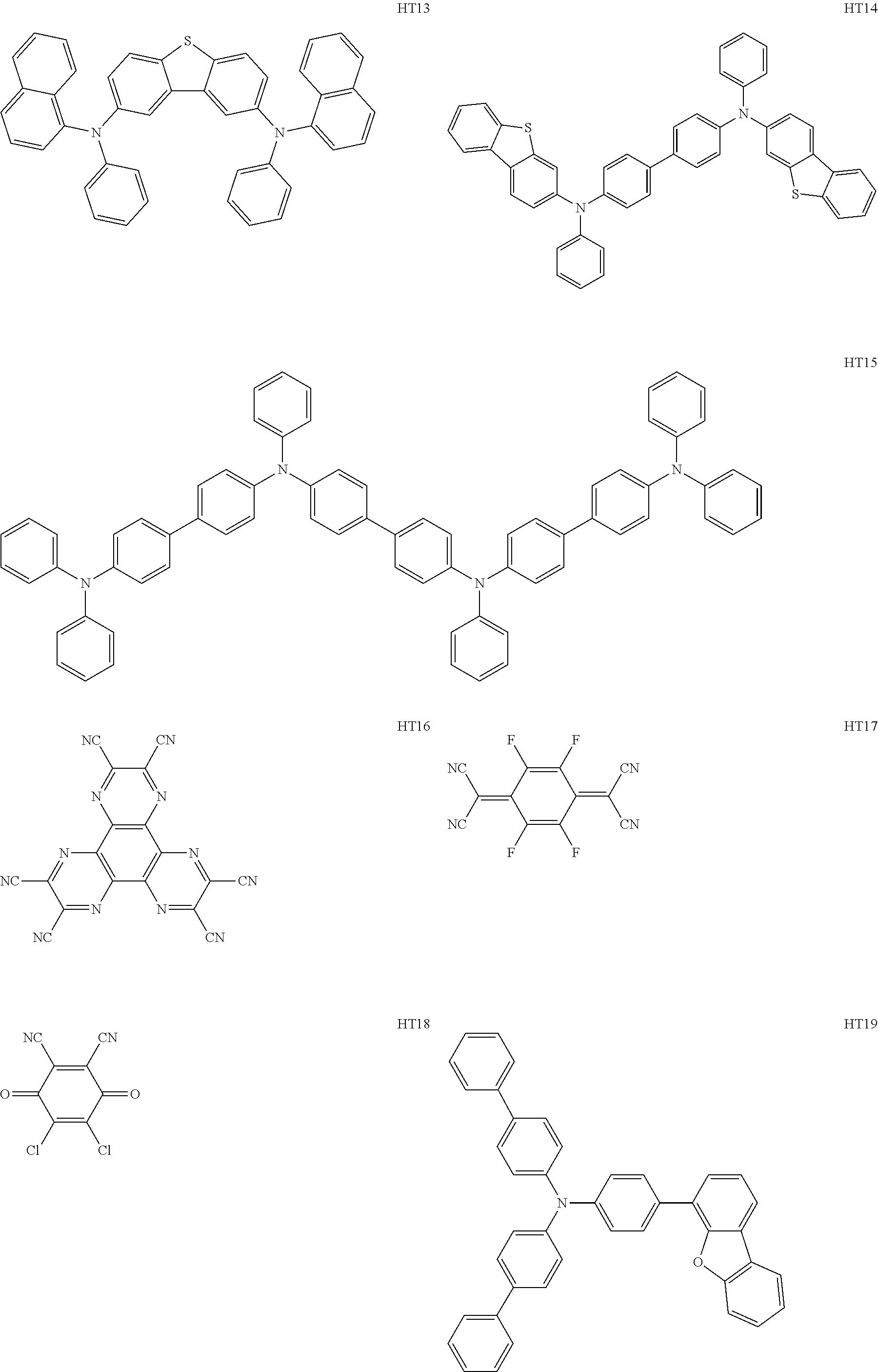

Examples of a light-emitting material mainly related to the light-emitting function include, in addition to the organometallic complexes represented by the general formula [4], fused-ring compounds (for example, fluorene derivatives, naphthalene derivatives, pyrene derivatives, perylene derivatives, tetracene derivatives, anthracene derivatives, rubrene, etc.), quinacridone derivatives, coumarin derivatives, stilbene derivatives, organoaluminum complexes, such as tris(8-quinolinolato)aluminum, iridium complexes, platinum complexes, rhenium complexes, copper complexes, europium complexes, ruthenium complexes, and polymer derivatives, such as poly(phenylene vinylene) derivatives, polyfluorene derivatives, and polyphenylene derivatives. Specific examples of compounds that can be used as light-emitting materials include, but are not limited to, the following.

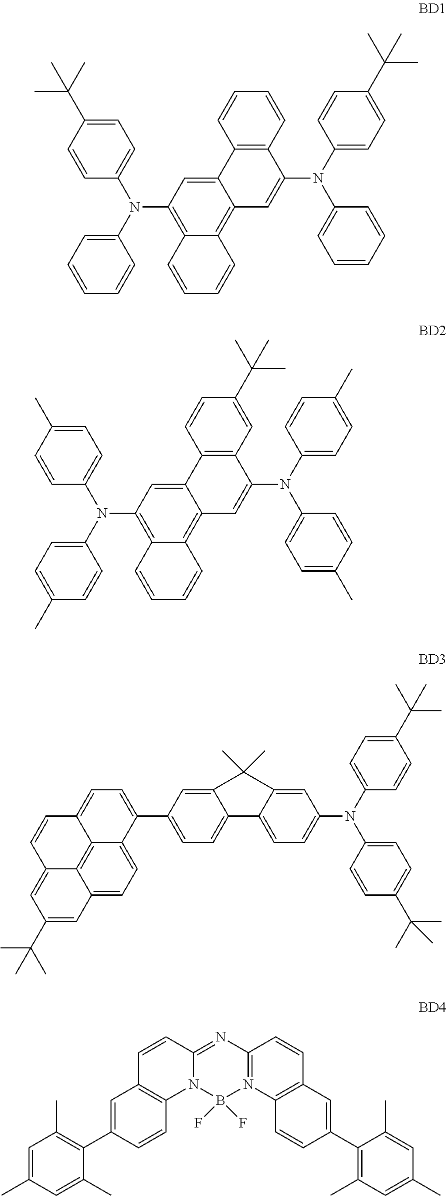

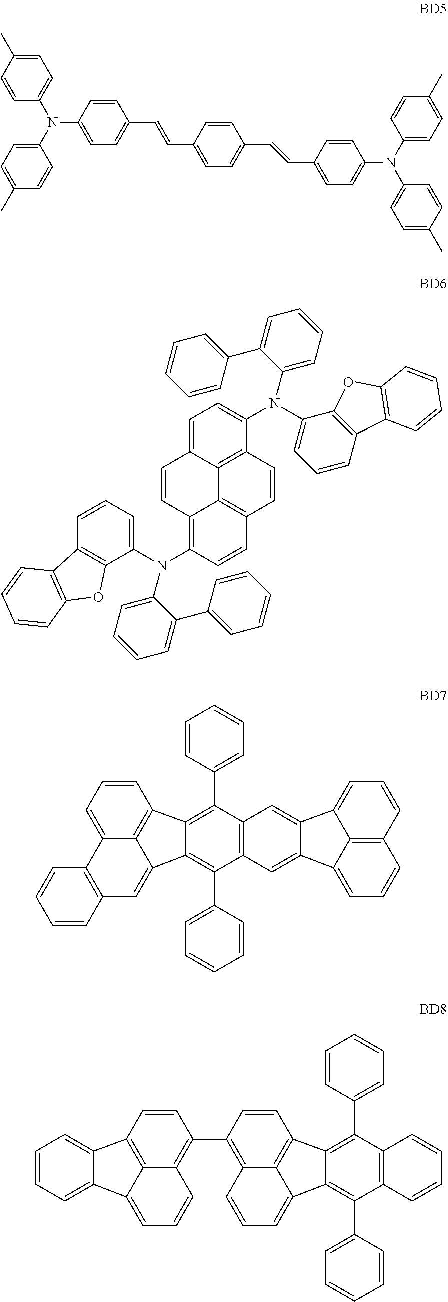

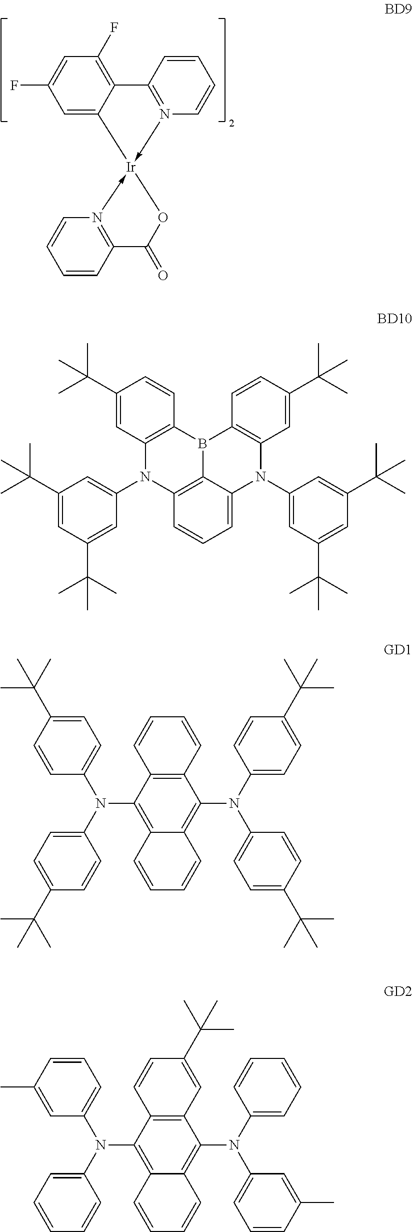

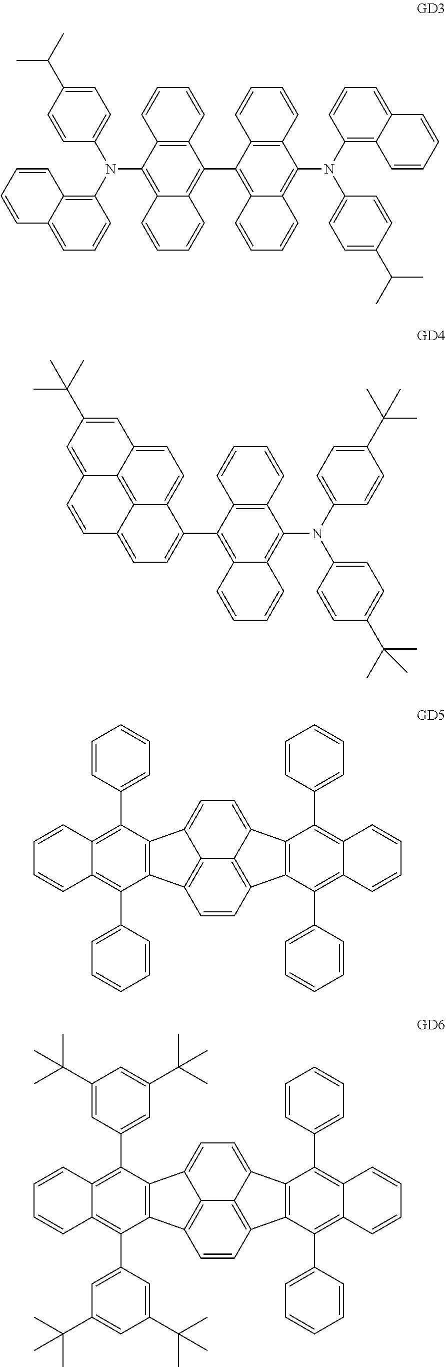

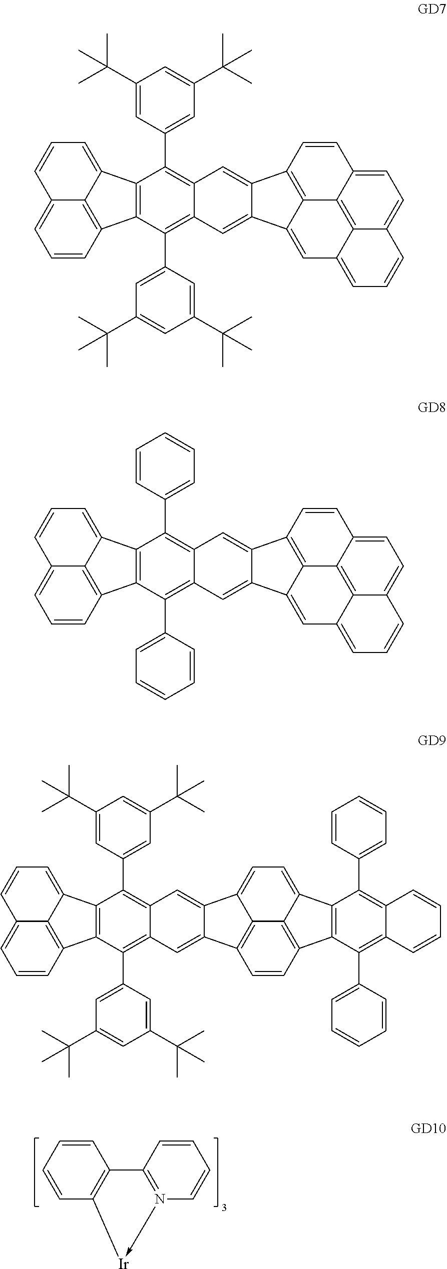

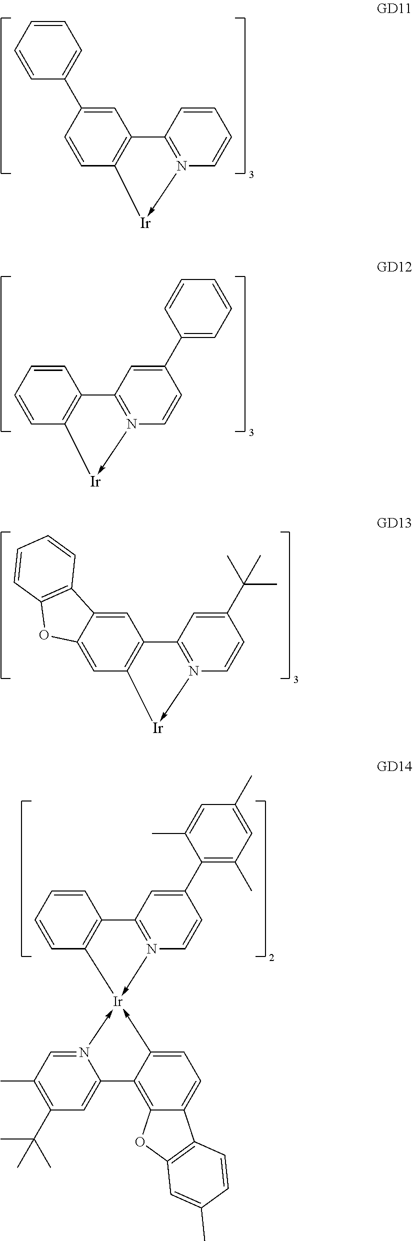

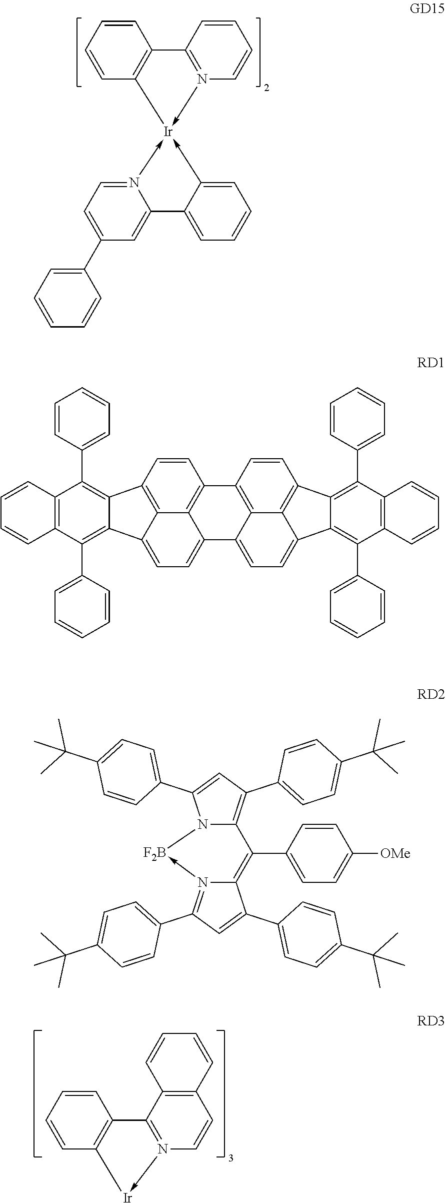

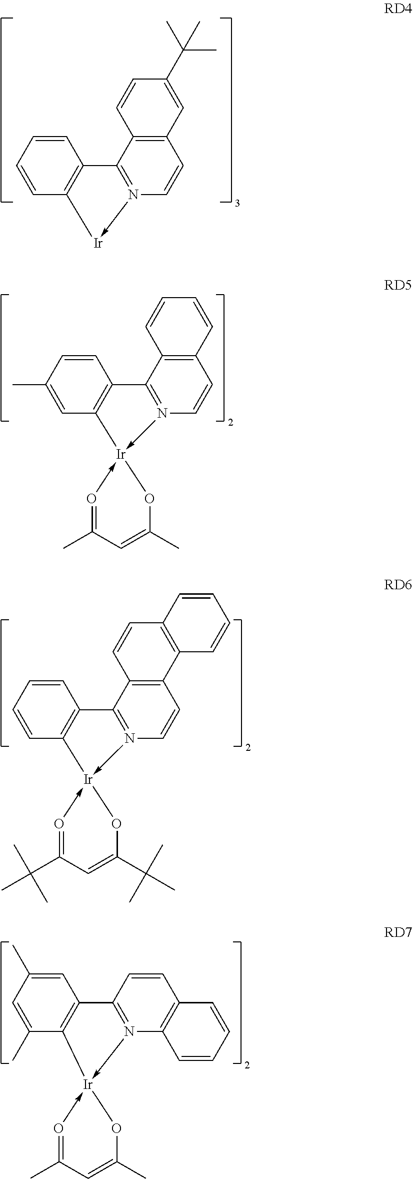

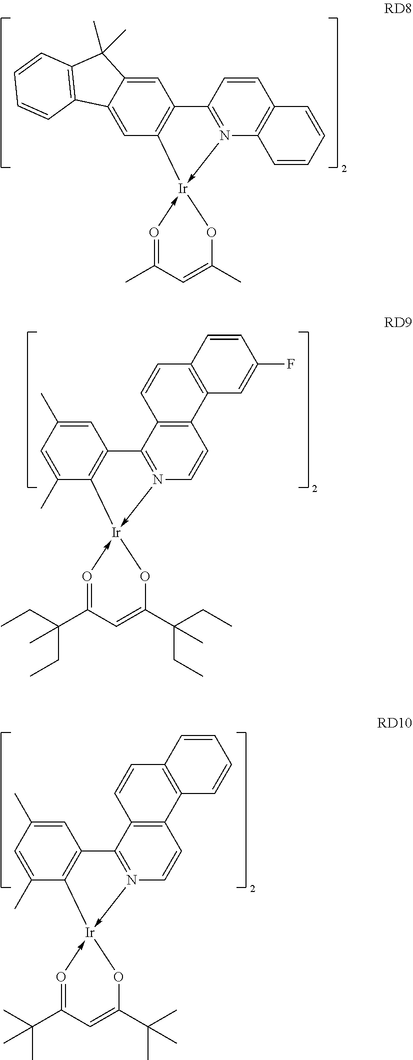

A compound other than the organic compound according to the present embodiment may be contained as a third component as a light-emitting layer host or a light-emitting assist material contained in a light-emitting layer. Examples of the third component include aromatic hydrocarbon compounds and derivatives thereof, carbazole derivatives, azine derivatives, xanthone derivatives, dibenzofuran derivatives, dibenzothiophene derivatives, organoaluminum complexes, such as tris(8-quinolinolato) aluminum, and organoberyllium complexes.

In particular, the assist material can be a material with a carbazole skeleton, a material with an azine ring, such as a diazine ring or a triazine ring, in the skeleton, or a material with xanthone in the skeleton. This is because these materials have high electron-donating ability and electron-withdrawing ability, and the HOMO and LUMO levels can be easily adjusted. An organic compound according to the present embodiment has a structure with a triphenylene skeleton and a nitrogen-containing chalcogen heterocyclic skeleton and therefore has a wide band gap to some extent. In particular, a material with a skeleton that can adjust the HOMO or LUMO level can serve as an assist material. Such an assist material in combination with an organic compound according to the present embodiment can achieve a good carrier balance.

Specific examples of a compound that can be used as a light-emitting layer host or a light-emitting assist material in a light-emitting layer include, but are not limited to, the following. Among the following specific examples, materials with a carbazole skeleton that can serve as assist materials are EM32 to EM38. Materials with an azine ring in the skeleton that can serve as assist materials are EM35 to EM40. Materials with xanthone in the skeleton that can serve as assist materials are EM28 and EM30.

When the host material is a hydrocarbon compound, a compound according to the present embodiment can easily trap an electron or hole and is effective in improving efficiency. The hydrocarbon compound is a compound composed of only carbon and hydrogen and includes EM1 to EM12 and EM16 to EM27 among the following exemplary compounds.

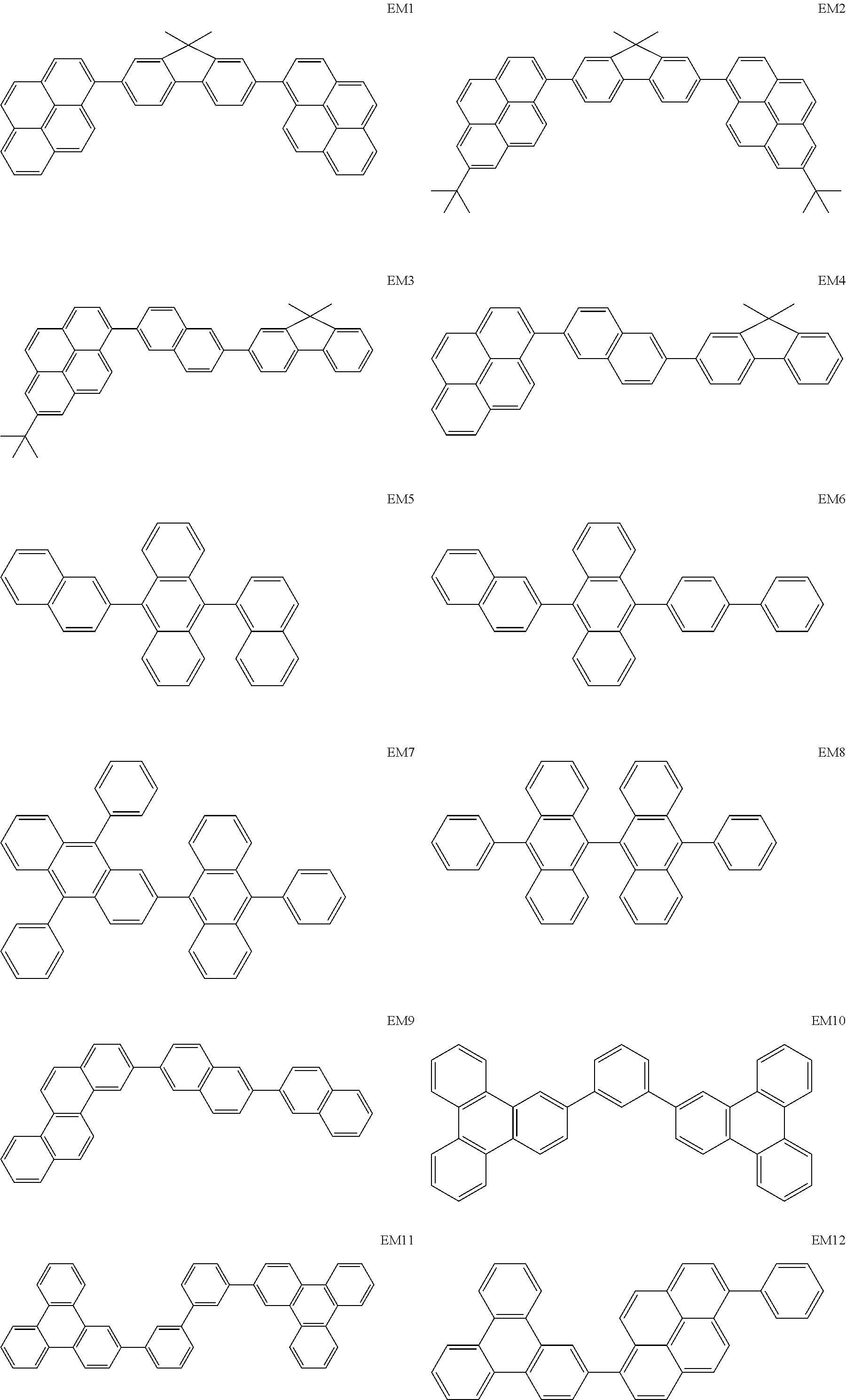

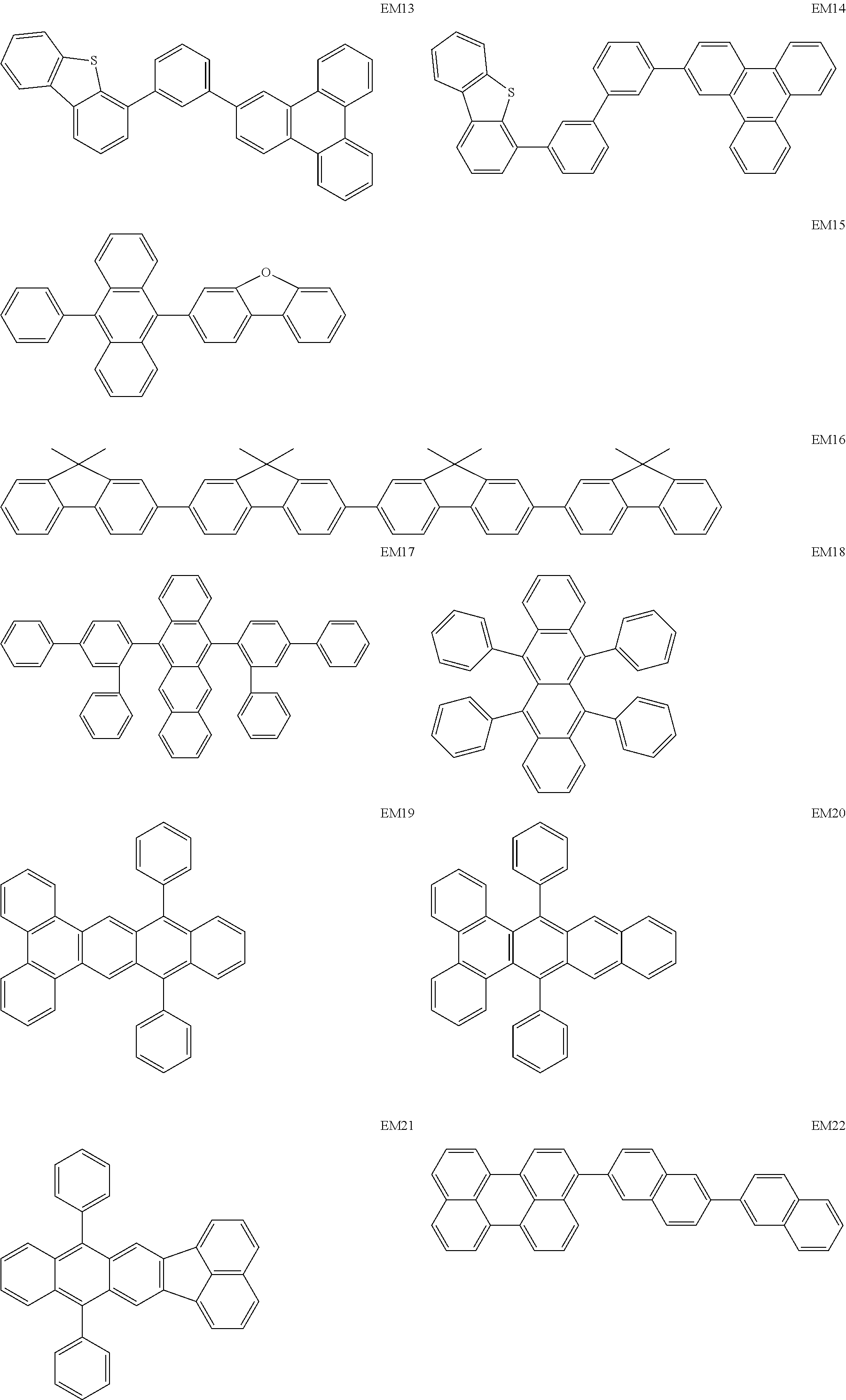

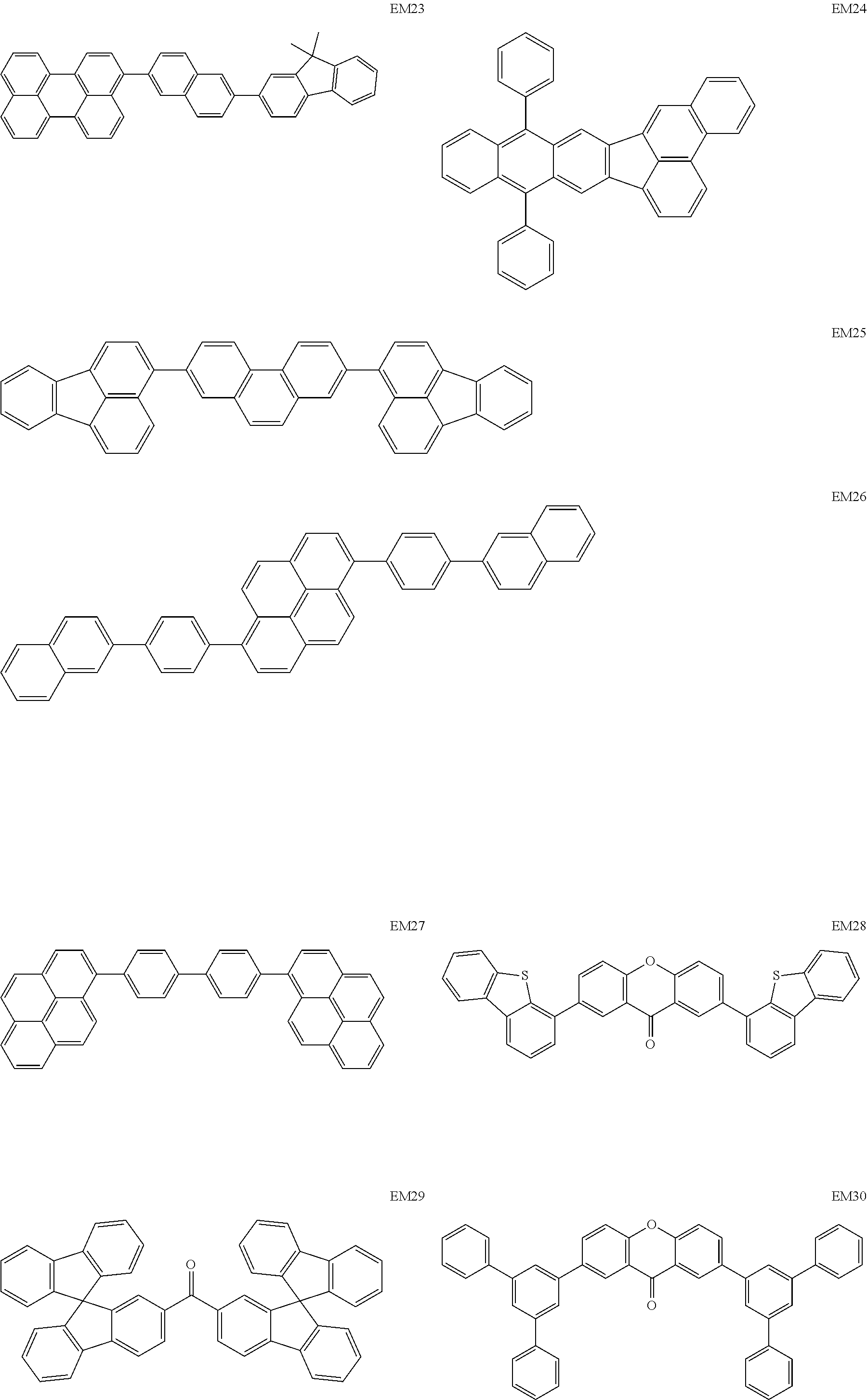

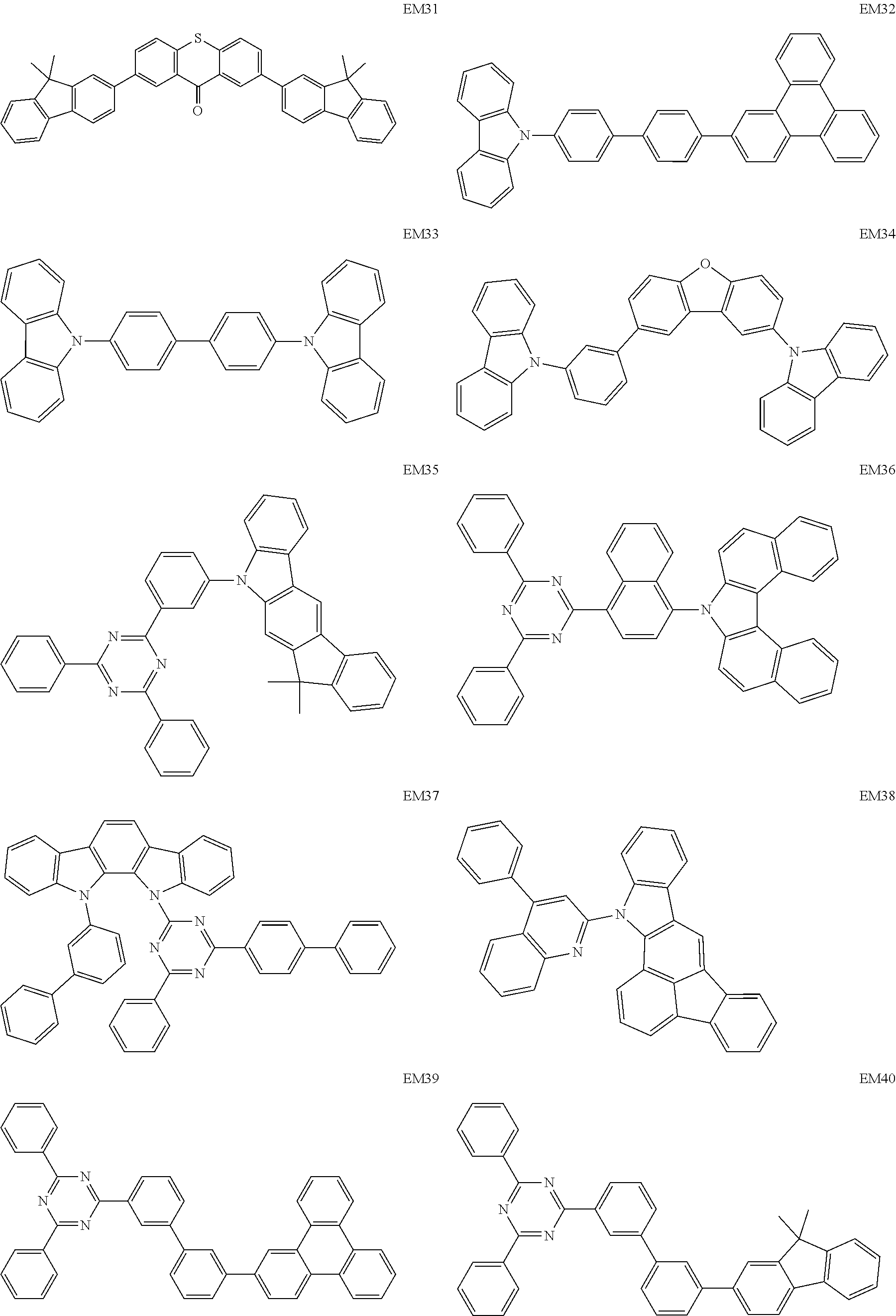

An electron-transport material can be selected from materials that can transport electrons injected from the negative electrode to the light-emitting layer and is selected in consideration of the balance with the hole mobility of a hole-transport material and the like. Examples of materials with electron-transport ability include, but are not limited to, oxadiazole derivatives, oxazole derivatives, pyrazine derivatives, triazole derivatives, triazine derivatives, quinoline derivatives, quinoxaline derivatives, phenanthroline derivatives, organoaluminum complexes, and fused-ring compounds (for example, fluorene derivatives, naphthalene derivatives, chrysene derivatives, and anthracene derivatives). Furthermore, the electron-transport material is also suitably used for a hole-blocking layer. Specific examples of compounds that can be used as electron-transport materials include, but are not limited to, the following.

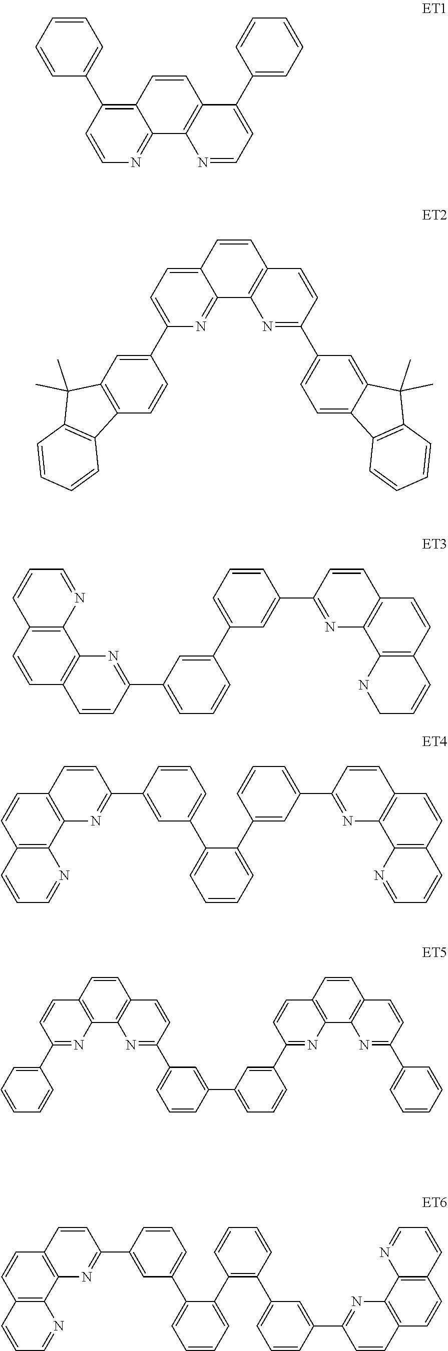

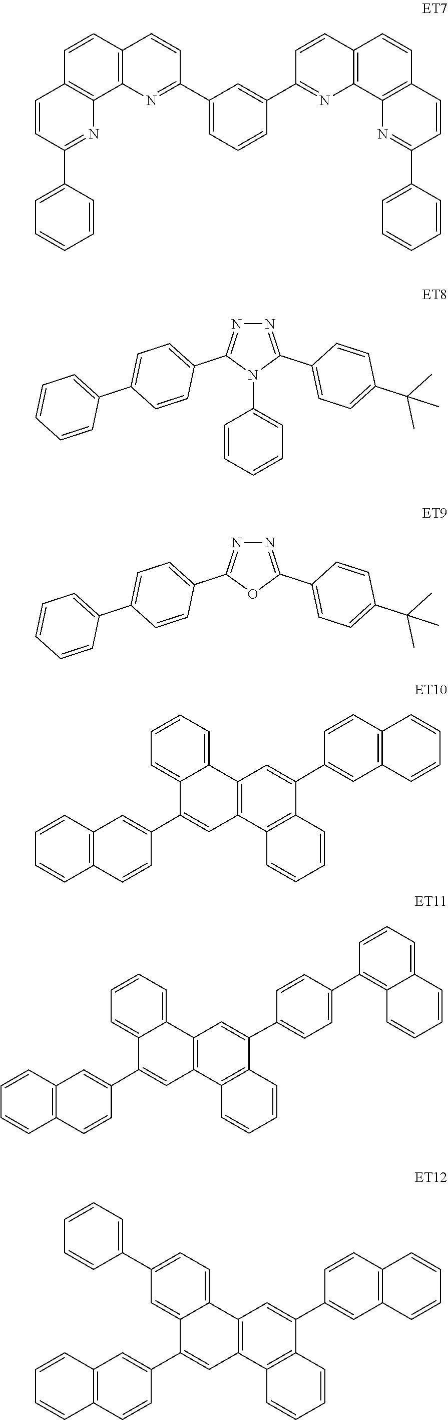

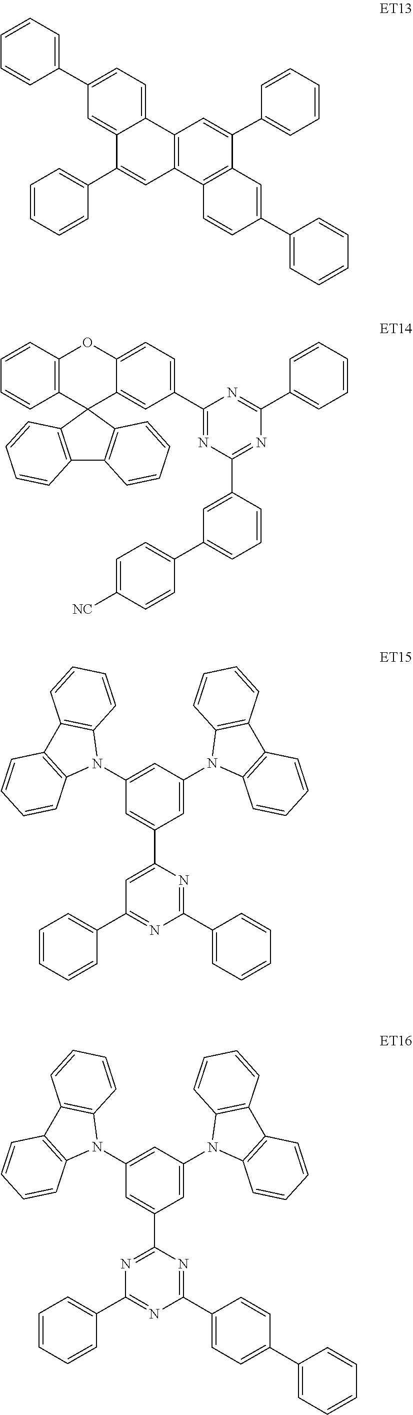

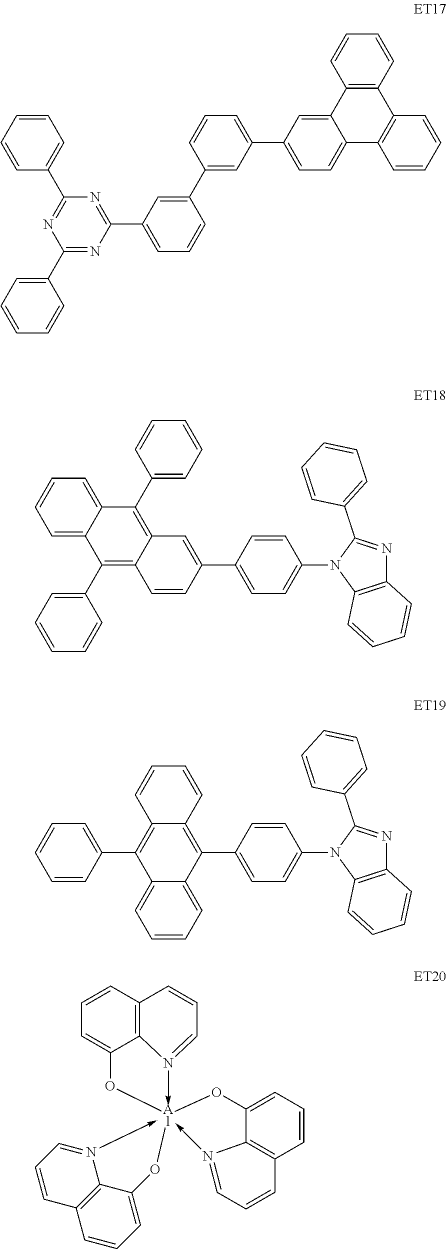

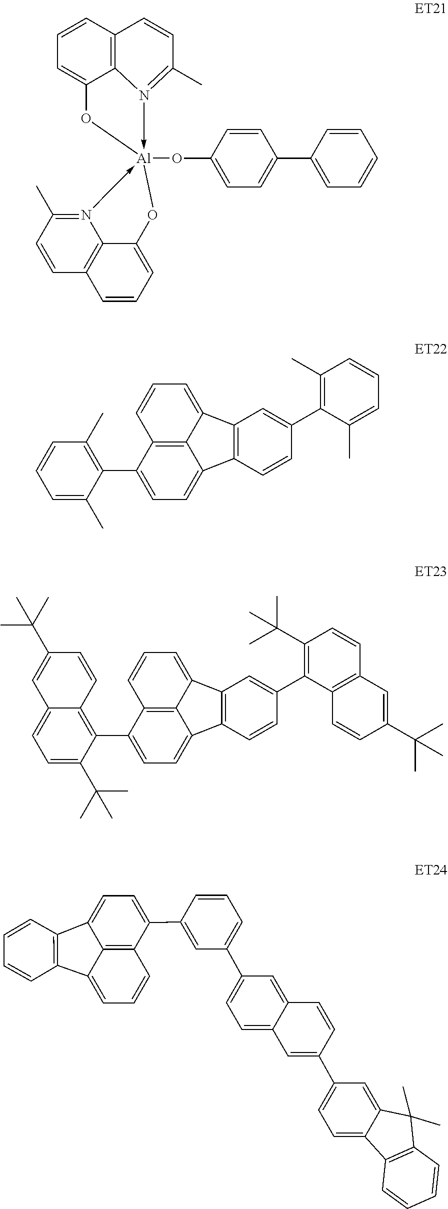

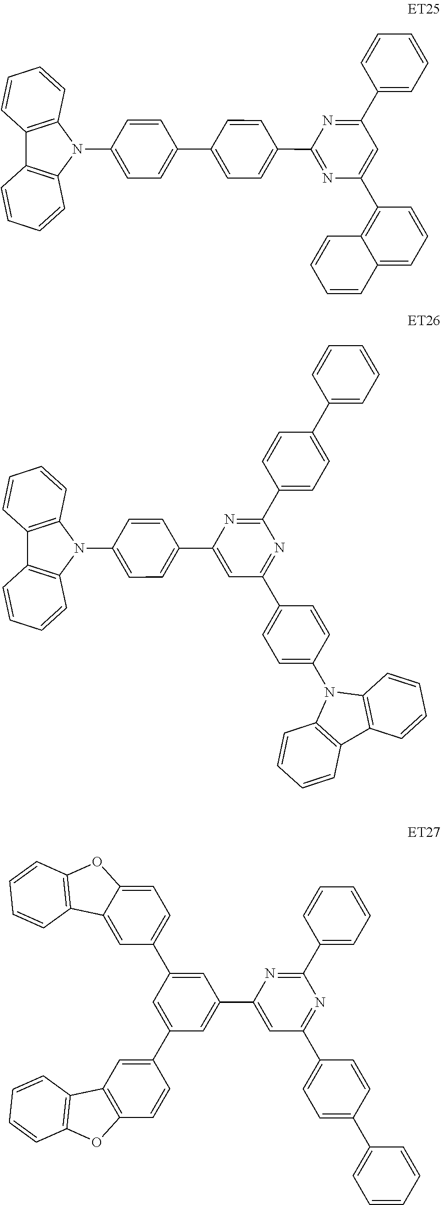 [SMILES: c1ccc(-c2cc(-c3cccc4ccccc34)nc(-c3ccc(-c4ccc(-n5c6ccccc6c6ccccc65)cc4)cc3)n2)cc1 ; c1ccc(-c2ccc(-c3nc(-c4ccc(-n5c6ccccc6c6ccccc65)cc4)cc(-c4ccc(-n5c6ccccc6c6ccccc65)cc4)n3)cc2)cc1 ; c1ccc(-c2ccc(-c3cc(-c4cc(-c5ccc6oc7ccccc7c6c5)cc(-c5ccc6oc7ccccc7c6c5)c4)nc(-c4ccccc4)n3)cc2)cc1]

The electron-injection material can be selected from materials that can easily inject electrons from the negative electrode and is selected in consideration of the balance with the hole injection properties and the like. Organic compounds include n-type dopants and reducing dopants. Examples include compounds containing an alkali metal, such as lithium fluoride, lithium complexes, such as lithium quinolinol, benzimidazolidene derivatives, imidazolidene derivatives, fulvalene derivatives, and acridine derivatives. It can also be used in combination with the electron-transport material.

### <Structure of Organic Light-Emitting Element>

An organic light-emitting element includes an insulating layer, a first electrode, an organic compound layer, and a second electrode on a substrate. A protective layer, a color filter, a microlens, or the like may be provided on the second electrode. When a color filter is provided, a planarization layer may be provided between the color filter and a protective layer. The planarization layer may be composed of an acrylic resin or the like. The same applies to a planarization layer provided between a color filter and a microlens.

### [Substrate]

The substrate may be formed of quartz, glass, a silicon wafer, resin, metal, or the like. The substrate may have a switching element, such as a transistor, and wiring, on which an insulating layer may be provided. The insulating layer may be composed of any material, provided that the insulating layer can have a contact hole for wiring between the insulating layer and the first electrode and is insulated from unconnected wires. For example, the insulating layer may be formed of a resin, such as polyimide, silicon oxide, or silicon nitride.

### [Electrodes]

A pair of electrodes can be used as electrodes. The pair of electrodes may be a positive electrode and a negative electrode. When an electric field is applied in a direction in which the organic light-emitting element emits light, an electrode with a high electric potential is a positive electrode, and the other electrode is a negative electrode. In other words, the electrode that supplies holes to the light-emitting layer is a positive electrode, and the electrode that supplies electrons is a negative electrode.

A constituent material of the positive electrode can have as large a work function as possible. Examples of the constituent material include metal elements, such as gold, platinum, silver, copper, nickel, palladium, cobalt, selenium, vanadium, and tungsten, mixtures thereof, alloys thereof, and metal oxides, such as tin oxide, zinc oxide, indium oxide, indium tin oxide (ITO), and indium zinc oxide. Electrically conductive polymers, such as polyaniline, polypyrrole, and polythiophene, may also be used.

These electrode materials may be used alone or in combination. The positive electrode may be composed of a single layer or a plurality of layers.

When used as a reflective electrode, for example, chromium, aluminum, silver, titanium, tungsten, molybdenum, an alloy thereof, or a laminate thereof can be used. These materials can also function as a reflective film that does not have a role as an electrode. When used as a transparent electrode, an oxide transparent conductive layer, such as indium tin oxide (ITO) or indium zinc oxide, can be used. However, the present disclosure is not limited thereto. The electrodes may be formed by photolithography.

A constituent material of the negative electrode can be a material with a small work function. For example, an alkali metal, such as lithium, an alkaline-earth metal, such as calcium, a metal element, such as aluminum, titanium, manganese, silver, lead, or chromium, or a mixture thereof may be used. An alloy of these metal elements may also be used. For example, magnesium-silver, aluminum-lithium, aluminum-magnesium, silver-copper, or zinc-silver may be used. A metal oxide, such as indium tin oxide (ITO), may also be used. These electrode materials may be used alone or in combination. The negative electrode may be composed of a single layer or a plurality of layers. In particular, silver can be used, and a silver alloy can be used to reduce the aggregation of silver. As long as the aggregation of silver can be reduced, the alloy may have any ratio. For example, the ratio of silver to another metal may be 1:1, 3:1, or the like.

The negative electrode may be, but is not limited to, an oxide conductive layer, such as ITO, for a top emission element or a reflective electrode, such as aluminum (A1), for a bottom emission element. The negative electrode may be formed by any method. A direct-current or alternating-current sputtering method can achieve good film coverage and easily decrease resistance.

### [Organic Compound Layer]

The organic compound layer may be formed of a single layer or a plurality of layers. Depending on their functions, the plurality of layers may be referred to as a hole-injection layer, a hole-transport layer, an electron-blocking layer, a light-emitting layer, a hole-blocking layer, an electron-transport layer, or an electron-injection layer. The organic compound layer is composed mainly of an organic compound and may contain an inorganic atom or an inorganic compound. For example, the compound layer may contain copper, lithium, magnesium, aluminum, iridium, platinum, molybdenum, zinc, or the like. The organic compound layer may be located between the first electrode and the second electrode and may be in contact with the first electrode and the second electrode.

An organic compound layer (a hole-injection layer, a hole-transport layer, an electron-blocking layer, a light-emitting layer, a hole-blocking layer, an electron-transport layer, an electron-injection layer, etc.) constituting an organic light-emitting element according to an embodiment of the present disclosure is formed by the following method.

An organic compound layer constituting an organic light-emitting element according to an embodiment of the present disclosure can be formed by a dry process, such as a vacuum evaporation method, an ionized deposition method, sputtering, or plasma. Instead of the dry process, a wet process may also be employed in which a layer is formed by a known coating method (for example, spin coating, dipping, a casting method, an LB method, an ink jet method, etc.) using an appropriate solvent.

A layer formed by a vacuum evaporation method, a solution coating method, or the like undergoes little crystallization or the like and has high temporal stability. When a film is formed by a coating method, the film may also be formed in combination with an appropriate binder resin.

Examples of the binder resin include, but are not limited to, polyvinylcarbazole resins, polycarbonate resins, polyester resins, ABS resins, acrylic resins, polyimide resins, phenolic resins, epoxy resins, silicone resins, and urea resins.

These binder resins may be used alone or in combination as a homopolymer or a copolymer. If necessary, an additive agent, such as a known plasticizer, oxidation inhibitor, and/or ultraviolet absorbent, may also be used.

### [Protective Layer]

A protective layer may be provided on the second electrode. For example, a glass sheet with a moisture absorbent may be attached to the second electrode to decrease the amount of water or the like entering the organic compound layer and to reduce the occurrence of display defects. In another embodiment, a passivation film of silicon nitride or the like may be provided on the second electrode to decrease the amount of water or the like entering the organic compound layer. For example, after the second electrode is formed, the negative electrode is transferred to another chamber without breaking the vacuum, and a silicon nitride film with a thickness of 2 μm may be formed as a protective layer by a chemical vapor deposition (CVD) method. The protective layer may be formed by the CVD method followed by an atomic layer deposition (ALD) method. A film formed by the ALD method may be formed of any material such as silicon nitride, silicon oxide, or aluminum oxide. Silicon nitride may be further deposited by the CVD method on the film formed by the ALD method. The film formed by the ALD method may have a smaller thickness than the film formed by the CVD method. More specifically, the thickness may be 50% or less or even 10% or less.

### [Color Filter]

A color filter may be provided on the protective layer. For example, a color filter that matches the size of the organic light-emitting element may be provided on another substrate and may be bonded to the substrate on which the organic light-emitting element is provided, or a color filter may be patterned on the protective layer by photolithography. The color filter may be composed of a polymer.

### [Planarization Layer]

A planarization layer may be provided between the color filter and the protective layer. The planarization layer is provided to reduce the roughness of the underlayer. The planarization layer is sometimes referred to as a material resin layer with any purpose. The planarization layer may be composed of an organic compound and can be composed of a high-molecular-weight compound, though it may be composed of a low-molecular-weight compound.

The planarization layer may be provided above and below the color filter, and the constituent materials thereof may be the same or different. Specific examples include polyvinylcarbazole resins, polycarbonate resins, polyester resins, ABS resins, acrylic resins, polyimide resins, phenolic resins, epoxy resins, silicone resins, and urea resins.

### [Microlens]

An organic light-emitting element or an organic light-emitting apparatus may include an optical member, such as a microlens, on the light output side. The microlens may be composed of an acrylic resin, an epoxy resin, or the like. The microlens may be used to increase the amount of light extracted from the organic light-emitting element or the organic light-emitting apparatus and control the direction of the extracted light. The microlens may have a hemispherical shape. For a hemispherical microlens, the vertex of the microlens is a contact point between the hemisphere and a tangent line parallel to the insulating layer among the tangent lines in contact with the hemisphere. The vertex of the microlens in a cross-sectional view can be determined in the same manner. More specifically, the vertex of the microlens in a cross-sectional view is a contact point between the semicircle of the microlens and a tangent line parallel to the insulating layer among the tangent lines in contact with the semicircle.

The midpoint of the microlens can also be defined. In a cross section of the microlens, a midpoint of a line segment from one end point to the other end point of the arc can be referred to as a midpoint of the microlens. A cross section in which the vertex and the midpoint are determined may be perpendicular to the insulating layer.

### [Opposite Substrate]

An opposite substrate may be provided on the planarization layer. The opposite substrate is so called because it faces the substrate. The opposite substrate may be composed of the same material as the substrate. When the substrate is a first substrate, the opposite substrate may be a second substrate.

### [Pixel Circuit]

An organic light-emitting apparatus including an organic light-emitting element may include a pixel circuit coupled to the organic light-emitting element. The pixel circuit may be of an active matrix type, which independently controls the light emission of a first light-emitting element and a second light-emitting element. The active-matrix circuit may be voltage programmed or current programmed. The drive circuit has a pixel circuit for each pixel. The pixel circuit may include a light-emitting element, a transistor for controlling the luminous brightness of the light-emitting element, a transistor for controlling light emission timing, a capacitor for holding the gate voltage of the transistor for controlling the luminous brightness, and a transistor for GND connection without through the light-emitting element.

A light-emitting apparatus includes a display region and a peripheral region around the display region. The display region includes the pixel circuit, and the peripheral region includes a display control circuit. The mobility of a transistor constituting the pixel circuit may be smaller than the mobility of a transistor constituting the display control circuit. The gradient of the current-voltage characteristics of a transistor constituting the pixel circuit may be smaller than the gradient of the current-voltage characteristics of a transistor constituting the display control circuit. The gradient of the current-voltage characteristics can be determined by so-called Vg-Ig characteristics. A transistor constituting the pixel circuit is a transistor coupled to a light-emitting element, such as a first light-emitting element.

### [Pixel]

An organic light-emitting apparatus including an organic light-emitting element may have a plurality of pixels. Each pixel has subpixels that emit light of different colors. For example, the subpixels may have RGB emission colors.

In each pixel, a region also referred to as a pixel aperture emits light. This region is the same as the first region. The pixel aperture may be 15 μm or less or 5 μm or more. More specifically, the pixel aperture may be 11 μm, 9.5 μm, 7.4 μm, or 6.4 μm. The distance between the subpixels may be 10 μm or less, more specifically, 8 μm, 7.4 μm, or 6.4 μm.

The pixels may be arranged in a known form in a plan view. Examples include a stripe arrangement, a delta arrangement, a PenTile arrangement, and a Bayer arrangement. Each subpixel may have any known shape in a plan view. Examples include quadrangles, such as a rectangle and a rhombus, and a hexagon. As a matter of course, the rectangle also includes a figure that is not strictly rectangular but is close to rectangular. The shape of each subpixel and the pixel array can be used in combination.

### <Applications of Organic Light-Emitting Element>

The organic light-emitting element according to the present embodiment can be used as a constituent of a display apparatus or a lighting apparatus. Other applications include an exposure light source for an electrophotographic image-forming apparatus, a backlight for a liquid crystal display, and a light-emitting apparatus with a color filter in a white light source.

The display apparatus may be an image-information-processing apparatus that includes an image input unit for inputting image information from an area CCD, a linear CCD, a memory card, or the like, includes an information processing unit for processing the input information, and displays an input image on a display unit. The display apparatus may have a plurality of pixels, and at least one of the pixels may include the organic light-emitting element according to the present embodiment and a transistor coupled to the organic light-emitting element.

A display unit of an imaging apparatus or an ink jet printer may have a touch panel function. A driving system of the touch panel function may be, but is not limited to, an infrared radiation system, an electrostatic capacitance system, a resistive film system, or an electromagnetic induction system. The display apparatus may be used for a display unit of a multifunction printer.

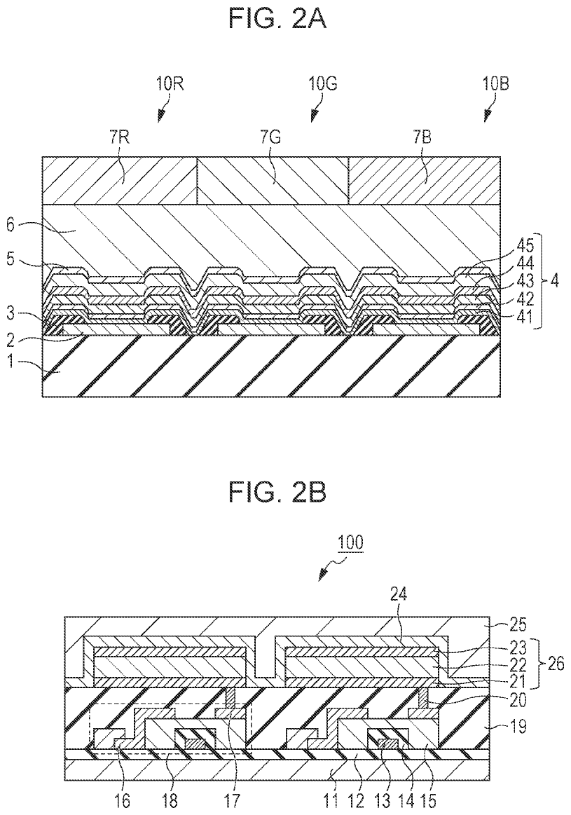

Next, a display apparatus according to the present embodiment is described with reference to the accompanying drawings. FIGS. 2A and 2B are schematic cross-sectional views of an example of a display apparatus that includes an organic light-emitting element and a transistor coupled to the organic light-emitting element. The transistor is an example of an active element. The transistor may be a thin-film transistor (TFT).

FIG. 2A illustrates an example of a pixel serving as a constituent of the display apparatus according to the present embodiment. The pixel has subpixels 10. The subpixels are 10R, 10G, and 10B with different emission colors. The emission colors may be distinguished by the wavelength of light emitted from the light-emitting layer, or light emitted from each subpixel may be selectively transmitted or color-converted with a color filter or the like. Each of the subpixels 10 includes a reflective electrode serving as a first electrode 2, an insulating layer 3 covering an end of the first electrode 2, organic compound layers 4 covering the first electrode 2 and the insulating layer 3, a transparent electrode serving as a second electrode 5, a protective layer 6, and a color filter 7 on an interlayer insulating layer 1.

A transistor and/or a capacitor element may be provided under or inside the interlayer insulating layer 1. The transistor and the first electrode 2 may be electrically connected via a contact hole (not shown) or the like.

The insulating layer 3 is also referred to as a bank or a pixel separation film. The insulating layer 3 covers the ends of the first electrode 2 and surrounds the first electrode 2. A portion not covered with the insulating layer 3 is in contact with the organic compound layers 4 and serves as a light-emitting region.

The organic compound layers 4 include a hole-injection layer 41, a hole-transport layer 42, a first light-emitting layer 43, a second light-emitting layer 44, and an electron-transport layer 45.

The second electrode 5 may be a transparent electrode, a reflective electrode, or a semitransparent electrode.

The protective layer 6 reduces the penetration of moisture into the organic compound layers 4. The protective layer 6 is illustrated as a single layer but may be a plurality of layers. The protective layer may include an inorganic compound layer and an organic compound layer.

The color filter 7 is divided into 7R, 7G, and 7B according to the color. The color filter 7 may be formed on a planarization film (not shown). Furthermore, a resin protective layer (not shown) may be provided on the color filter 7. The color filter 7 may be formed on the protective layer 6. Alternatively, the color filter 7 may be bonded after being provided on an opposite substrate, such as a glass substrate.

A display apparatus 100 in FIG. 2B includes an organic light-emitting element 26 and a TFT 18 as an example of a transistor. The display apparatus 100 includes a substrate 11 made of glass, silicon, or the like and an insulating layer 12 on the substrate 11. An active element, such as the TFT 18, and a gate electrode 13, a gate-insulating film 14, and a semiconductor layer 15 of the active element are provided on the insulating layer 12. The TFT 18 is also composed of a drain electrode 16 and a source electrode 17. The TFT 18 is covered with an insulating film 19. A positive electrode 21 of the organic light-emitting element 26 is coupled to the source electrode 17 through a contact hole 20 formed in the insulating film 19.

Electrical connection between the electrodes of the organic light-emitting element 26 (the positive electrode 21 and a negative electrode 23) and the electrodes of the TFT 18 (the source electrode 17 and the drain electrode 16) is not limited to that illustrated in FIG. 2B. More specifically, it is only necessary to electrically connect either the positive electrode 21 or the negative electrode 23 to either the source electrode 17 or the drain electrode 16 of the TFT 18. TFT refers to a thin-film transistor.

Although an organic compound layer 22 is a single layer in the display apparatus 100 illustrated in FIG. 2B, the organic compound layer 22 may be composed of a plurality of layers. The negative electrode 23 is covered with a first protective layer 24 and a second protective layer 25 for preventing degradation of the organic light-emitting element 26.

Although the display apparatus 100 illustrated in FIG. 2B includes a transistor as a switching element, another element may be used instead as the switching element.

The transistor used in the display apparatus 100 in FIG. 2B is not limited to a transistor including a single crystal silicon wafer and may also be a thin-film transistor including an active layer on an insulating surface of a substrate. The active layer may be single-crystal silicon, non-single-crystal silicon, such as amorphous silicon or microcrystalline silicon, or a non-single-crystal oxide semiconductor, such as indium zinc oxide or indium gallium zinc oxide. The thin-film transistor is also referred to as a TFT element.

The transistor in the display apparatus 100 illustrated in FIG. 2B may be formed within a substrate, such as a Si substrate. The phrase “formed within a substrate” means that the substrate, such as a Si substrate, itself is processed to form the transistor. Thus, the transistor within the substrate can be considered that the substrate and the transistor are integrally formed.

In the organic light-emitting element according to the present embodiment, the luminous brightness is controlled with the TFT, which is an example of a switching element. The organic light-emitting element can be provided in a plurality of planes to display an image at each luminous brightness. The switching element according to the present embodiment is not limited to the TFT and may be a transistor formed of low-temperature polysilicon or an active-matrix driver formed on a substrate, such as a Si substrate. “On a substrate” may also be referred to as “within a substrate”. Whether a transistor is provided within a substrate or a TFT is used depends on the size of a display unit. For example, for an approximately 0.5-inch display unit, an organic light-emitting element can be provided on a Si substrate.

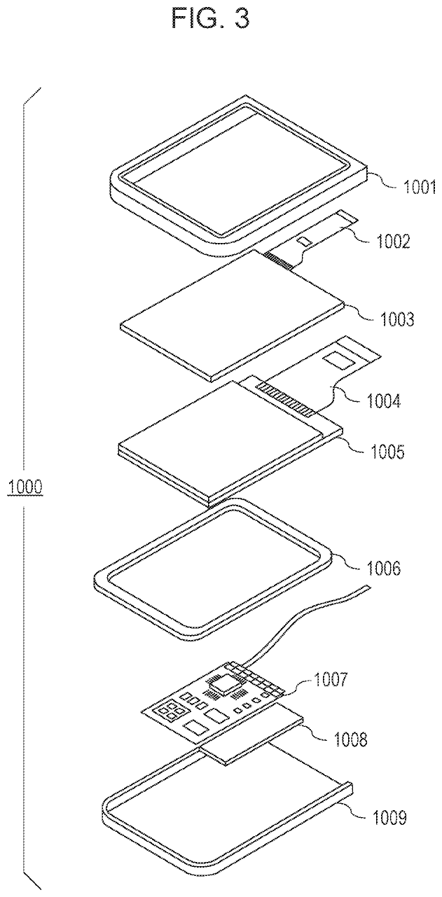

FIG. 3 is a schematic view of an example of the display apparatus according to the present embodiment. A display apparatus 1000 may include a touch panel 1003, a display panel 1005, a frame 1006, a circuit substrate 1007, and a battery 1008 between an upper cover 1001 and a lower cover 1009. The touch panel 1003 and the display panel 1005 are coupled to flexible print circuits FPC 1002 and 1004, respectively. Transistors are printed on the circuit substrate 1007. The battery 1008 may not be provided when the display apparatus is not a mobile device, or may be provided at another position even when the display apparatus is a mobile device.

The display apparatus according to the present embodiment may include color filters of red, green, and blue colors. In the color filters, the red, green, and blue colors may be arranged in a delta arrangement.

The display apparatus according to the present embodiment may be used for a display unit of a mobile terminal. Such a display apparatus may have both a display function and an operation function. Examples of the mobile terminal include mobile phones, such as smartphones, tablets, and head-mounted displays.

The display apparatus according to the present embodiment may be used for a display unit of an imaging apparatus that includes an optical unit with a plurality of lenses and an imaging element for receiving light passing through the optical unit. The imaging apparatus may include a display unit for displaying information acquired by the imaging element. The display unit may be a display unit exposed outside from the imaging apparatus or a display unit located in a finder. The imaging apparatus may be a digital camera or a digital video camera.

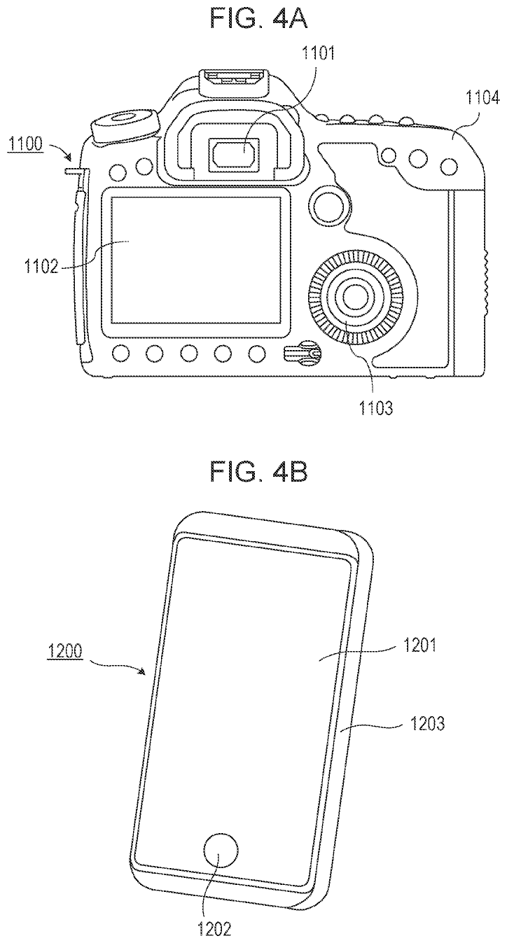

FIG. 4A is a schematic view of an example of an imaging apparatus according to the present embodiment. An imaging apparatus 1100 may include a viewfinder 1101, a rear display 1102, an operating unit 1103, and a housing 1104. The viewfinder 1101 may include the display apparatus according to the present embodiment. In such a case, the display apparatus may display environmental information, imaging instructions, and the like as well as an image to be captured. The environmental information may include the intensity of external light, the direction of external light, the travel speed of the photographic subject, the possibility that the photographic subject is shielded by a shielding material, and the like.

Because the appropriate timing for imaging is a short time, it is better to display information as soon as possible. Thus, a display apparatus including the organic light-emitting element according to the present embodiment can be used. This is because the organic light-emitting element has a high response speed. A display apparatus including the organic light-emitting element can be more suitably used than these apparatuses and liquid crystal displays that require a high display speed.

The imaging apparatus 1100 includes an optical unit (not shown). The optical unit has a plurality of lenses and focuses an image on an imaging element in the housing 1104. The focus of the lenses can be adjusted by adjusting their relative positions. This operation can also be automatically performed. The imaging apparatus may also be referred to as a photoelectric conversion apparatus. The photoelectric conversion apparatus can have, as an imaging method, a method of detecting a difference from a previous image or a method of cutting out a permanently recorded image, instead of taking an image one after another.

FIG. 4B is a schematic view of an example of an electronic equipment according to the present embodiment. Electronic equipment 1200 includes a display unit 1201, an operating unit 1202, and a housing 1203. The housing 1203 may include a circuit, a printed circuit board including the circuit, a battery, and a communication unit. The operating unit 1202 may be a button or a touch panel response unit. The operating unit 1202 may be a biometric recognition unit that recognizes a fingerprint and releases the lock. Electronic equipment with a communication unit may also be referred to as communication equipment. The electronic equipment 1200 may have a lens and an imaging element and thereby further have a camera function. An image captured by the camera function is displayed on the display unit 1201. The electronic equipment 1200 may be a smartphone, a notebook computer, or the like.

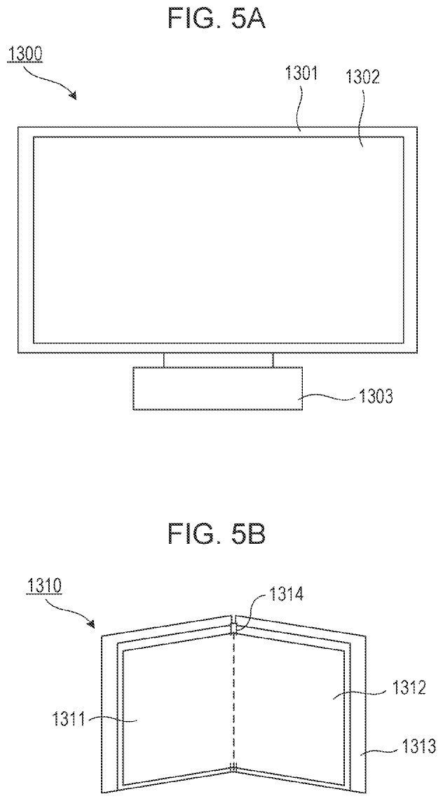

FIGS. 5A and 5B are schematic views of an example of the display apparatus according to the present embodiment. FIG. 5A illustrates a display apparatus, such as a television monitor or a PC monitor. A display apparatus 1300 includes a frame 1301 and a display unit 1302. The light-emitting element according to the present embodiment may be used for the display unit 1302. The frame 1301 and the display unit 1302 are supported by a base 1303. The base 1303 is not limited to the structure illustrated in FIG. 5A. The lower side of the frame 1301 may also serve as the base. The frame 1301 and the display unit 1302 may be bent. The radius of curvature may be 5000 mm or more and 6000 mm or less.

FIG. 5B is a schematic view of another example of the display apparatus according to the present embodiment. A display apparatus 1310 in FIG. 5B is configured to be foldable and is a so-called foldable display apparatus. The display apparatus 1310 includes a first display unit 1311, a second display unit 1312, a housing 1313, and a folding point 1314. The first display unit 1311 and the second display unit 1312 may include the light-emitting element according to the present embodiment. The first display unit 1311 and the second display unit 1312 may be a single display apparatus without a joint. The first display unit 1311 and the second display unit 1312 can be divided by the folding point. The first display unit 1311 and the second display unit 1312 may display different images or one image.

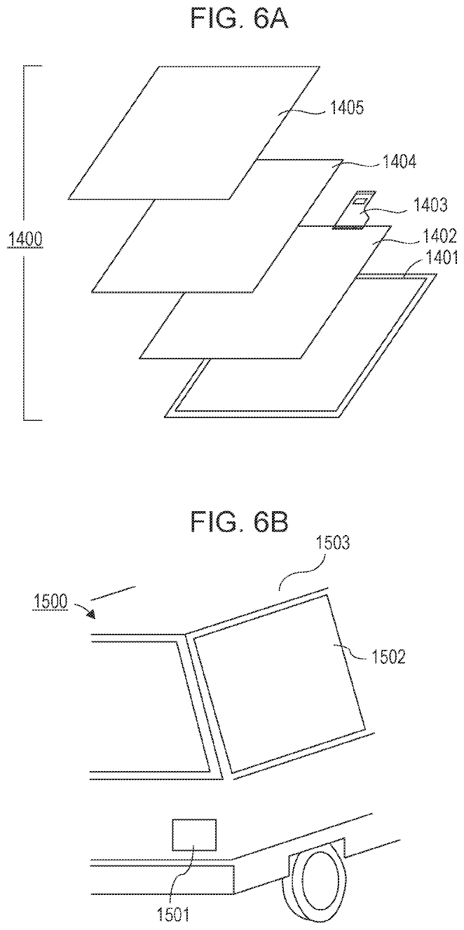

FIG. 6A is a schematic view of an example of a lighting apparatus according to the present embodiment. A lighting apparatus 1400 may include a housing 1401, a light source 1402, a circuit substrate 1403, an optical filter 1404 that transmits light emitted by the light source 1402, and a light-diffusing unit 1405. The light source 1402 may include the organic light-emitting element according to the present embodiment. The optical filter 1404 may be a filter that improves the color rendering properties of the light source. The light-diffusing unit 1405 can effectively diffuse light from the light source and widely spread light as in illumination. The optical filter 1404 and the light-diffusing unit 1405 may be provided on the light output side of the illumination. If necessary, a cover may be provided on the outermost side.

For example, the lighting apparatus is an interior lighting apparatus. The lighting apparatus may emit white light, neutral white light, or light of any color from blue to red. The lighting apparatus may have a light control circuit for controlling such light. The lighting apparatus may include the organic light-emitting element according to the present embodiment and a power supply circuit coupled thereto. The power supply circuit is a circuit that converts an AC voltage to a DC voltage. White has a color temperature of 4200 K, and neutral white has a color temperature of 5000 K. The lighting apparatus may have a color filter.

The lighting apparatus according to the present embodiment may include a heat dissipation unit. The heat dissipation unit releases heat from the apparatus to the outside and may be a metal or liquid silicon with a high specific heat.

FIG. 6B is a schematic view of an automobile as an example of a moving body according to the present embodiment. The automobile has a taillight as an example of a lamp. An automobile 1500 may have a taillight 1501, which comes on when a brake operation or the like is performed.

The taillight 1501 may include the organic light-emitting element according to the present embodiment. The taillight 1501 may include a protective member for protecting the organic light-emitting element. The protective member may be formed of any transparent material with moderately high strength and can be formed of polycarbonate or the like. The polycarbonate may be mixed with a furan dicarboxylic acid derivative, an acrylonitrile derivative, or the like.

The automobile 1500 may have a body 1503 and a window 1502 on the body 1503. The window 1502 may be a transparent display as long as it is not a window for checking the front and rear of the automobile. The transparent display may include the organic light-emitting element according to the present embodiment. In such a case, constituent materials, such as electrodes, of the organic light-emitting element are transparent materials.

The moving body according to the present embodiment may be a ship, an aircraft, a drone, or the like. The moving body may include a body and a lamp provided on the body. The lamp may emit light to indicate the position of the body. The lamp includes the organic light-emitting element according to the present embodiment.

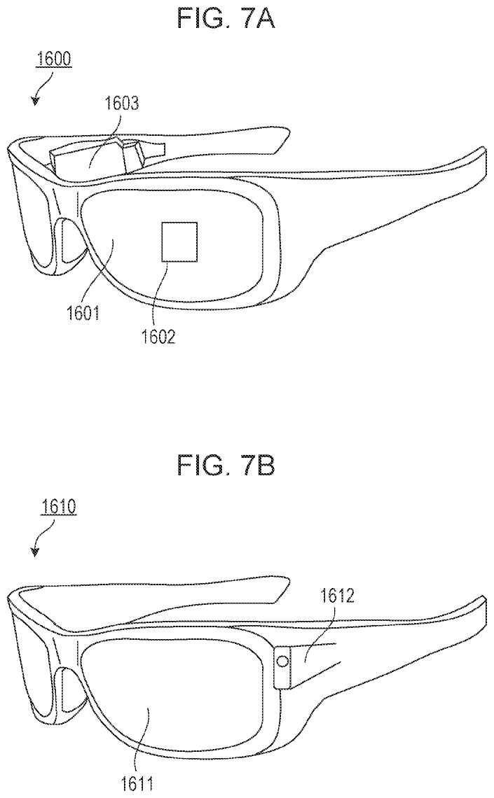

Application examples of the display apparatus according to one of the embodiments are described below with reference to FIGS. 7A and 7B. The display apparatus can be applied to a system that can be worn as a wearable device, such as smart glasses, a head-mounted display (HMD), or smart contact lenses. An imaging and displaying apparatus used in such an application example includes an imaging apparatus that can photoelectrically convert visible light and a display apparatus that can emit visible light.

FIG. 7A is a schematic view of an example of a wearable device according to an embodiment of the present disclosure. FIG. 7A illustrates glasses 1600 (smart glasses) according to one application example. An imaging apparatus 1602, such as a complementary metal-oxide semiconductor (CMOS) sensor or a single-photon avalanche photodiode (SPAD), is provided on the front side of a lens 1601 of the glasses 1600. The display apparatus according to one of the embodiments is provided on the back side of the lens 1601.

The glasses 1600 further include a controller 1603. The controller 1603 functions as a power supply for supplying power to the imaging apparatus 1602 and the display apparatus. The controller 1603 controls the operation of the imaging apparatus 1602 and the display apparatus. The lens 1601 has an optical system for focusing light on the imaging apparatus 1602.

FIG. 7B is a schematic view of another example of a wearable device according to an embodiment of the present disclosure. FIG. 7B illustrates glasses 1610 (smart glasses) according to one application example. The glasses 1610 have a controller 1612, which includes an imaging apparatus corresponding to the imaging apparatus 1602 of FIG. 7A and a display apparatus. A lens 1611 includes an optical system for projecting light from the imaging apparatus of the controller 1612 and the display apparatus, and an image is projected on the lens 1611. The controller 1612 functions as a power supply for supplying power to the imaging apparatus and the display apparatus and controls the operation of the imaging apparatus and the display apparatus.

The controller 1612 may include a line-of-sight detection unit for detecting the line of sight of the wearer. Infrared radiation may be used to detect the line of sight. An infrared radiation unit emits infrared light to an eyeball of a user who is gazing at a display image. Reflected infrared light from the eyeball is detected by an imaging unit including a light-receiving element to capture an image of the eyeball. A reduction unit for reducing light from the infrared radiation unit to a display unit in a plan view is provided to reduce degradation in image quality. The line of sight of the user for the display image is detected from the image of the eyeball captured by infrared imaging. Any known technique can be applied to line-of-sight detection using the image of the eyeball. For example, it is possible to use a line-of-sight detection method based on a Purkinje image obtained by the reflection of irradiation light by the cornea. More specifically, a line-of-sight detection process based on a pupil-corneal reflection method is performed. The line of sight of the user is detected by calculating a line-of-sight vector representing the direction (rotation angle) of an eyeball on the basis of an image of a pupil and a Purkinje image included in a captured image of the eyeball using the pupil-corneal reflection method.

A display apparatus according to an embodiment of the present disclosure may include an imaging apparatus including a light-receiving element and may control a display image on the basis of line-of-sight information of a user from the imaging apparatus. More specifically, on the basis of the line-of-sight information, the display apparatus determines a first visibility region at which the user gazes and a second visibility region other than the first visibility region. The first visibility region and the second visibility region may be determined by the controller of the display apparatus or may be received from an external controller. In the display region of the display apparatus, the first visibility region may be controlled to have higher display resolution than the second visibility region. In other words, the second visibility region may have lower resolution than the first visibility region.

The display region has a first display region and a second display region different from the first display region, and the priority of the first display region and the second display region depends on the line-of-sight information. The first visibility region and the second visibility region may be determined by the controller of the display apparatus or may be received from an external controller. A region with a higher priority may be controlled to have higher resolution than another region. In other words, a region with a lower priority may have lower resolution.

The first visibility region or a region with a higher priority may be determined by artificial intelligence (AI). The AI may be a model configured to estimate the angle of the line of sight and the distance to a target ahead of the line of sight from an image of an eyeball using the image of the eyeball and the direction in which the eyeball actually viewed in the image as teaching data. The AI program may be stored in the display apparatus, the imaging apparatus, or an external device. The AI program stored in an external device is transmitted to the display apparatus via communication.

For display control based on visual recognition detection, the present disclosure can be applied to smart glasses further having an imaging apparatus for imaging the outside. Smart glasses can display captured external information in real time.

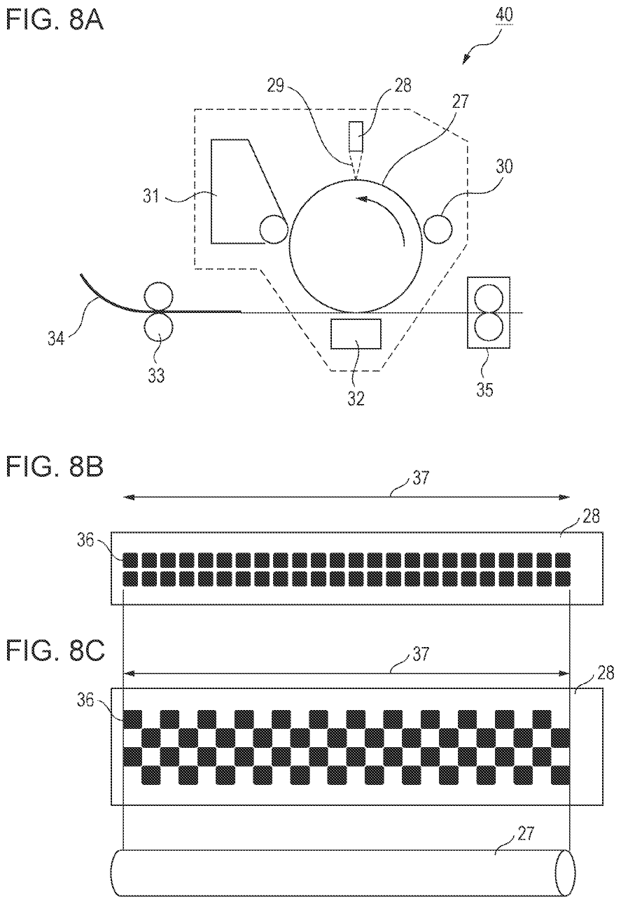

FIG. 8A is a schematic view of an example of an image-forming apparatus according to an embodiment of the present disclosure. An image-forming apparatus 40 is an electrophotographic image-forming apparatus and includes a photosensitive member 27, an exposure light source 28, a charging unit 30, a developing unit 31, a transfer unit 32, a conveying roller 33, and a fixing unit 35. The exposure light source 28 emits light 29, and an electrostatic latent image is formed on the surface of the photosensitive member 27. The exposure light source 28 includes the organic light-emitting element according to the present embodiment. The developing unit 31 contains toner and the like. The charging unit 30 electrifies the photosensitive member 27. The transfer unit 32 transfers a developed image onto a recording medium 34. The conveying roller 33 conveys the recording medium 34. The recording medium 34 is paper, for example. The fixing unit 35 fixes an image on the recording medium 34.

FIGS. 8B and 8C are schematic views of the exposure light source 28 in which a plurality of light-emitting portions 36 are arranged on a long substrate. An arrow 37 indicates a direction parallel to the axis of the photosensitive member and indicates a column direction in which the organic light-emitting elements are arranged. This column direction is the same as the direction of the rotation axis of the photosensitive member 27. This direction can also be referred to as the major-axis direction of the photosensitive member 27. In FIG. 8B, the light-emitting portions 36 are arranged in the major-axis direction of the photosensitive member 27. In FIG. 8C, unlike FIG. 8B, the light-emitting portions 36 are alternately arranged in the column direction in the first and second columns. The first and second columns are arranged at different positions in the row direction. In the first column, the light-emitting portions 36 are arranged at intervals. In the second column, the light-emitting portions 36 are arranged at positions corresponding to the spaces between the light-emitting portions 36 of the first column. Thus, the light-emitting portions 36 are also arranged at intervals in the row direction. The arrangement in FIG. 8C can also be referred to as a grid-like pattern, a staggered pattern, or a checkered pattern, for example.

As described above, an apparatus including the organic light-emitting element according to the present embodiment can be used to stably display a high-quality image for extended periods.

### EXEMPLARY EMBODIMENTS

The present disclosure is described below with exemplary embodiments. However, the present disclosure is not limited these exemplary embodiments.

### Exemplary Embodiment 1 (Synthesis of Exemplary Compound A1)

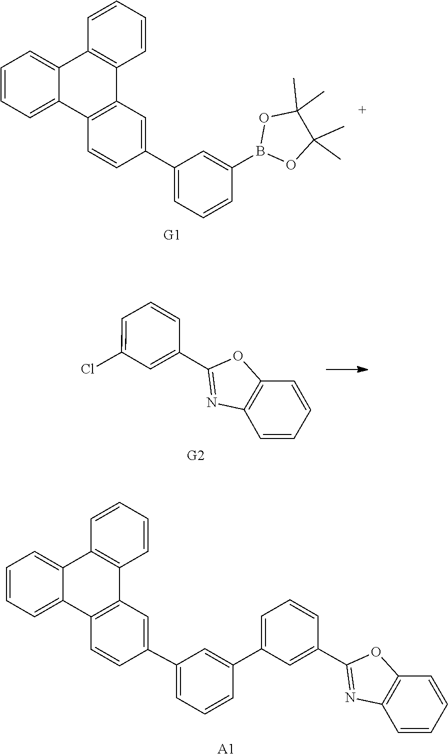

A 200-ml recovery flask was charged with the following reagents and solvents.
Compound G1: 2.0 g (4.6 mmol)
Compound G2: 1.2 g (5.1 mmol)
Pd(dba)₂: 0.80 g
sphos: 0.19 g
Potassium phosphate: 2.0 g
Toluene: 50 ml
H₂O: 5 ml

The reaction solution was then heated and stirred under reflux in a nitrogen stream for 6 hours. After completion of the reaction, water was added to the product for separation. The product was dissolved in chloroform, was purified by column chromatography (chloroform:heptane), and was recrystallized in toluene/heptane to give 1.5 g (yield: 65%) of an exemplary compound A1 as a white solid.

The exemplary compound A1 was subjected to mass spectrometry with MALDI-TOF-MS (Autoflex LRF manufactured by Bruker).

### [MALDI-TOF-MS]

Measured value: m/z=498 Calculated value: C₃₇H₂₃NO=498

### Exemplary Embodiments 2 to 23 (Synthesis of Exemplary Compounds), Synthesis of Comparative Compounds

Compounds were synthesized in the same manner as in Exemplary Embodiment 1 except that the compounds shown in Tables 5-1 to 9 were used as the raw materials G1 and G2. Actual values m/z measured by mass spectrometry in the same manner as in Exemplary Embodiment 1 are also shown.

TABLE 5-1

| Exemplary | Exemplary | | | |
|---|---|---|---|---|
| embodiment | compound | Raw material G1 | Raw material G2 | m/z |
| 1 | A1 | 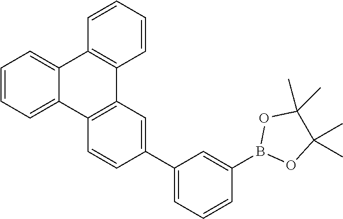 [SMILES: CC1(C)OB(c2cccc(-c3ccc4c5ccccc5c5ccccc5c4c3)c2)OC1(C)C] | 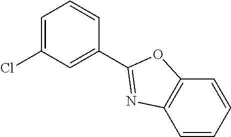 [SMILES: Clc1cccc(-c2nc3ccccc3o2)c1] | 498 |
| 2 | A4 | 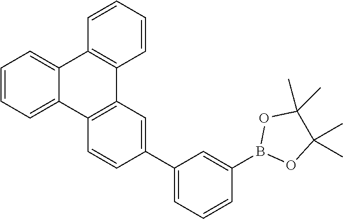 [SMILES: CC1(C)OB(c2cccc(-c3ccc4c5ccccc5c5ccccc5c4c3)c2)OC1(C)C] | 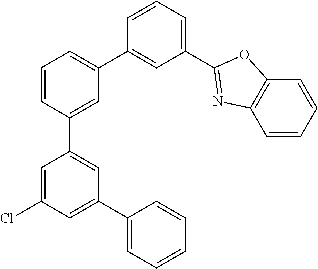 [SMILES: Clc1cc(-c2ccccc2)cc(-c2cccc(-c3cccc(-c4nc5ccccc5o4)c3)c2)c1] | 726 |
| 3 | A13 | 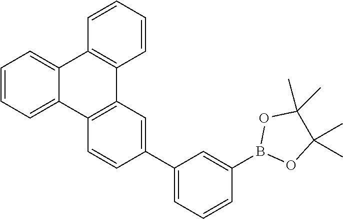 [SMILES: CC1(C)OB(c2cccc(-c3ccc4c5ccccc5c5ccccc5c4c3)c2)OC1(C)C] | 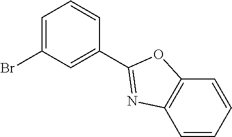 [SMILES: Brc1cccc(-c2nc3ccccc3o2)c1] | 514 |
| 4 | A14 | 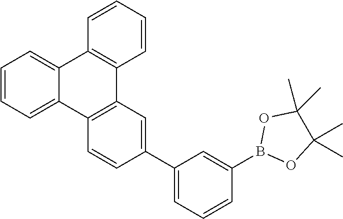 [SMILES: CC1(C)OB(c2cccc(-c3ccc4c5ccccc5c5ccccc5c4c3)c2)OC1(C)C] | 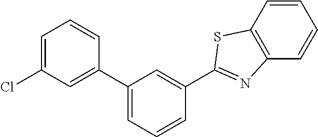 [SMILES: Clc1cccc(-c2cccc(-c3nc4ccccc4s3)c2)c1] | 590 |
| 5 | A15 | 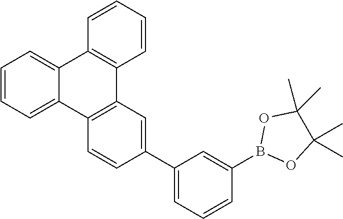 [SMILES: CC1(C)OB(c2cccc(-c3ccc4c5ccccc5c5ccccc5c4c3)c2)OC1(C)C] | 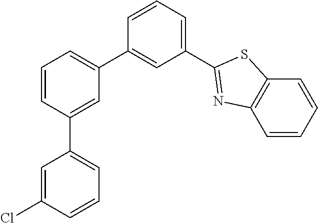 [SMILES: Clc1cccc(-c2cccc(-c3cccc(-c4nc5ccccc5s4)c3)c2)c1] | 666 |
| 6 | A16 | 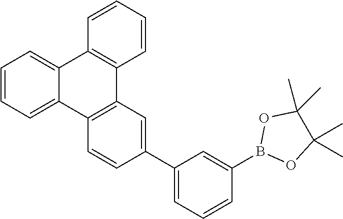 [SMILES: CC1(C)OB(c2cccc(-c3ccc4c5ccccc5c5ccccc5c4c3)c2)OC1(C)C] | 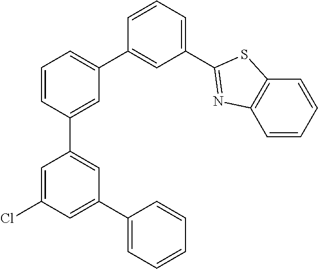 [SMILES: Clc1cc(-c2ccccc2)cc(-c2cccc(-c3cccc(-c4nc5ccccc5s4)c3)c2)c1] | 742 |
| 7 | A20 | 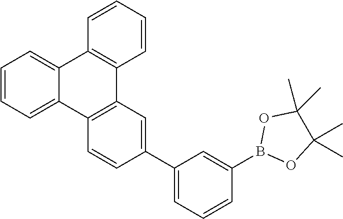 [SMILES: CC1(C)OB(c2cccc(-c3ccc4c5ccccc5c5ccccc5c4c3)c2)OC1(C)C] | 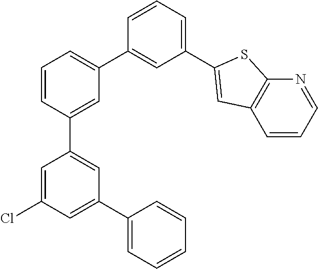 [SMILES: Clc1cc(-c2ccccc2)cc(-c2cccc(-c3cccc(-c4cc5cccnc5s4)c3)c2)c1] | 742 |
| 8 | A24 | 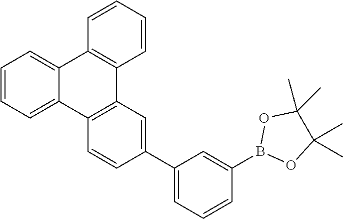 [SMILES: CC1(C)OB(c2cccc(-c3ccc4c5ccccc5c5ccccc5c4c3)c2)OC1(C)C] | 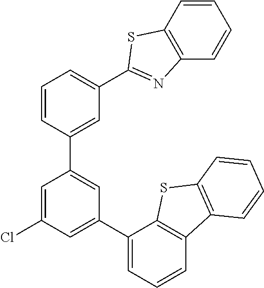 [SMILES: Clc1cc(-c2cccc(-c3nc4ccccc4s3)c2)cc(-c2cccc3c2sc2ccccc23)c1] | 799 |
| 9 | B1 | 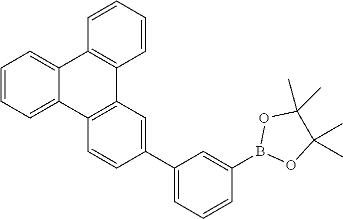 [SMILES: CC1(C)OB(c2cccc(-c3ccc4c5ccccc5c5ccccc5c4c3)c2)OC1(C)C] | 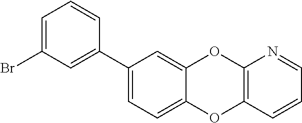 [SMILES: Brc1cccc(-c2ccc3c(c2)Oc2ncccc2O3)c1] | 564 |
| 10 | B2 | 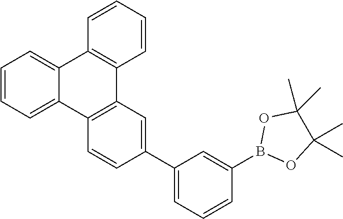 [SMILES: CC1(C)OB(c2cccc(-c3ccc4c5ccccc5c5ccccc5c4c3)c2)OC1(C)C] | 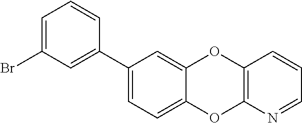 [SMILES: Brc1cccc(-c2ccc3c(c2)Oc2cccnc2O3)c1] | 564 |

TABLE 5-2

| Exem- | Exem- | | | m/z |
|---|---|---|---|---|
| plary | plary | | | |
| embod- | com- | | | |
| iment | pound | Raw material G1 | Raw material G2 | |
| 11 | B4 | 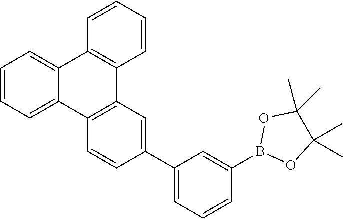 [SMILES: CC1(C)OB(c2cccc(-c3ccc4c5ccccc5c5ccccc5c4c3)c2)OC1(C)C] | 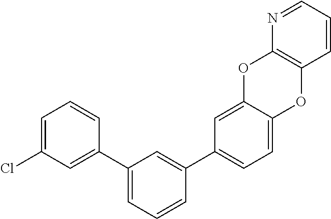 [SMILES: Clc1cccc(-c2cccc(-c3ccc4c(c3)Oc3ncccc3O4)c2)c1] | 640 |
| 12 | B5 | 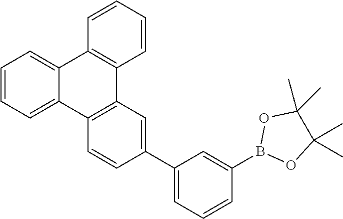 [SMILES: CC1(C)OB(c2cccc(-c3ccc4c5ccccc5c5ccccc5c4c3)c2)OC1(C)C] | 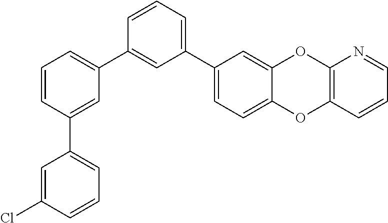 [SMILES: Clc1cccc(-c2cccc(-c3cccc(-c4ccc5c(c4)Oc4ncccc4O5)c3)c2)c1] | 716 |
| 13 | B8 | 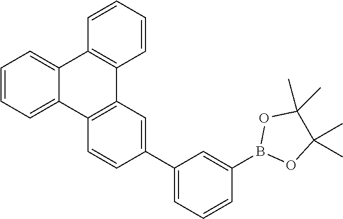 [SMILES: CC1(C)OB(c2cccc(-c3ccc4c5ccccc5c5ccccc5c4c3)c2)OC1(C)C] | 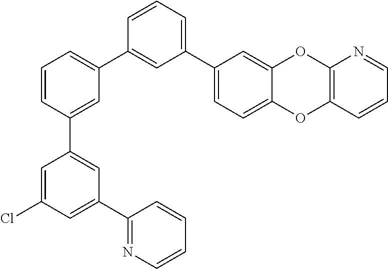 [SMILES: Clc1cc(-c2cccc(-c3cccc(-c4ccc5c(c4)Oc4ncccc4O5)c3)c2)cc(-c2ccccn2)c1] | 793 |
| 14 | B10 | 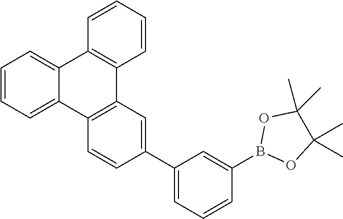 [SMILES: CC1(C)OB(c2cccc(-c3ccc4c5ccccc5c5ccccc5c4c3)c2)OC1(C)C] | 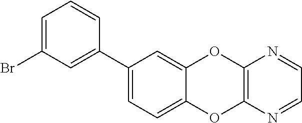 [SMILES: Brc1cccc(-c2ccc3c(c2)Oc2nccnc2O3)c1] | 565 |
| 15 | B11 | 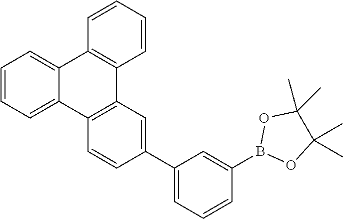 [SMILES: CC1(C)OB(c2cccc(-c3ccc4c5ccccc5c5ccccc5c4c3)c2)OC1(C)C] | 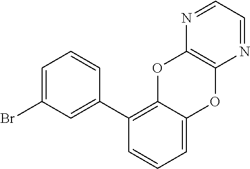 [SMILES: Brc1cccc(-c2cccc3c2Oc2nccnc2O3)c1] | 565 |
| 16 | B12 | 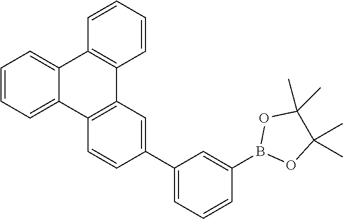 [SMILES: CC1(C)OB(c2cccc(-c3ccc4c5ccccc5c5ccccc5c4c3)c2)OC1(C)C] | 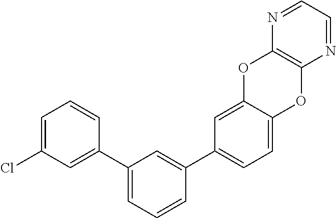 [SMILES: Clc1cccc(-c2cccc(-c3ccc4c(c3)Oc3nccnc3O4)c2)c1] | 641 |
| 17 | B14 | 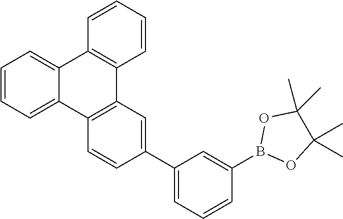 [SMILES: CC1(C)OB(c2cccc(-c3ccc4c5ccccc5c5ccccc5c4c3)c2)OC1(C)C] | 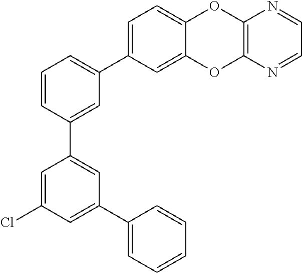 [SMILES: Clc1cc(-c2ccccc2)cc(-c2cccc(-c3ccc4c(c3)Oc3nccnc3O4)c2)c1] | 717 |
| 18 | B20 | 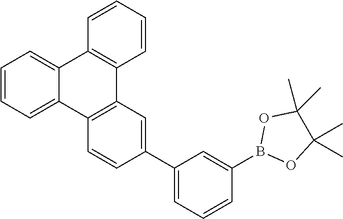 [SMILES: CC1(C)OB(c2cccc(-c3ccc4c5ccccc5c5ccccc5c4c3)c2)OC1(C)C] | 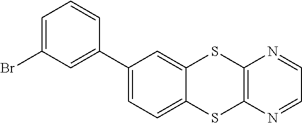 [SMILES: Brc1cccc(-c2ccc3c(c2)Sc2nccnc2S3)c1] | 597 |
| 19 | B25 | 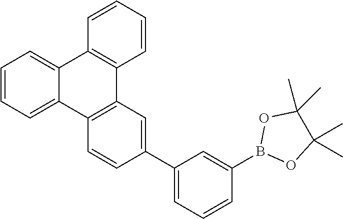 [SMILES: CC1(C)OB(c2cccc(-c3ccc4c5ccccc5c5ccccc5c4c3)c2)OC1(C)C] | 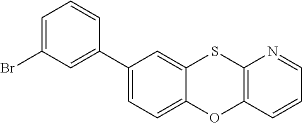 [SMILES: Brc1cccc(-c2ccc3c(c2)Sc2ncccc2O3)c1] | 580 |
| 20 | C4 | 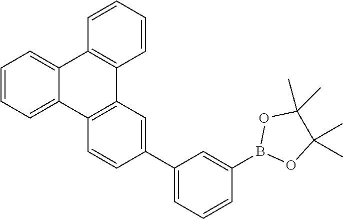 [SMILES: CC1(C)OB(c2cccc(-c3ccc4c5ccccc5c5ccccc5c4c3)c2)OC1(C)C] | 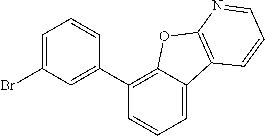 [SMILES: Brc1cccc(-c2cccc3c2oc2ncccc23)c1] | 564 |

TABLE 5-3

| Exem- | Exem- | | | |
|---|---|---|---|---|
| plary | plary | | | |
| embod- | com- | | | |
| iment | pound | Raw material G1 | Raw material G2 | m/z |
| 21 | D8 | 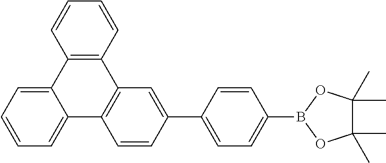 [SMILES: CC1(C)OB(c2ccc(-c3ccc4c5ccccc5c5ccccc5c4c3)cc2)OC1(C)C] | 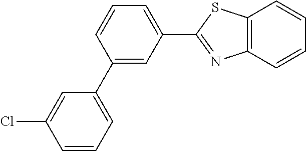 [SMILES: Clc1cccc(-c2cccc(-c3nc4ccccc4s3)c2)c1] | 590 |
| 22 | D17 | 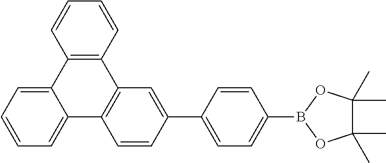 [SMILES: CC1(C)OB(c2ccc(-c3ccc4c5ccccc5c5ccccc5c4c3)cc2)OC1(C)C] | 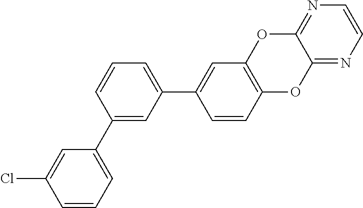 [SMILES: Clc1cccc(-c2cccc(-c3ccc4c(c3)Oc3nccnc3O4)c2)c1] | 641 |
| 23 | D21 | 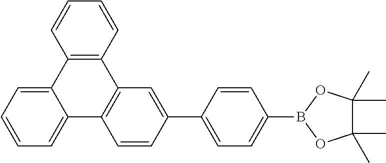 [SMILES: CC1(C)OB(c2ccc(-c3ccc4c5ccccc5c5ccccc5c4c3)cc2)OC1(C)C] | 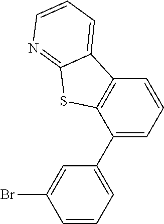 [SMILES: Brc1cccc(-c2cccc3c2sc2ncccc23)c1] | 564 |

| | | | |
|---|---|---|---|
| Comparative compound 1-a | 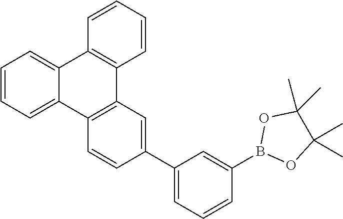 [SMILES: CC1(C)OB(c2cccc(-c3ccc4c5ccccc5c5ccccc5c4c3)c2)OC1(C)C] | 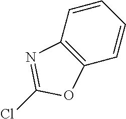 | 422 |
| Comparative compound 2-b | 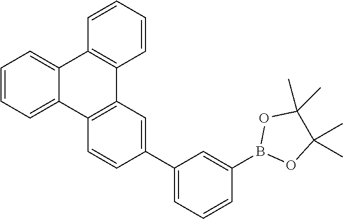 [SMILES: CC1(C)OB(c2cccc(-c3ccc4c5ccccc5c5ccccc5c4c3)c2)OC1(C)C] | 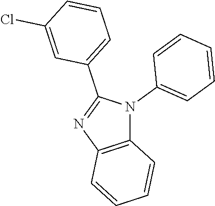 [SMILES: Clc1cccc(-c2nc3ccccc3n2-c2ccccc2)c1] | 573 |
| Comparative compound 3-a | 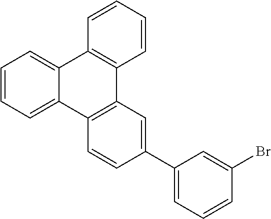 [SMILES: Brc1cccc(-c2ccc3c4ccccc4c4ccccc4c3c2)c1] | 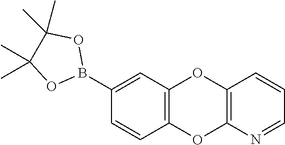 [SMILES: CC1(C)OB(c2ccc3c(c2)Oc2cccnc2O3)OC1(C)C] | 488 |
| Comparative compound 4-a | 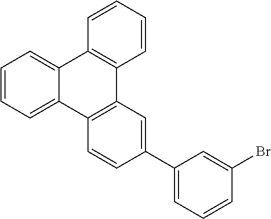 [SMILES: Brc1cccc(-c2ccc3c4ccccc4c4ccccc4c3c2)c1] | 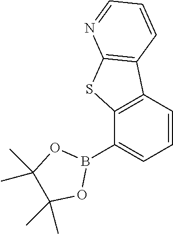 [SMILES: CC1(C)OB(c2cccc3c2sc2ncccc23)OC1(C)C] | 488 |
| Comparative compound 5-a | 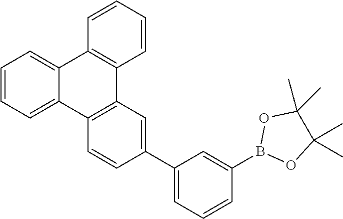 [SMILES: CC1(C)OB(c2cccc(-c3ccc4c5ccccc5c5ccccc5c4c3)c2)OC1(C)C] | 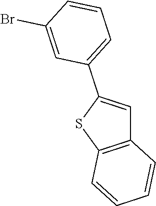 [SMILES: Brc1cccc(-c2cc3ccccc3s2)c1] | 513 |
| Comparative compound 6-a | 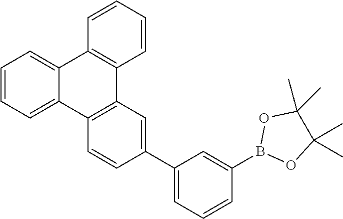 [SMILES: CC1(C)OB(c2cccc(-c3ccc4c5ccccc5c5ccccc5c4c3)c2)OC1(C)C] | 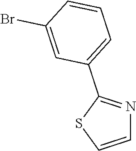 [SMILES: Brc1cccc(-c2nccs2)c1] | 464 |

### Exemplary Embodiment 24

An organic light-emitting element of a bottom emission type was produced. The organic light-emitting element included a positive electrode, a hole-injection layer, a hole-transport layer, an electron-blocking layer, a light-emitting layer, a hole-blocking layer, an electron-transport layer, an electron-injection layer, and a negative electrode sequentially formed on a substrate.

First, an ITO film was formed on a glass substrate and was subjected to desired patterning to form an ITO electrode (positive electrode). The ITO electrode had a thickness of 100 nm. The substrate on which the ITO electrode was formed was used as an ITO substrate in the following process. Vacuum deposition was then performed by resistance heating in a vacuum chamber at 1.33×10⁻⁴ Pa to continuously form an organic compound layer and an electrode layer shown in Table 6 on the ITO substrate. The counter electrode (a metal electrode layer, a negative electrode) had an electrode area of 3 mm².

TABLE 6

| | | | | Thickness |
|---|---|---|---|---|
| | Material | | | (nm) |
| Negative electrode | Al | | | 100 |
| electron-injection layer(EIL) | LiF | | | 1 |
| Electron-transport layer (ETL) | ET2 | | | 20 |
| Hole-blocking layer (HBL) | ET11 | | | 20 |
| Light-emitting layer (EML) | Host | A1 | Weight ratio | 20 |
| | Guest | GD10 | A1:AA2 = | |
| | | | 90:10 | |

| | | |
|---|---|---|
| Electron-blocking layer (EBL) | HT19 | 15 |
| Hole-transport layer (HTL) | HT3 | 30 |
| Hole-injection layer (HIL) | HT16 | 5 |

The characteristics of the element were measured and evaluated. The light-emitting element had a maximum external quantum efficiency (E.Q.E.) of 13%. A continuous operation test was performed at a current density of 100 mA/cm² to measure the time when the luminance decay rate reached 5%. The time when the luminance decay rate of Comparative Example 1 reached 5% was taken to be 1.0. The present exemplary embodiment had a luminance decay rate ratio of 1.2.

In the present exemplary embodiment, with respect to measuring apparatuses, more specifically, the current-voltage characteristics were measured with a microammeter 4140B manufactured by Hewlett-Packard Co., and the luminous brightness was measured with a BM7 manufactured by Topcon Corporation.

### Exemplary Embodiments 25 to 42 and Comparative Examples 1 to 6

Organic light-emitting elements were produced in the same manner as in Exemplary Embodiment 24 except that the compounds shown in Table 7 were used. Characteristics of the elements were measured and evaluated in the same manner as in Exemplary Embodiment 24. Tables 7 and 8 show the measurement results.

TABLE 7

| | | | | EML | | | | E.Q.E | Luminance decay |
|---|---|---|---|---|---|---|---|---|---|
| | HIL | HTL | EBL | Host | Guest | HBL | ETL | [%] | rate ratio |
| Exemplary | HT16 | HT3 | HT19 | A1 | AA2 | ET11 | ET2 | 14 | 1.3 |
| Embodiment 25 | | | | | | | | | |
| Exemplary | HT16 | HT2 | HT15 | A4 | GD15 | ET12 | ET2 | 14 | 1.2 |
| Embodiment 26 | | | | | | | | | |
| Exemplary | HT16 | HT3 | HT19 | A13 | GD10 | ET11 | ET2 | 13 | 1.2 |
| Embodiment 27 | | | | | | | | | |
| Exemplary | HT16 | HT3 | HT19 | A15 | BB25 | ET11 | ET2 | 14 | 1.3 |
| Embodiment 28 | | | | | | | | | |
| Exemplary | HT16 | HT3 | HT19 | A20 | GD15 | ET11 | ET15 | 14 | 1.3 |
| Embodiment 29 | | | | | | | | | |
| Exemplary | HT16 | HT3 | HT19 | B2 | GD10 | ET11 | ET2 | 14 | 1.3 |
| Embodiment 30 | | | | | | | | | |
| Exemplary | HT16 | HT3 | HT19 | B2 | DD31 | ET11 | ET2 | 15 | 1.4 |
| Embodiment 31 | | | | | | | | | |
| Exemplary | HT16 | HT2 | HT15 | B4 | GD15 | ET12 | ET15 | 14 | 1.4 |
| Embodiment 32 | | | | | | | | | |
| Exemplary | HT16 | HT3 | HT19 | B8 | GD10 | ET11 | ET15 | 14 | 1.3 |
| Embodiment 33 | | | | | | | | | |
| Exemplary | HT16 | HT3 | HT19 | B10 | GD10 | ET11 | ET2 | 14 | 1.3 |
| Embodiment 34 | | | | | | | | | |
| Exemplary | HT16 | HT3 | HT19 | B12 | DD31 | ET11 | ET2 | 15 | 1.4 |
| Embodiment 35 | | | | | | | | | |
| Exemplary | HT16 | HT3 | HT19 | B25 | GD10 | ET11 | ET2 | 14 | 1.3 |
| Embodiment 36 | | | | | | | | | |
| Exemplary | HT16 | HT3 | HT19 | C4 | GD10 | ET11 | ET2 | 14 | 1.3 |
| Embodiment 37 | | | | | | | | | |
| Exemplary | HT16 | HT2 | HT15 | C4 | DD31 | ET12 | ET15 | 15 | 1.4 |
| Embodiment 38 | | | | | | | | | |
| Exemplary | HT16 | HT3 | HT19 | D8 | GD10 | ET11 | ET2 | 12 | 1.2 |
| Embodiment 39 | | | | | | | | | |
| Exemplary | HT16 | HT3 | HT19 | D17 | GD15 | ET11 | ET2 | 13 | 1.2 |
| Embodiment 40 | | | | | | | | | |
| Exemplary | HT16 | HT3 | HT19 | B2 | GD10 | B10 | ET2 | 14 | 1.4 |
| Embodiment 41 | | | | | | | | | |
| Exemplary | HT16 | HT3 | HT19 | B10 | GD10 | B10 | ET2 | 14 | 1.4 |
| Embodiment 42 | | | | | | | | | |
| Comparative | HT16 | HT3 | HT19 | Comparative | GD10 | ET11 | ET2 | 12 | 1.0 |
| Example 1 | | | | compound 1-a | | | | | |
| Comparative | HT16 | HT3 | HT19 | Comparative | GD10 | ET11 | ET2 | 11 | 0.8 |
| Example 2 | | | | compound 2-b | | | | | |
| Comparative | HT16 | HT3 | HT19 | Comparative | GD10 | ET11 | ET2 | 12 | 1.0 |
| Example 3 | | | | compound 3-a | | | | | |
| Comparative | HT16 | HT3 | HT19 | Comparative | GD10 | ET11 | ET2 | 12 | 1.0 |
| Example 4 | | | | compound 4-a | | | | | |
| Comparative | HT16 | HT3 | HT19 | Comparative | GD10 | ET11 | ET2 | 12 | 1.1 |
| Example 5 | | | | compound 5-a | | | | | |
| Comparative | HT16 | HT3 | HT19 | Comparative | GD10 | ET11 | ET2 | 10 | 0.7 |
| Example 6 | | | | compound 6-a | | | | | |

Table 7 shows that the maximum external quantum efficiency (E.Q.E.) of Comparative Examples 1 to 6 ranged from 10% to 12%, and the light-emitting elements according to the present embodiments had higher light emission efficiency. Furthermore, the light-emitting elements according to the present embodiments had a longer life. This is because an organic compound according to the present embodiment has a high T₁, has HOMO-LUMO orbitals responsible for T₁ transition present on the unit A, and has a nitrogen-carbon-chalcogen atom skeleton (N═C—X) that promotes the interaction with an Ir complex. This is also due to its high thermal stability and bond dissociation energy. Furthermore, Exemplary Embodiments 25, 28, 31, 35, and 38 show that a light-emitting element with a particularly high efficiency and a long life could be produced by selecting a phosphorescent organometallic complex with a tricyclic or higher cyclic fused ring in a ligand suitable for combination with an organic compound according to the present embodiment. Thus, the organic compounds according to the present embodiments can be used to provide elements with high efficiency and durability.

### Exemplary Embodiment 43

An organic light-emitting element was produced in the same manner as in Exemplary Embodiment 24 except that the organic compound layer and the electrode layer shown in Table 8 were continuously formed.

TABLE 8

| | | | | Thickness |
|---|---|---|---|---|
| | Material | | | (nm) |
| Negative electrode | Al | | | 100 |
| electron-injection layer(EIL) | LiF | | | 1 |
| Electron-transport layer (ETL) | ET2 | | | 20 |
| Hole-blocking layer (HBL) | ET11 | | | 20 |
| Light-emitting layer (EML) | Host | A1 | Weight ratio | 20 |
| | Guest | AA22 | A1:AA22:EM30 = | |
| | Assist | EM30 | 60:10:30 | |

| | | |
|---|---|---|
| Electron-blocking layer (EBL) | HT19 | 15 |
| Hole-transport layer (HTL) | HT3 | 30 |
| Hole-injection layer (HIL) | HT16 | 5 |

The characteristics of the element were measured and evaluated. The light-emitting element had a green emission color and a maximum external quantum efficiency (E.Q.E.) of 18%.

### Exemplary Embodiments 44 to 56

Organic light-emitting elements were produced in the same manner as in Exemplary Embodiment 43 except that the compounds shown in Table 9 were used. Characteristics of the elements were measured and evaluated in the same manner as in Exemplary Embodiment 43. Table 9 shows the measurement results.

TABLE 9

| | | | | EML | | | | | E.Q.E |
|---|---|---|---|---|---|---|---|---|---|
| | HIL | HTL | EBL | Host | Guest | Assist | HBL | ETL | [%] |
| Exemplary | HT16 | HT3 | HT19 | A4 | BB24 | EM29 | ET26 | ET3 | 18 |
| Embodiment 44 | | | | | | | | | |
| Exemplary | HT16 | HT2 | HT15 | A13 | BB21 | EM35 | ET13 | ET2 | 18 |
| Embodiment 45 | | | | | | | | | |
| Exemplary | HT16 | HT2 | HT15 | A15 | CC17 | EM37 | ET13 | ET2 | 18 |
| Embodiment 46 | | | | | | | | | |
| Exemplary | HT16 | HT3 | HT19 | A20 | DD5 | EM13 | ET16 | ET15 | 18 |
| Embodiment 47 | | | | | | | | | |
| Exemplary | HT16 | HT3 | HT19 | B2 | DD31 | EM30 | ET16 | ET15 | 19 |
| Embodiment 48 | | | | | | | | | |
| Exemplary | HT16 | HT3 | HT19 | B4 | DD27 | EM28 | ET17 | ET15 | 19 |
| Embodiment 49 | | | | | | | | | |
| Exemplary | HT16 | HT3 | HT19 | B8 | EE1 | EM39 | ET13 | ET2 | 19 |
| Embodiment 50 | | | | | | | | | |
| Exemplary | HT16 | HT2 | HT15 | B10 | EE2 | GD10 | ET15 | ET3 | 19 |
| Embodiment 51 | | | | | | | | | |
| Exemplary | HT16 | HT3 | HT19 | B12 | FF2 | ET15 | ET15 | ET15 | 19 |
| Embodiment 52 | | | | | | | | | |
| Exemplary | HT16 | HT2 | HT15 | B25 | FF23 | ET16 | ET2 | ET2 | 19 |
| Embodiment 53 | | | | | | | | | |
| Exemplary | HT16 | HT3 | HT19 | C4 | FF1 | EM16 | ET26 | ET3 | 19 |
| Embodiment 54 | | | | | | | | | |
| Exemplary | HT16 | HT2 | HT15 | D8 | GG3 | EM16 | ET13 | ET2 | 17 |
| Embodiment 55 | | | | | | | | | |
| Exemplary | HT16 | HT2 | HT15 | D17 | HH25 | EM34 | ET13 | ET2 | 17 |
| Embodiment 56 | | | | | | | | | |

Exemplary Embodiments 43, 45, 46, 48 to 50, 52, 53, and 56 in Table 9 show that the light emission efficiency of the elements was improved by using an organic compound according to the present embodiment and further using a material with a carbazole skeleton, an azine ring, or a xanthone skeleton suitable for combination with the organic compound according to the present embodiment as an assist material.

An organic compound according to the present disclosure has high light emission efficiency and durability. A light-emitting layer containing an organic compound according to the present disclosure and an organometallic complex can be used to provide an organic light-emitting element with high light emission efficiency and durability.

While the present disclosure has been described with reference to exemplary embodiments, it is to be understood that the disclosure is not limited to the disclosed exemplary embodiments. The scope of the following claims is to be accorded the broadest interpretation so as to encompass all such modifications and equivalent structures and functions.

This application claims the benefit of Japanese Patent Application No. 2022-065793 filed Apr. 12, 2022, which is hereby incorporated by reference herein in its entirety.

What is claimed is:

1. An organic compound represented by the following formula [1]:
A-(B)ₘ [1]
wherein the organic compound satisfies (i) and (ii),
(i) T₁ of the unit A<T₁ of the unit B, and
(ii) highest occupied molecular orbital (HOMO)-lowest unoccupied molecular orbital (LUMO) orbitals responsible for T₁ transition are present in the unit A, and
the organic compound is represented by the following formula [2-1] or [2-2]: 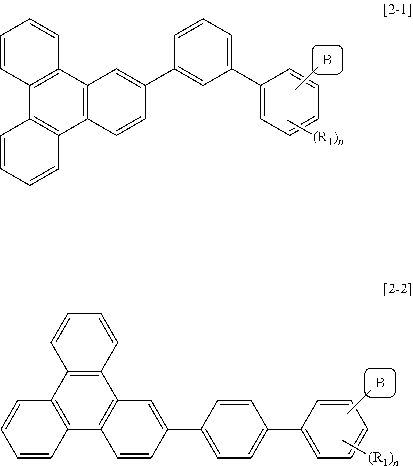
wherein R₁ is independently selected from a deuterium atom, substituted or unsubstituted alkyl groups, substituted or unsubstituted aryl groups, and substituted or unsubstituted heterocyclic groups, provided that R₁ is bonded via a carbon atom when R₁ denotes a substituted or unsubstituted heterocyclic group,
m denotes an integer of 1 or more and 5 or less, and n denotes an integer of 0 or more and 4 or less, and
the unit B is a fused ring with a partial structure represented by the following formula [3-1] or [3-2] or is a combination of the fused ring and a substituted or unsubstituted aromatic ring, and is bonded to the unit A via a carbon atom, 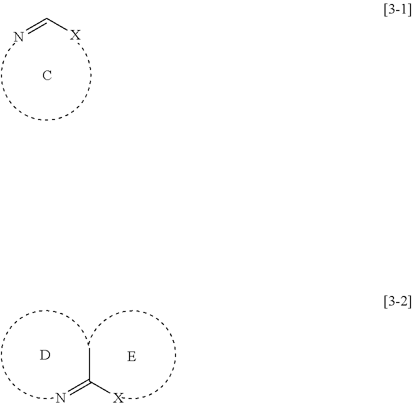
wherein X is selected from an oxygen atom, a sulfur atom, a selenium atom, and a tellurium atom, and
rings C to E independently denote a single ring and may have a deuterium atom or a substituted or unsubstituted alkyl group.

2. The organic compound according to claim 1, represented by formula [2-1].

3. The organic compound according to claim 1, wherein the fused ring is a bicyclic or tricyclic fused ring.

4. The organic compound according to claim 3, wherein the fused ring is selected from [301] to [340]:

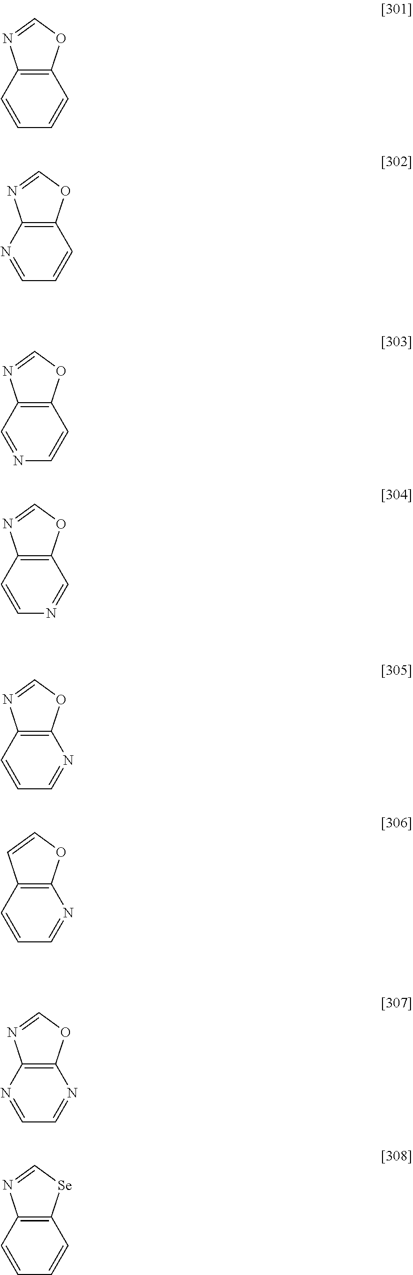

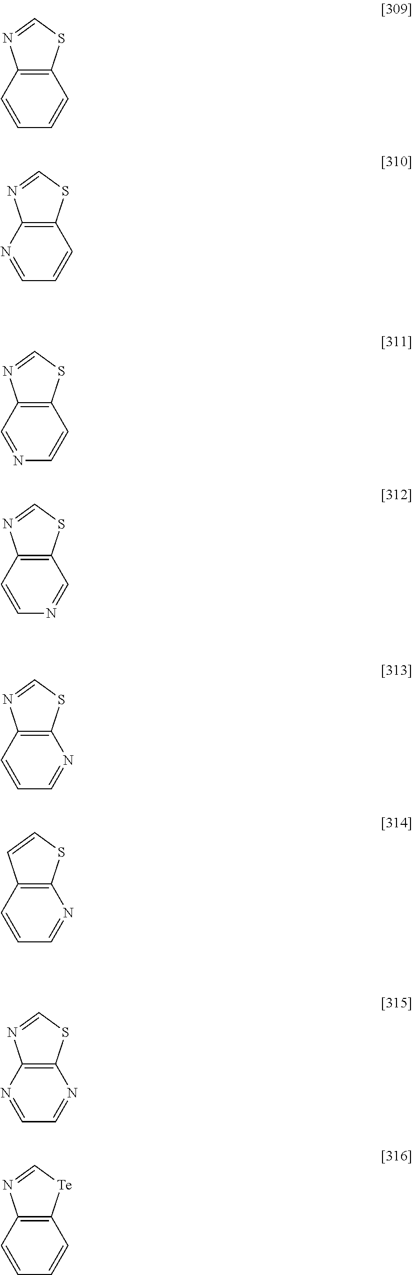

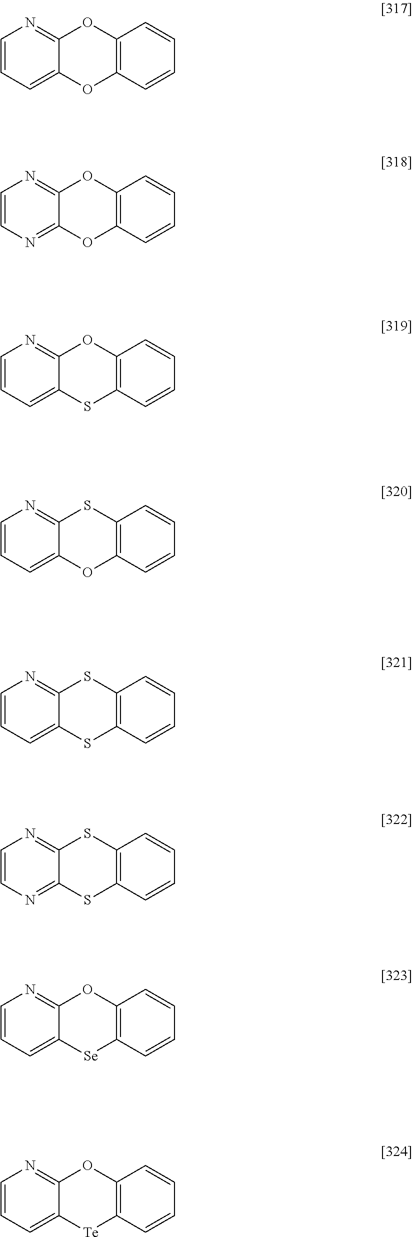

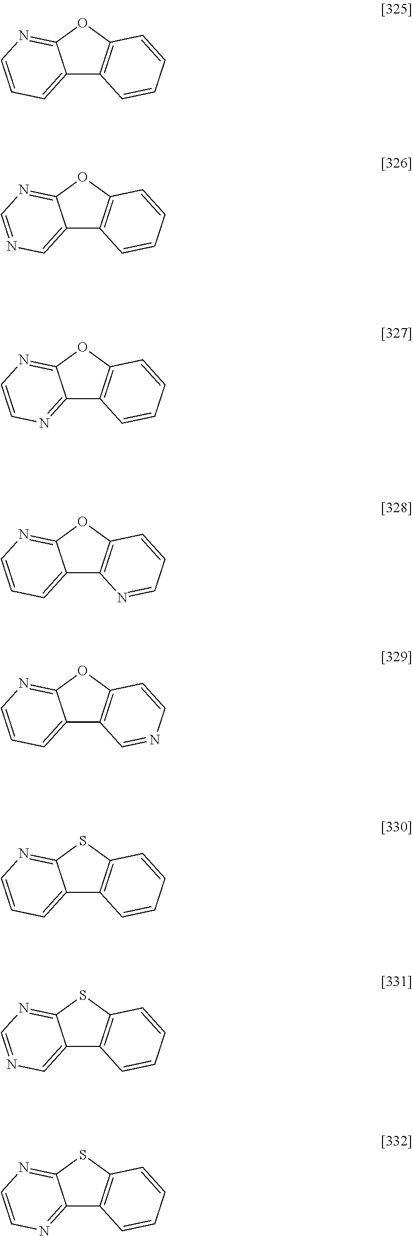

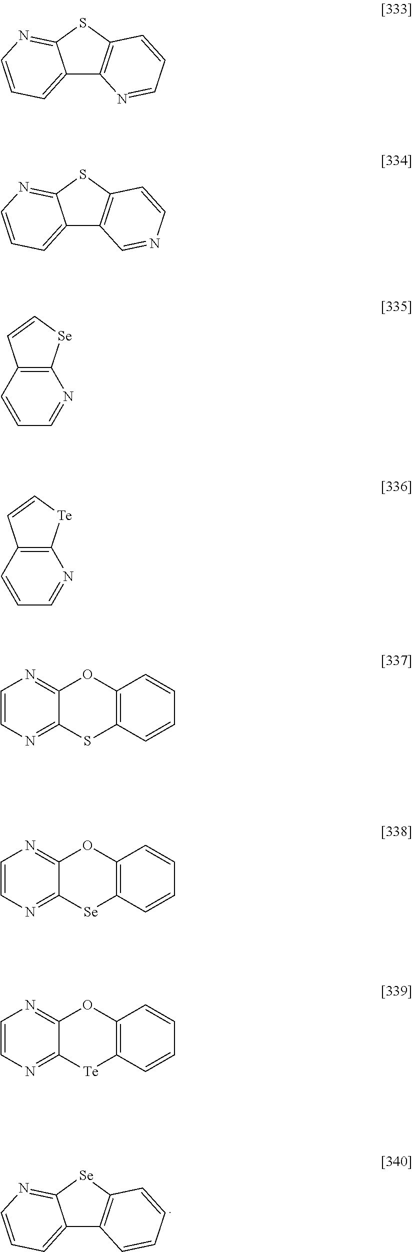

5. An organic light-emitting element comprising:
a pair of electrodes; and
a light-emitting layer between the pair of electrodes, wherein the light-emitting layer has at least a first organic compound and a phosphorescent organometallic complex, and
the first organic compound is the organic compound according to claim 1.

6. The organic light-emitting element according to claim 5, wherein the phosphorescent organometallic complex is an iridium complex.

7. The organic light-emitting element according to claim 6, wherein the phosphorescent organometallic complex has a tricyclic or higher cyclic fused ring.

8. The organic light-emitting element according to claim 7, wherein the tricyclic or higher cyclic fused ring is a ring selected from the group consisting of a phenanthrene ring, a triphenylene ring, a benzofluorene ring, a dibenzofuran ring, a dibenzothiophene ring, a benzonaphthofuran ring, a benzonaphthothiophene ring, a benzoisoquinoline ring, and a naphthoisoquinoline ring.

9. The organic light-emitting element according to claim 5, wherein the light-emitting layer further contains a third component.

10. The organic light-emitting element according to claim 9, wherein the third component has at least a carbazole skeleton.

11. The organic light-emitting element according to claim 9, wherein the third component has at least an azine ring in a skeleton thereof.

12. The organic light-emitting element according to claim 9, wherein the third component has at least xanthone in a skeleton thereof.

13. The organic light-emitting element according to claim 5, further comprising another light-emitting layer on the light-emitting layer, wherein the other light-emitting layer emits light of a different color from the light-emitting layer.

14. A display apparatus comprising a plurality of pixels, wherein at least one of the plurality of pixels includes the organic light-emitting element according to claim 5 and a transistor coupled to the organic light-emitting element.

15. An imaging apparatus comprising:
an optical unit including a plurality of lenses;
an imaging element configured to receive light passing through the optical unit; and
a display unit configured to display an image taken by the imaging element,
wherein the display unit includes the organic light-emitting element according to claim 5.

16. Electronic equipment comprising:
a display unit including the organic light-emitting element according to claim 5;
a housing in which the display unit is provided; and
a communication unit provided in the housing and configured to communicate with an outside.

17. Alighting apparatus comprising:
a light source including the organic light-emitting element according to claim 5; and
a light-diffusing unit or an optical filter configured to transmit light emitted by the light source.

18. A moving body comprising:
a lamp including the organic light-emitting element according to claim 5; and
a body to which the lamp is provided.

19. An exposure light source for an electrophotographic image-forming apparatus, comprising the organic light-emitting element according to claim 5.
